(19) United States
(12) Patent Application Publication
(10) Pub. No.: US 2026/0256780 A1
(43) Pub. Date: Sep. 3, 2026

(54) COMPOSITIONS FOR INDUCING RAS GTP HYDROLYSIS AND USES THEREOF

(71) Applicant: Revolution Medicines, Inc., Redwood City, CA (US)

(72) Inventors: John E. Knox, Emerald Hills, CA (US); Elena S. KOLTUN, Foster City, CA (US); Kyle SEAMON, San Mateo County, CA (US); Aidan TOMLINSON, San Francisco, CA (US); David E. WILDES, San Francisco, CA (US); James CREGG, Belmont, CA (US)

(21) Appl. No.: 19/339,640

(22) Filed: Sep. 25, 2025

Related U.S. Application Data
(63) Continuation of application No. PCT/US2024/022279, filed on Mar. 29, 2024.
(60) Provisional application No. 63/455,672, filed on Mar. 30, 2023.
(60) Provisional application No. 63/528,255, filed on Jul. 21, 2023.

(51) Int. Cl. A61K 31/504; A61K 31/497; A61K 31/519; A61K 31/5383; A61K 31/5386

(52) U.S. Cl. CPC A61K 31/504; A61K 31/497; A61K 31/519; A61K 31/5383; A61K 31/5386

(57) ABSTRACT

The present disclosure relates to therapies for treating cancer. In particular, the present disclosure relates to methods of treating cancer in a subject in need thereof, comprising administering to the subject a therapeutically effective amount of a RAS(ON) GTP hydrolysis-promoting compound optionally in combination with an additional therapeutic agent (e.g., a RAS(OFF) inhibitor, RTK inhibitor, SHP2 inhibitor, or SOS1 inhibitor), pharmaceutical compositions comprising a therapeutically effective amounts of the inhibitor(s), kits comprising the compositions and methods of use therefor.

### BACKGROUND

Cancer remains one of the most-deadly threats to human health. In the U.S., cancer affects nearly 1.3 million new patients each year, and is the second leading cause of death after heart disease, accounting for approximately 1 in 4 deaths.

RAS (KRAS, NRAS, HRAS) proteins regulate cell growth and other cellular functions by converting between a guanosine triphosphate (GTP)-bound “on” state (“RAS(ON)”) and a guanosine diphosphate (GDP)-bound “off” state (“RAS(OFF)”). The active state of RAS is bound to GTP, which is hydrolyzed to the GDP-bound inactive state. RAS proteins have a slow intrinsic hydrolysis rate (Westover et al., Mol Cancer Res (2015) 13 (9): 1325-1335), which is enhanced in the presence of RAS GTPase-activating proteins (GAPs). GDP-bound RAS can convert to the active state through a slow exchange of GDP for GTP, which is enhanced by guanine nucleotide exchange factors (GEFs). One way oncogenic mutations in RAS increase the amount of GTP-bound RAS protein is by decreasing the intrinsic hydrolysis rate and reducing sensitivity to GAP-mediated hydrolysis acceleration. Based on these observations, RAS mutants were historically thought to be ‘constitutively’ activated in cancer. The amount of active RAS can also be increased through activation of GEFs within the cell, either through mutations in upstream proteins (e.g., receptor tyrosine kinase mutations) or through non-mutational mechanisms (e.g., adaptive pathway reactivation). In either situation, the increase in GTP-bound RAS levels results in excessive cellular proliferation.

Covalent inhibitors of the “off” form of KRAS^{G12C} have demonstrated promising anti-tumor activity in cancer patients with oncogenic G12C mutations in KRAS. However, therapeutic inhibition of the RAS pathway, although often initially efficacious, can ultimately prove ineffective as it may, for example, lead to over-activation of RAS pathway signaling via a number of mechanisms including, e.g., reactivation of RAS pathway signaling via a number of mechanisms including, e.g., via relief of the negative feedback machineries that naturally operate in these pathways, or may lead to resistance to RAS(OFF) inhibitors. As a result, cancer cells that were initially sensitive to such inhibitors may become resistant. Most KRAS mutants are susceptible to inhibitors preferentially targeting their inactive state, suggesting that they retain the capacity to hydrolyze GTP in cancer cells. These findings prompt the search for pharmacologic interventions that enhance GTP hydrolysis by mutant KRAS.

Thus, a need exists in the art for compositions and methods which induce RAS GTP hydrolysis. Furthermore, there exists a need for compositions and methods which increase the sensitivity of a cancer cell to a RAS(OFF) inhibitor.

### SUMMARY

The present disclosure provides compositions and uses thereof for treating a RAS related disease or disorder (e.g., cancer) comprising a RAS(ON) GTP hydrolysis-promoting compound. For example, the disclosure provides combination therapies useful for treating cancer comprising the combination of a RAS(ON) GTP hydrolysis-promoting compound and an additional therapeutic agent (e.g., a RAS(OFF) inhibitor or a RAS degrader targeting the RAS(OFF) state (“RAS(OFF) degrader”). In any embodiment here employing a RAS(OFF) inhibitor, a RAS(OFF) degrader may be employed instead. In some embodiments, the combination comprises two or more therapeutic agents in addition to the RAS(ON) GTP hydrolysis-promoting compound (e.g., a RAS(OFF) inhibitor and a SHP2 inhibitor). In one aspect, the disclosure is based, at least in part, on the observation that contacting a cancer cell with a RAS(ON) GTP hydrolysis-promoting compound, and a RAS(OFF) inhibitor work synergistically in reducing cancer cell viability.

In another aspect, the disclosure provides a method of treating cancer in a subject in need thereof, the method comprising administering to the subject a therapeutically effective amount of a RAS(ON) GTP hydrolysis-promoting compound and a RAS(OFF) inhibitor, wherein the cancer does not comprise a RAS mutation at position 61. In some embodiments, the cancer comprises a RAS mutation (e.g., a RAS mutation is at position 12 or 13). In some embodiments, the cancer is pancreatic cancer, colorectal cancer, non-small cell lung cancer, gastric cancer, esophageal cancer, ovarian cancer, or uterine cancer. In some embodiments, the cancer is characterized by RAS amplification (RAS^{AMP}). In some embodiments, the RAS^{AMP} is wild-type RAS or mutant RAS.

In each of the above embodiments, binding of the RAS(ON) GTP hydrolysis-promoting compound to RAS GTP (RAS(ON)) alters the position of glutamine 61 of RAS(ON), relative to the position in the absence of the RAS(ON) GTP hydrolysis-promoting compound, towards the gamma phosphate of GTP bound to the RAS(ON) protein, thereby increasing the GTP hydrolysis rate relative to the hydrolysis rate of RAS(ON) in the absence of the RAS(ON) GTP hydrolysis-promoting compound.

In some embodiments, the RAS(ON) GTP-hydrolysis promoting compound is a compound of Formula Ia or Ib, or a pharmaceutically acceptable salt thereof. In exemplary embodiments, the RAS(ON) GTP-hydrolysis promoting compound is a compound of Table 1 or a pharmaceutically acceptable salt thereof.

In some embodiments, the RAS(OFF) inhibitor is a KRAS(OFF) inhibitor. In some embodiments, the KRAS(OFF) inhibitor is a KRAS^{G12C}(OFF) inhibitor. In some embodiments, the KRAS^{G12C}(OFF) inhibitor is selected from the group consisting of AMG510 (sotorasib), MRTX849 (adagrasib), MRTX1257, GDC-6036 (divarasib), JDQ443 (opnurasib), ERAS-3490, LY3537982 (olomorasib), BI 1823911, BPI-421286, JAB-3312, JAB-21000, JAB-21822 (glecirasib), D-1553, D3S-001, HBI-2438, HS-10370, MK-1084, YL-15293, BBO-8520 (ON/OFF inhibitor), FMC-376 (ON/OFF inhibitor), GEC255, and GFH925 (1B1351). In some embodiments, the KRAS(OFF) inhibitor is a KRAS^{G12D}(OFF) inhibitor. In some embodiments, the KRAS^{G12D}(OFF) inhibitor is selected from the group consisting of MRTX1133, MRTX282, JAB-22000, ERAS-4, ERAS-5024, HRS-4642, BI-2852, ASP3082, TH-Z827, TH-7835, QTX-3046, GFH375 (VS-7375), INCB161734 and KD-8.

In some embodiments, the KRAS(OFF) inhibitor is a KRAS^{G12V}(OFF) inhibitor. In specific embodiments, the KRAS^{G12V}(OFF) inhibitor is JAB-23000.

In some embodiments, the KRAS(OFF) inhibitor is a pan-RAS(OFF) inhibitor. In specific embodiments, the pan-RAS(OFF) inhibitor is JAB-23400, JAB-23425, BI-2493, BI-2865, QTX-3034 (G12D preferring), QTX3544 (G12V preferring), ZG2001, BBO-a, BBO-B, or pan KRas-IN-1.

In some embodiments, the RAS(ON) GTP hydrolysis-promoting compound and the RAS(OFF) inhibitor are administered on the same day. In some embodiments, the RAS(ON) GTP hydrolysis-promoting compound and the RAS(OFF) inhibitor are administered concurrently or sequentially. In some embodiments, the RAS(ON) GTP hydrolysis-promoting compound and the RAS(OFF) inhibitor are administered on different days.

In each of the preceding embodiments, the method may further comprise administering an additional anticancer therapy. In some embodiments, the additional anticancer therapy is an EGFR inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, an immune checkpoint inhibitor, a CDK4/6 inhibitor, a HER2 inhibitor, or a combination thereof. In some embodiments, the immune checkpoint inhibitor is a PD-L1 inhibitor or a PD-1 inhibitor. In some embodiments, the additional therapy is a SHP2 inhibitor or a SOS1 inhibitor. In each of the preceding embodiments, the SOS1 inhibitor is RMC-5845, RMC-4948, RMC-0331, BI-1701963, BI-3406, SDR5, MRTX0902, BAY-293, or any combination thereof. In each of the preceding embodiments, the SHP2 inhibitor is SHP099, TNO155, RMC-4550, RMC-4630, JAB-3068, JAB-3312, RLY-1971, ERAS-601, SH3809, PF-07284892, BBP-398, or any combination thereof.

In some embodiments, the subject's cancer progresses on the RAS(OFF) inhibitor (e.g., when the subject is administered the RAS(OFF) inhibitor in the absence of a RAS(ON) GTP hydrolysis-promoting compound). In some embodiments, the subject has been treated with a RAS(OFF) inhibitor (e.g., the subject has been previously treated with a RAS(OFF) inhibitor, e.g., prior to administration of the RAS(ON) GTP hydrolysis-promoting compound). In some embodiments, the subject has acquired resistance to a RAS(OFF) inhibitor (e.g., has acquired a mutation that confers resistance to a RAS(OFF) inhibitor, e.g., prior to administration of the RAS(ON) GTP hydrolysis-promoting compound).

In another aspect, the present disclosure provides methods of treating a RAS protein-related disorder in a subject in need thereof, the methods generally comprise administering to the subject a therapeutically effective amount of a RAS(ON) GTP hydrolysis-promoting compound.

In yet another aspect, the present disclosure provides method of treating a RASopathy in a subject in need thereof, the methods generally comprise administering to the subject a therapeutically effective amount of a RAS(ON) GTP hydrolysis-promoting compound. In some embodiments, the RASopathy is cardiofaciocutaneous syndrome, Costello syndrome, Legius syndrome, neurofibromatosis type 1, Noonan syndrome, or capillary malformation-arteriovenous malformation syndrome. In some embodiments, binding of the RAS(ON) GTP hydrolysis-promoting compound to RAS(ON) alters the position of glutamine 61 of RAS(ON), relative to the position in the absence of the GTP hydrolysis-promoting compound, towards the gamma phosphate of GTP bound to the RAS(ON) protein, thereby increasing the GTP hydrolysis rate relative to the hydrolysis rate of RAS(ON) in the absence of the RAS(ON) GTP hydrolysis-promoting compound. In some embodiments, the methods may further comprise administering an additional RASopathy therapy. In some embodiments, the additional RASopathy therapy is an EGFR inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a CDK4/6 inhibitor, a HER2 inhibitor, or a combination thereof.

In still yet another aspect, the present disclosure provides methods of inhibiting RAS activity in a cell, the methods generally comprise contacting the cell in which inhibition of RAS activity is desired with an effective amount of a RAS(ON) GTP hydrolysis-promoting compound. In some embodiments, the RAS(ON) GTP hydrolysis-promoting compound is provided in combination with a RAS(OFF) inhibitor, wherein the RAS(ON) GTP hydrolysis-promoting compound synergistically increases the sensitivity of the cell to the RAS(OFF) inhibitor.

In another aspect, the present disclosure provides methods of increasing sensitivity of a cell to a RAS(OFF) inhibitor, the methods generally comprise contacting the cell in which increasing the sensitivity to the RAS(OFF) inhibitor is desired with an effective amount of a RAS(ON) GTP hydrolysis-promoting compound, wherein the RAS(ON) GTP hydrolysis-promoting compound synergistically increases the sensitivity of the cell to the RAS(OFF) inhibitor.

In yet another aspect, the present disclosure provides methods of increasing the cross-linking rate of a KRAS^{G12C}(OFF) inhibitor to the cysteine residue at position 12 of KRAS^{G12C} in a cell comprising contacting the cell with an effective amount of a RAS(ON) GTP hydrolysis-promoting compound.

In another aspect, the present disclosure provides pharmaceutical compositions, comprising a therapeutically effective amount of a RAS(ON) GTP hydrolysis-promoting compound. In some embodiments, the pharmaceutical composition comprises a RAS(ON) GTP hydrolysis-promoting compound and a RAS(OFF) inhibitor.

In another aspect, the present disclosure provides kits comprising, a) a RAS(ON) GTP hydrolysis-promoting compound and b) a RAS(OFF) inhibitor. In some embodiments, the kits further comprise an insert with instructions for administration of the pharmaceutical composition(s).

It is specifically contemplated that any limitation discussed with respect to one embodiment of the disclosure may apply to any other embodiment of the disclosure. Furthermore, any compound or composition of the disclosure may be used in any method of the disclosure, and any method of the disclosure may be used to produce or to utilize any compound or composition of the disclosure.

### BRIEF DESCRIPTION OF THE FIGURES

FIG. 1 graphically depicts the characterization of the RAS GTP hydrolysis activation by a representative moderate RAS(ON) GTP hydrolysis-promoting compound (Compound E) in various RAS mutants. All RAS mutants show increased hydrolysis in the presence of the RAS(ON) GTP-hydrolysis-promoting compound, except those with mutations of the Q61 residue required for catalytical hydrolysis activity.

FIG. 2 is a graph showing the characterization of RAS GTP hydrolysis activation by various compounds. Compound F represents the class that does not activate GTP hydrolysis by RAS, while the others show varying degrees of RAS GTP hydrolysis activation.

FIG. 3A shows a best fit of the phospho-(Thr202/Tyr204; Thr185/Tyr187)-ERK1/2 concentration-response curves of Compound C in the presence or absence of 1 μM RMC-4550 in the KRASG12D-mutant cell line, AsPC-1.

FIG. 3B shows a best fit of the phospho-(Thr202/Tyr204; Thr185/Tyr187)-ERK1/2 concentration-response curves of Compound F in the presence or absence of 1 μM RMC-4550 in the KRASG12D-mutant cell line, AsPC-1.

FIG. 3C shows a best fit of cellular viability concentration-response curves of Compound C in the presence or absence of 1 μM RMC-4550 in the KRASG12D-mutant cell line, AsPC-1.

FIG. 3D shows a best fit of cellular viability concentration-response curves of Compound F in the presence or absence of 1 μM RMC-4550 in the KRASG12D-mutant cell line, AsPC-1.

FIG. 4A shows a best fit of the cellular viability concentration-response curves of Compound A and MRTX1133 in the KRASG12D-mutant cell line, AsPC-1.

FIG. 4B shows a HSA synergy model of Compound A and MRTX1133 in the KRAS^{G12D}-mutant cell line, AsPC-1.

FIG. 4C is a bar graph showing representative points of synergistic drug combination between Compound A and MRTX1133 in the KRAS^{G12D}-mutant cell line, AsPC-1.

FIG. 4D shows a best fit of the cellular viability concentration-response curves of Compound A and MRTX-282 in the KRAS^{G12D}-mutant cell line, AsPC-1.

FIG. 4E shows a HSA synergy model of Compound A and MRTX-282 in the KRAS^{G12D}-mutant cell line, AsPC-1.

FIG. 4F is a bar graph showing representative points of synergistic drug combination between Compound A and MRTX-282 in the KRAS^{G12D}-mutant cell line, AsPC-1.

FIG. 4G shows a best fit of the concentration-response curves of Compound D and MRTX1133 in the KRAS^{G12D}-mutant cell line, AsPC-1.

FIG. 4H shows a HSA synergy model of Compound D and MRTX1133 in the KRAS^{G12D}-mutant cell line, AsPC-1.

FIG. 4I is a bar graph showing representative points of synergistic drug combination between Compound D and MRTX1133 in the KRAS^{G12D}-mutant cell line, AsPC-1.

FIG. 5A shows a best fit of the cellular viability concentration-response curves of Compound A and MRTX1133 in the KRASG12D-mutant cell line, HPAC.

FIG. 5B shows a HSA synergy model of Compound A and MRTX1133 in the KRAS^{G12D}-mutant cell line, HPAC.

FIG. 5C is a bar graph showing representative points of synergistic drug combination between Compound A and MRTX1133 in the KRAS^{G12D}-mutant cell line, HPAC.

FIG. 5D shows a best fit of the cellular viability concentration-response curves of Compound A and MRTX-282 in the KRAS^{G12D}-mutant cell line, HPAC.

FIG. 5E shows a HSA synergy model of Compound A and MRTX-282 in the KRAS^{G12D}-mutant cell line, HPAC.

FIG. 5F is a bar graph showing representative points of synergistic drug combination between Compound A and MRTX-282 in the KRAS^{G12D}-mutant cell line, HPAC.

FIG. 6A shows a best fit of the cellular viability concentration-response curves of Compound A and MRTX1133 in the KRASG12D-mutant cell line, Gp2D.

FIG. 6B shows a HSA synergy model of Compound A and MRTX1133 in the KRAS^{G12D}-mutant cell line, Gp2D.

FIG. 6C is a bar graph showing representative points of synergistic drug combination between Compound A and MRTX1133 in the KRAS^{G12D}-mutant cell line, Gp2D.

FIG. 6D shows a best fit of the cellular viability concentration-response curves of Compound A and MRTX-282 in the KRAS^{G12D}-mutant cell line, Gp2D.

FIG. 6E shows a HSA synergy model of Compound A and MRTX-282 in the KRAS^{G12D}-mutant cell line, Gp2D.

FIG. 6F is a bar graph showing representative points of synergistic drug combination between Compound A and MRTX-282 in the KRAS^{G12D}-mutant cell line, Gp2D.

FIG. 7A shows a best fit of the cellular viability concentration response curves of Compound D and MRTX-282 in the RAS wild-type cell line, HaCat.

FIG. 7B shows a HSA synergy model of compound D and MRTX-282 in the RAS wild-type cell line, HaCat.

FIG. 7C is a bar graph showing representative points of the non-synergistic drug combination of Compound D and MRTX-282 in the RAS wild-type cell line, HaCat.

FIG. 7D shows a best fit of the cellular viability concentration response curves of Compound D and MRTX-282 in the RAS wild-type cell line, HaCat.

FIG. 7E shows a HSA synergy model of compound D and MRTX-282 in the RAS wild-type cell line, HaCat.

FIG. 7F is a bar graph showing representative points of the non-synergistic drug combination of Compound D and MRTX-282 in the RAS wild-type cell line, HaCat.

FIG. 8A shows a best fit of the cellular viability concentration-response curves of Compound A and AMG510 in the KRAS^{G12C}-mutant cell line, MiaPaCa2.

FIG. 8B shows a HSA synergy model of Compound A and AMG510 in the KRAS^{G12C}-mutant cell line, MiaPaCa2.

FIG. 8C is a bar graph showing representative points of synergistic drug combination between Compound A and AMG510 in the KRAS^{G12C}-mutant cell line, MiaPaCa2.

FIG. 8D shows a best fit of the cellular viability concentration-response curves of Compound A and MRTX849 in the KRAS^{G12C}-mutant cell line, MiaPaCa2.

FIG. 8E shows a HSA synergy model of Compound A and MRTX849 in the KRAS^{G12C}-mutant cell line, MiaPaCa2.

FIG. 8F is a bar graph showing representative points of synergistic drug combination between Compound A and MRTX849 in the KRAS^{G12C}-mutant cell line, MiaPaCa2.

FIG. 9A is an immunoblot of MiaPaCa2 cells showing total RAS separated into wild-type HRAS and NRAS, free KRAS^{G12C}, and KRAS^{G12C} covalently modified (cross-linked) by sotorasib.

FIG. 9B is a bar graph depicting the rate of covalent modification of KRAS^{G12C} by sotorasib in MiaPaCa2 cells alone or in combination with Compound A.

FIG. 10A shows a best fit of an intracellular RAS-RAF complex reporter assay concentration-response curve of Compound E across different KRAS mutant proteins.

FIG. 10B shows a best fit of an intracellular RAS-RAF complex reporter assay concentration-response curve of pan KRAS-IN-1 across different KRAS mutant proteins.

FIG. 10C shows a best fit of an intracellular KRAS^{G12V}-RAF complex reporter assay concentration-response curve of pan KRAS-IN-1 in the presence or absence of 1 μM Compound D.

FIG. 11A shows a best fit of the cellular viability concentration-response curves of Compound D and pan KRAS-IN-1 in the KRAS^{G12D}-mutant cell line, AsPC-1.

FIG. 11B shows a HSA synergy model of Compound D and pan KRAS-IN-1 in the KRAS^{G12D}-mutant cell line, AsPC-1.

FIG. 11C is a bar graph showing representative points of synergistic drug combination between Compound D and pan KRAS-IN-1 in the KRAS^{G12D}-mutant cell line, AsPC-1.

FIG. 11D shows a best fit of the cellular viability concentration-response curves of Compound C and pan KRAS-IN-1 in the KRAS^{G12D}-mutant cell line, AsPC-1.

FIG. 11E shows a HSA synergy model of Compound C and pan KRAS-IN-1 in the KRAS^{G12D}-mutant cell line, AsPC-1.

FIG. 11F is a bar graph showing representative points of synergistic drug combination between Compound C and pan KRAS-IN-1 in the KRAS^{G12D}-mutant cell line, AsPC-1.

FIG. 12A shows a best fit of the cellular viability concentration-response curves of Compound D and pan KRAS-IN-1 in the KRAS^{G12D}-mutant cell line, Capan-2.

FIG. 12B shows a HSA synergy model of Compound D and pan KRAS-IN-1 in the KRAS^{G12D}-mutant cell line, Capan-2.

FIG. 12C is a bar graph showing representative points of synergistic drug combination between Compound D and pan KRAS-IN-1 in the KRAS^{G12D}-mutant cell line, Capan-2.

FIG. 12D shows a best fit of the cellular viability concentration-response curves of Compound C and pan KRAS-IN-1 in the KRAS^{G12D}-mutant cell line, Capan-2.

FIG. 12E shows a HSA synergy model of Compound C and pan KRAS-IN-1 in the KRAS^{G12D}-mutant cell line, Capan-2.

FIG. 12F is a bar graph showing representative points of synergistic drug combination between Compound C and pan KRAS-IN-1 in the KRAS^{G12D}-mutant cell line, Capan-2.

FIG. 13A shows a best fit of the cellular viability concentration-response curves of Compound D and pan KRAS-IN-1 in the KRAS^{G12D}-mutant cell line, NCI-H358.

FIG. 13B shows a HSA synergy model of Compound D and pan KRAS-IN-1 in the KRAS^{G12D}-mutant cell line, NCI-H358.

FIG. 13C is a bar graph showing representative points of synergistic drug combination between Compound D and pan KRAS-IN-1 in the KRAS^{G12D}-mutant cell line, NCI-H358.

FIG. 13D shows a best fit of the cellular viability concentration-response curves of Compound C and pan KRAS-IN-1 in the KRAS^{G12D}-mutant cell line, NCI-H358.

FIG. 13E shows a HSA synergy model of Compound C and pan KRAS-IN-1 in the KRAS^{G12D}-mutant cell line, NCI-H358.

FIG. 13F is a bar graph showing representative points of synergistic drug combination between Compound C and pan KRAS-IN-1 in the KRAS^{G12D}-mutant cell line, NCI-H358.

FIG. 14A shows a best fit of the cellular viability concentration-response curves of Compound D and pan KRAS-IN-1 in the KRAS^{G12D}-mutant cell line, PSN-1.

FIG. 14B shows a HSA synergy model of Compound D and pan KRAS-IN-1 in the KRAS^{G12D}-mutant cell line, PSN-1.

FIG. 14C is a bar graph showing representative points of synergistic drug combination between Compound D and pan KRAS-IN-1 in the KRAS^{G12D}-mutant cell line, PSN-1.

FIG. 14D shows a best fit of the cellular viability concentration-response curves of Compound C and pan KRAS-IN-1 in the KRAS^{G12D}-mutant cell line, PSN-1.

FIG. 14E shows a HSA synergy model of Compound C and pan KRAS-IN-1 in the KRAS^{G12D}-mutant cell line, PSN-1.

FIG. 14F is a bar graph showing representative points of synergistic drug combination between Compound C and pan KRAS-IN-1 in the KRAS^{G12D}-mutant cell line, PSN-1.

FIG. 15 shows KRAS residue Q61 orientation differs between an exemplary non-RAS(ON) GTP hydrolysis-promoting compound containing a phenol at the A position of a Formula Ia- or Formula Ib-type compound and an exemplary RAS(ON) GTP hydrolysis-promoting compound containing a thiazole at the comparable position.

FIG. 16 shows a schematic depicting the mechanistic rationale for synergistic combination benefit between RAS(ON) GTP-hydrolysis-promoting compounds that accelerate GTP hydrolysis and RAS nucleotide exchange inhibitor.

FIG. 17 shows a schematic depicting the mechanistic rationale for synergistic combination benefit between RAS(ON) GTP-hydrolysis-promoting compounds that accelerate GTP hydrolysis and RAS inhibitors that bind with higher affinity to the RAS(OFF) [GDP-bound]state. Here a RAS(ON) GTP-hydrolysis-promoting compound is shown complexed with cyclophilin A (CypA), which then binds to RAS(ON) to form a tri-complex that then catalyzes the hydrolysis of GTP, converting RAS(ON) to RAS(OFF). The RAS(OFF) generated by this reaction is then bound by the RAS(OFF) inhibitor.

### DETAILED DESCRIPTION

The present disclosure relates generally to compositions and methods for modulating RAS activity for the treatment of cancer. In particular, the present disclosure provides therapies for cancers harboring a RAS mutation. In each embodiment, the cancer does not comprise a mutation at residue Q61. The present disclosure provides methods of treating cancer in a subject in need thereof, comprising administering to the subject a therapeutically effective amount of a RAS(ON) GTP hydrolysis-promoting compound, or a pharmaceutically acceptable salt or a pharmaceutical composition thereof, optionally in combination with a RAS(OFF) inhibitor, or a pharmaceutically acceptable salt or a pharmaceutical composition thereof. In any embodiment here employing a RAS(OFF) inhibitor, a RAS(OFF) degrader may be employed instead. The present disclosure also provides methods comprising a RAS(ON) GTP hydrolysis-promoting compound and an additional therapeutic agent (e.g., a SOS1 inhibitor, SHP2 inhibitor, an RTK inhibitor and/or additional RAS inhibitor). The present disclosure also provides pharmaceutical compositions comprising therapeutically effective amounts of the inhibitors, kits comprising the compositions and methods of use therefor.

Oncogenic RAS mutations that increase the proportion of RAS protein in the GTP-bound state limit the amount of GDP-bound RAS for a RAS(OFF) inhibitor to bind. Without wishing to be bound by theory, the inventors postulate that compounds as disclosed herein increase the rate of GTP hydrolysis by oncogenic RAS and/or wild-type RAS, thereby enhancing the potency of RAS(OFF) inhibitors by increasing the levels of GDP-bound RAS. The RAS(ON) GTP hydrolysis-promoting compounds disclosed herein show greater selectivity for RAS^{G12X} relative to RAS^{WT} due to the inherent GAP-deficiency of mutant RAS isoforms. Thus, RAS(ON) GTP hydrolysis-promoting compounds disclosed herein are useful in the context of RAS^{AMP} (e.g., mutant RAS^{AMP}) due to the catalytic rather than stoichiometric mechanism of target inhibition. Moreover, the unique profile of the RAS(ON) GTP hydrolysis-promoting compounds disclosed herein lends itself to improved tolerability including in the context of combination therapies, particularly in-pathway combinations.

### General Methods

The practice of the present disclosure will employ, unless otherwise indicated, conventional techniques of cell culturing, molecular biology (including recombinant techniques), microbiology, cell biology, biochemistry, and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

### Definitions

In this application, unless otherwise clear from context, (i) the term “a” means “one or more”; (ii) the term “or” is used to mean “and/or” unless explicitly indicated to refer to alternatives only or the alternative are mutually exclusive, although the disclosure supports a definition that refers to only alternatives and “and/or”; (iii) the terms “comprising” and “including” are understood to encompass itemized components or steps whether presented by themselves or together with one or more additional components or steps; and (iv) where ranges are provided, endpoints are included.

As used herein, the term “about” is used to indicate that a value includes the standard deviation of error for the device or method being employed to determine the value. In certain embodiments, the term “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used herein, the term “adjacent” in the context of describing adjacent atoms refers to bivalent atoms that are directly connected by a covalent bond.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of’ is meant including, and limited to, whatever follows the phrase “consisting of.” Thus, the phrase “consisting of’ indicates that the listed elements are required or mandatory, and that no other elements may be present. By “consisting essentially of’ is meant including any elements listed after the phrase and limited to other elements that do not interfere with or contribute to the activity or action specified in the disclosure for the listed elements. Thus, the phrase “consisting essentially of’ indicates that the listed elements are required or mandatory, but that other elements are optional and may or may not be present depending upon whether or not they materially affect the activity or action of the listed elements.

A “compound of the present disclosure” and similar terms as used herein, whether explicitly noted or not, refers to RAS(ON) GTP hydrolysis-promoting compounds described herein, including compounds of Formula Ia and Formula Ib and subformula thereof, and compounds of Table 1 and Table 2, as well as salts (e.g., pharmaceutically acceptable salts), solvates, hydrates, stereoisomers (including atropisomers), and tautomers thereof.

Those skilled in the art will appreciate that certain compounds described herein can exist in one or more different isomeric (e.g., stereoisomers, geometric isomers, atropisomers, tautomers) or isotopic (e.g., in which one or more atoms has been substituted with a different isotope of the atom, such as hydrogen substituted for deuterium) forms. Unless otherwise indicated or clear from context, a depicted structure can be understood to represent any such isomeric or isotopic form, individually or in combination.

Compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

In some embodiments, one or more compounds depicted herein may exist in different tautomeric forms. As will be clear from context, unless explicitly excluded, references to such compounds encompass all such tautomeric forms. In some embodiments, tautomeric forms result from the swapping of a single bond with an adjacent double bond and the concomitant migration of a proton. In certain embodiments, a tautomeric form may be a prototropic tautomer, which is an isomeric protonation states having the same empirical formula and total charge as a reference form. Examples of moieties with prototropic tautomeric forms are ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H—, 2H- and 4H-1,2,4-triazole, 1H- and 2H-isoindole, and 1H- and 2H-pyrazole. In some embodiments, tautomeric forms can be in equilibrium or sterically locked into one form by appropriate substitution. In certain embodiments, tautomeric forms result from acetal interconversion.

Unless otherwise stated, structures depicted herein are also meant to include compounds that differ only in the presence of one or more isotopically enriched atoms. Exemplary isotopes that can be incorporated into compounds of the present disclosure include isotopes of hydrogen, carbon, nitrogen, oxygen, phosphorus, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I and ¹²⁵I. Isotopically labeled compounds (e.g., those labeled with ³H and ¹⁴C) can be useful in compound or substrate tissue distribution assays. Tritiated (i.e., ³H) and carbon-14 (i.e., ¹⁴C) isotopes can be useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements). In some embodiments, one or more hydrogen atoms are replaced by ²H or ³H, or one or more carbon atoms are replaced by ¹³C- or ¹⁴C-enriched carbon. Positron emitting isotopes such as ¹⁵O, ¹³N, ¹¹C, and ¹⁸F are useful for positron emission tomography (PET) studies to examine substrate receptor occupancy. Preparations of isotopically labelled compounds are known to those of skill in the art. For example, isotopically labeled compounds can generally be prepared by following procedures analogous to those disclosed for compounds of the present disclosure described herein, by substituting an isotopically labeled reagent for a non-isotopically labeled reagent.

Non-limiting examples of moieties that may contain one or more deuterium substitutions in compounds of the present disclosure, where any position “R” may be deuterium (D), include

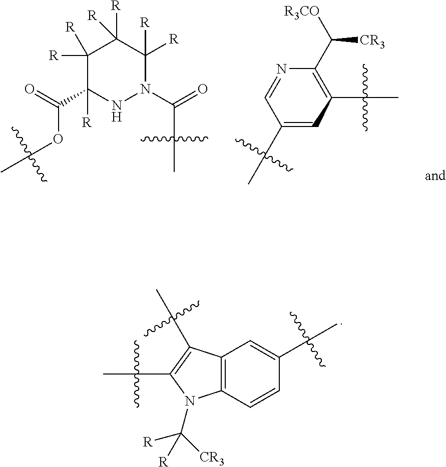

Additional examples include moieties such as

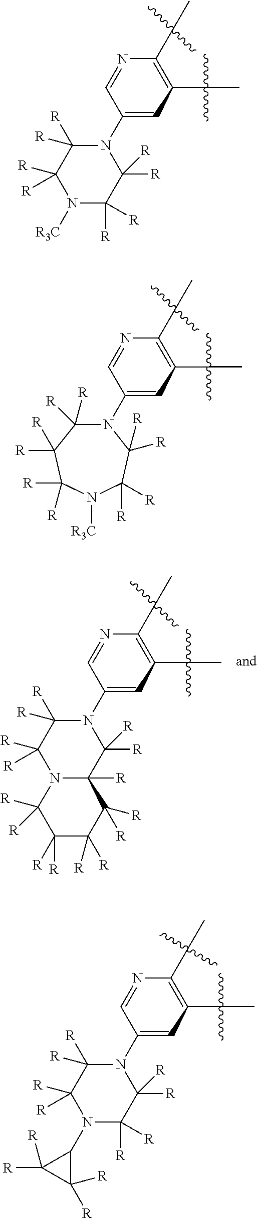

deuteration of similar R¹-type moieties, wherein the definition of R¹ is found herein. Deuteration of moieties within substituent W in compounds of the present disclosure are also contemplated, where W is defined herein (see, e.g., Formula Ib and subformulas thereof as well as specific examples of W described herein. Moreover, deuteration of available positions in any A moiety of compounds of the Formulas described herein is also contemplated. Further, deuterium substitution may also take place in compounds of the present disclosure at the linker position.

In a further embodiment, silylation substitution is also contemplated, such as in the linker as follows:

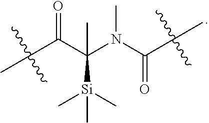 [SMILES: *C(=O)N(C)[C@@](C)(C(*)=O)[Si](C)(C)C]

As is known in the art, many chemical entities can adopt a variety of different solid forms such as, for example, amorphous forms or crystalline forms (e.g., polymorphs, hydrates, solvate). In some embodiments, compounds of the present disclosure may be utilized in any such form, including in any solid form. In some embodiments, compounds described or depicted herein may be provided or utilized in hydrate or solvate form.

At various places in the present specification, substituents of compounds of the present disclosure are disclosed in groups or in ranges. It is specifically intended that the present disclosure include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. Furthermore, where a compound includes a plurality of positions at which substituents are disclosed in groups or in ranges, unless otherwise indicated, the present disclosure is intended to cover individual compounds and groups of compounds (e.g., genera and subgenera) containing each and every individual subcombination of members at each position.

The term “optionally substituted X” (e.g., “optionally substituted alkyl”) is intended to be equivalent to “X, wherein X is optionally substituted” (e.g., “alkyl, wherein said alkyl is optionally substituted”). It is not intended to mean that the feature “X” (e.g., alkyl) per se is optional. As described herein, certain compounds of interest may contain one or more “optionally substituted” moieties. In general, the term “substituted”, whether preceded by the term “optionally” or not, means that one or more hydrogens of the designated moiety are replaced with a suitable substituent, e.g., any of the substituents or groups described herein. Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, in the term “optionally substituted C₁-C₆ alkyl-C₂-C₉ heteroaryl,” the alkyl portion, the heteroaryl portion, or both, may be optionally substituted. Combinations of substituents envisioned by the present disclosure are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group may be, independently, deuterium; halogen; —(CH₂)₀₋₄R^{∘}; —(CH₂)₀₋₄OR^{∘}; —O(CH₂)₀₋₄R^{∘}; —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; 4-8 membered saturated or unsaturated heterocycloalkyl (e.g., pyridyl); 3-8 membered saturated or unsaturated cycloalkyl (e.g., cyclopropyl, cyclobutyl, or cyclopentyl); —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄—C(O)—N(R^{∘})₂; —(CH₂)₀₋₄—C(O)—N(R^{∘})—S(O)₂—R^{∘}; —C(NCN)NR^{∘}₂; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)_{0- 4}OC(O) NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)_{0- 4}S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NOR^{∘})NR^{∘}₂; —C(NH)NR^{∘}₂; —P(O)₂R^{∘}; —P(O)R^{∘}₂; —P(O)(OR^{∘})₂; —OP(O)R^{∘}₂; —OP(O)(OR^{∘})₂; —OP(O)(OR^{∘})R^{∘}, —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, —C₁₋₆ aliphatic, —CH₂Ph, —O(CH₂)₀₋₁Ph, —CH₂-(5-6 membered heteroaryl ring), or a 3-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), may be, independently, halogen, —(CH₂)₀₋₂R^{•}, -(haloR^{•}), —(CH₂)₀₋₂OH, —(CH₂)₀₋₂₀R^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)_{0- 2}NR^{•}₂, —NO₂, —SiR^{•}₃, —OSiR^{•}₃, —C(O)SR^{•}, —(C₁₋₄ straight or branched alkylene)C(O)OR*, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on an aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{†} include ═O and ═S.

Those of ordinary skill in the art, reading the present disclosure, will appreciate that certain compounds described herein may be provided or utilized in any of a variety of forms such as, for example, salt forms, protected forms, pro-drug forms, ester forms, isomeric forms (e.g., optical or structural isomers), isotopic forms, etc. In some embodiments, reference to a particular compound may relate to a specific form of that compound. In some embodiments, reference to a particular compound may relate to that compound in any form. In some embodiments, for example, a preparation of a single stereoisomer of a compound may be considered to be a different form of the compound than a racemic mixture of the compound; a particular salt of a compound may be considered to be a different form from another salt form of the compound; a preparation containing one conformational isomer ((Z) or (E)) of a double bond may be considered to be a different form from one containing the other conformational isomer ((E) or (Z)) of the double bond; a preparation in which one or more atoms is a different isotope than is present in a reference preparation may be considered to be a different form.

As used herein, the term “administration” refers to the administration of a composition (e.g., a compound, or a preparation that includes a compound as described herein) to a subject or system.

Administration also includes administering a prodrug derivative or analog of the compound or pharmaceutically acceptable salt of the compound or composition to the subject, which can form an equivalent amount of active compound within the subject's body. Administration to an animal subject (e.g., to a human) may be by any appropriate route. For example, in some embodiments, administration may be bronchial (including by bronchial instillation), buccal, enteral, interdermal, intra-arterial, intradermal, intragastric, intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, or vitreal.

The term “acetyl,” as used herein, refers to the group —C(O)CH₃.

The term “alkoxy,” as used herein, refers to a —O—C₁-C₂₀ alkyl group, wherein the alkoxy group is attached to the remainder of the compound through an oxygen atom.

The term “alkyl,” as used herein, refers to a saturated, straight or branched monovalent hydrocarbon group containing from 1 to 20 (e.g., from 1 to 10 or from 1 to 6) carbons. In some embodiments, an alkyl group is unbranched (i.e., is linear); in some embodiments, an alkyl group is branched. Alkyl groups are exemplified by, but not limited to, methyl, ethyl, n- and iso-propyl, n-, sec-, iso- and tert-butyl, and neopentyl.

The term “alkylene,” as used herein, represents a saturated divalent hydrocarbon group derived from a straight or branched chain saturated hydrocarbon by the removal of two hydrogen atoms, and is exemplified by methylene, ethylene, isopropylene, and the like. The term “Cₓ-C_{y} alkylene” represents alkylene groups having between x and y carbons. Exemplary values for x are 1, 2, 3, 4, 5, and 6, and exemplary values for y are 2, 3, 4, 5, 6, 7, 8, 9, 10, 12, 14, 16, 18, or 20 (e.g., C₁-C₆, C₁-C₁₀, C₂-C₂₀, C₂-C₆, C₂-C₁₀, or C₂-C₂₀ alkylene). In some embodiments, the alkylene can be further substituted with 1, 2, 3, or 4 substituent groups as defined herein.

The term “alkenyl,” as used herein, represents monovalent straight or branched chain groups of, unless otherwise specified, from 2 to 20 carbons (e.g., from 2 to 6 or from 2 to 10 carbons) containing one or more carbon-carbon double bonds and is exemplified by ethenyl, 1-propenyl, 2-propenyl, 2-methyl-1-propenyl, 1-butenyl, and 2-butenyl. Alkenyls include both cis and trans isomers. The term “alkenylene,” as used herein, represents a divalent straight or branched chain groups of, unless otherwise specified, from 2 to 20 carbons (e.g., from 2 to 6 or from 2 to 10 carbons) containing one or more carbon-carbon double bonds.

The term “alkynyl,” as used herein, represents monovalent straight or branched chain groups from 2 to 20 carbon atoms (e.g., from 2 to 4, from 2 to 6, or from 2 to 10 carbons) containing a carbon-carbon triple bond and is exemplified by ethynyl, and 1-propynyl.

The term “amino,” as used herein, represents —N(R^{†})₂, e.g., —NH₂ and —N(CH₃)₂.

The term “aminoalkyl,” as used herein, represents an alkyl moiety substituted on one or more carbon atoms with one or more amino moieties.

The term “amino acid,” as described herein, refers to a molecule having a side chain, an amino group, and an acid group (e.g., —CO₂H or —SO₃H), wherein the amino acid is attached to the parent molecular group by the side chain, amino group, or acid group (e.g., the side chain). As used herein, the term “amino acid” in its broadest sense, refers to any compound or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. Exemplary amino acids include alanine, arginine, asparagine, aspartic acid, cysteine, glutamic acid, glutamine, glycine, histidine, optionally substituted hydroxylnorvaline, isoleucine, leucine, lysine, methionine, norvaline, ornithine, phenylalanine, proline, pyrrolysine, selenocysteine, serine, taurine, threonine, tryptophan, tyrosine, and valine.

An “amino acid substitution,” as used herein, refers to the substitution of a wild-type amino acid of a protein with a non-wild-type amino acid. Amino acid substitutions can result from genetic mutations and may alter one or more properties of the protein (e.g., may confer altered binding affinity or specificity, altered enzymatic activity, altered structure, or altered function).

The term “aryl,” as used herein, represents a monovalent monocyclic, bicyclic, or multicyclic ring system formed by carbon atoms, wherein the ring attached to the pendant group is aromatic. Examples of aryl groups are phenyl, naphthyl, phenanthrenyl, and anthracenyl. An aryl ring can be attached to its pendant group at any heteroatom or carbon ring atom that results in a stable structure and any of the ring atoms can be optionally substituted unless otherwise specified.

The term “C₀,” as used herein, represents a bond. For example, part of the term —N(C(O)—(C₀-C₅ alkylene-H)— includes —N(C(O)—(C₀ alkylene-H)—, which is also represented by —N(C(O)—H)—.

The terms “carbocyclic” and “carbocyclyl,” as used herein, refer to a monovalent, optionally substituted C₃-C₁₂ monocyclic, bicyclic, or tricyclic ring structure, which may be bridged, fused or spirocyclic, in which all the rings are formed by carbon atoms and at least one ring is non-aromatic. Carbocyclic structures include cycloalkyl, cycloalkenyl, and cycloalkynyl groups. Examples of carbocyclyl groups are cyclohexyl, cyclohexenyl, cyclooctynyl, 1,2-dihydronaphthyl, 1,2,3,4-tetrahydronaphthyl, fluorenyl, indenyl, indanyl, decalinyl, and the like. A carbocyclic ring can be attached to its pendant group at any ring atom that results in a stable structure and any of the ring atoms can be optionally substituted unless otherwise specified.

The term “carbonyl,” as used herein, represents a C(O) group, which can also be represented as C═O.

The term “carboxyl,” as used herein, means —CO₂H, (C═O)(OH), COOH, or C(O)OH or the unprotonated counterparts.

The term “combination therapy” refers to a method of treatment including administering to a subject at least two therapeutic agents, optionally as one or more pharmaceutical compositions, as part of a therapeutic regimen. For example, a combination therapy may include administration of a single pharmaceutical composition including at least two therapeutic agents and one or more pharmaceutically acceptable carrier, excipient, diluent, or surfactant. A combination therapy may include administration of two or more pharmaceutical compositions, each composition including one or more therapeutic agent and one or more pharmaceutically acceptable carrier, excipient, diluent, or surfactant. In various embodiments, at least one of the therapeutic agents is a RAS(ON) GTP hydrolysis-promoting compound (e.g., any one or more such RAS(ON) GTP hydrolysis-promoting compounds disclosed herein or known in the art). In various embodiments, at least one of the therapeutic agents is a KRAS(OFF) inhibitor (e.g., any one or more KRAS(OFF) inhibitor disclosed herein or known in the art). In some embodiments, at least one of the therapeutic agents is a KRAS^{G12C}(OFF) inhibitor (e.g., any one or more of the KRAS^{G12C}(OFF) inhibitors disclosed herein or known in the art). In some embodiments, at least one of the therapeutic agents is a KRAS^{G12D}(OFF) inhibitor (e.g., any one or more of the KRAS^{G12D} inhibitors disclosed herein or known in the art). In some embodiments, at least one of the therapeutic agents is a KRAS^{G12V}(OFF) inhibitor (e.g., any one or more of the KRAS^{G12V} inhibitors disclosed herein or known in the art). In some embodiments, at least one of the therapeutic agents is a pan-RAS(OFF) inhibitor (e.g., any one or more of the pan-RAS(OFF) inhibitors disclosed herein or known in the art). The two or more agents may optionally be administered simultaneously (as a single or as separate compositions) or sequentially (as separate compositions). The therapeutic agents may be administered in an effective amount. The therapeutic agent may be administered in a therapeutically effective amount. In some embodiments, the effective amount of one or more of the therapeutic agents may be lower when used in a combination therapy than the therapeutic amount of the same therapeutic agent when it is used as a monotherapy, e.g., due to an additive or synergistic effect of combining the two or more therapeutics.

The term “cyano,” as used herein, represents a —CN group.

The term “cycloalkyl,” as used herein, represents a monovalent saturated cyclic hydrocarbon group, which may be bridged, fused or spirocyclic having from three to eight ring carbons, unless otherwise specified, and is exemplified by cyclopropyl, cyclobutyl, cyclopentyl, cyclohexyl, cycloheptyl, and cycloheptyl.

The term “cycloalkenyl,” as used herein, represents a monovalent, non-aromatic, saturated cyclic hydrocarbon group, which may be bridged, fused or spirocyclic having from three to eight ring carbons, unless otherwise specified, and containing one or more carbon-carbon double bonds.

The term “diastereomer,” as used herein, means stereoisomers that are not mirror images of one another and are non-superimposable on one another.

As used herein, the term “dosage form” refers to a physically discrete unit of a compound (e.g., a compound of the present disclosure) for administration to a subject. Each unit contains a predetermined quantity of compound. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or compound administered to a particular subject is determined by one or more attending physicians and may involve administration of multiple dosage forms.

As used herein, the term “dosing regimen” refers to a set of unit doses (typically more than one) that are administered individually to a subject, typically separated by periods of time. In some embodiments, a given therapeutic compound (e.g., a compound of the present disclosure) has a recommended dosing regimen, which may involve one or more doses. In some embodiments, a dosing regimen includes a plurality of doses each of which are separated from one another by a time period of the same length; in some embodiments, a dosing regimen includes a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen includes a first dose in a first dose amount, followed by one or more additional doses in a second dose amount different from the first dose amount. In some embodiments, a dosing regimen includes a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

The term “disorder” is used in this disclosure to mean, and is used interchangeably with, the terms disease, condition, or illness, unless otherwise indicated.

The term “enantiomer,” as used herein, means each individual optically active form of a compound of the disclosure, having an optical purity or enantiomeric excess (as determined by methods standard in the art) of at least 80% (i.e., at least 90% of one enantiomer and at most 10% of the other enantiomer), preferably at least 90% and more preferably at least 98%.

The term “guanidinyl,” refers to a group having the structure:

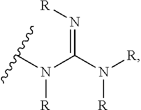

wherein each R is, independently, any chemically feasible substituent described herein.

The term “guanidinoalkyl alkyl,” as used herein, represents an alkyl moiety substituted on one or more carbon atoms with one or more guanidinyl moieties.

The term “haloacetyl,” as used herein, refers to an acetyl group wherein at least one of the hydrogens has been replaced by a halogen.

The term “haloalkyl,” as used herein, represents an alkyl moiety substituted on one or more carbon atoms with one or more of the same of different halogen moieties.

The term “halogen,” as used herein, represents a halogen selected from bromine, chlorine, iodine, or fluorine.

The term “heteroalkyl,” as used herein, refers to an “alkyl” group, as defined herein, in which at least one carbon atom has been replaced with a heteroatom (e.g., an O, N, or S atom). The heteroatom may appear in the middle or at the end of the radical.

The term “heteroaryl,” as used herein, represents a monovalent, monocyclic or polycyclic ring structure that contains at least one fully aromatic ring: i.e., they contain 4n+2 pi electrons within the monocyclic or polycyclic ring system and contains at least one ring heteroatom selected from N, O, or S in that aromatic ring. Exemplary unsubstituted heteroaryl groups are of 1 to 12 (e.g., 1 to 11, 1 to 10, 1 to 9, 2 to 12, 2 to 11, 2 to 10, or 2 to 9) carbons. The term “heteroaryl” includes bicyclic, tricyclic, and tetracyclic groups in which any of the above heteroaromatic rings is fused to one or more, aryl or carbocyclic rings, e.g., a phenyl ring, or a cyclohexane ring. Examples of heteroaryl groups include, but are not limited to, pyridyl, pyrazolyl, benzooxazolyl, benzoimidazolyl, benzothiazolyl, imidazolyl, thiazolyl, quinolinyl, tetrahydroquinolinyl, and 4-azaindolyl. A heteroaryl ring can be attached to its pendant group at any ring atom that results in a stable structure and any of the ring atoms can be optionally substituted unless otherwise specified. In some embodiments, the heteroaryl is substituted with 1, 2, 3, or 4 substituents groups.

The term “heterocycloalkyl,” as used herein, represents a monovalent monocyclic, bicyclic or polycyclic ring system, which may be bridged, fused or spirocyclic, wherein at least one ring is non-aromatic and wherein the non-aromatic ring contains one, two, three, or four heteroatoms independently selected from the group consisting of nitrogen, oxygen, and sulfur. The 5-membered ring has zero to two double bonds, and the 6- and 7-membered rings have zero to three double bonds. Exemplary unsubstituted heterocycloalkyl groups are of 1 to 12 (e.g., 1 to 11, 1 to 10, 1 to 9, 2 to 12, 2 to 11, 2 to 10, or 2 to 9) carbons. The term “heterocycloalkyl” also represents a heterocyclic compound having a bridged multicyclic structure in which one or more carbons or heteroatoms bridges two non-adjacent members of a monocyclic ring, e.g., a quinuclidinyl group. The term “heterocycloalkyl” includes bicyclic, tricyclic, and tetracyclic groups in which any of the above heterocyclic rings is fused to one or more aromatic, carbocyclic, heteroaromatic, or heterocyclic rings, e.g., an aryl ring, a cyclohexane ring, a cyclohexene ring, a cyclopentane ring, a cyclopentene ring, a pyridine ring, or a pyrrolidine ring. Examples of heterocycloalkyl groups are pyrrolidinyl, piperidinyl, 1,2,3,4-tetrahydroquinolinyl, decahydroquinolinyl, dihydropyrrolopyridine, and decahydronapthyridinyl. A heterocycloalkyl ring can be attached to its pendant group at any ring atom that results in a stable structure and any of the ring atoms can be optionally substituted unless otherwise specified.

The term “hydroxy,” as used herein, represents a —OH group.

The term “hydroxyalkyl,” as used herein, represents an alkyl moiety substituted on one or more carbon atoms with one or more —OH moieties.

The term “isomer,” as used herein, means any tautomer, stereoisomer, atropiosmer, enantiomer, or diastereomer of any compound of the disclosure. It is recognized that the compounds of the disclosure can have one or more chiral centers or double bonds and, therefore, exist as stereoisomers, such as double-bond isomers (i.e., geometric E/Z isomers) or diastereomers (e.g., enantiomers (i.e., (+) or (−)) or cis/trans isomers). According to the disclosure, the chemical structures depicted herein, and therefore the compounds of the disclosure, encompass all the corresponding stereoisomers, that is, both the stereomerically pure form (e.g., geometrically pure, enantiomerically pure, or diastereomerically pure) and enantiomeric and stereoisomeric mixtures, e.g., racemates. Enantiomeric and stereoisomeric mixtures of compounds of the disclosure can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein, the term “inhibitor” refers to a compound that prevents a biomolecule, (e.g., a protein, nucleic acid) from completing or initiating a reaction. An inhibitor can inhibit a reaction by competitive, uncompetitive, or non-competitive means, for example. With respect to its binding mechanism, an inhibitor may be an irreversible inhibitor or a reversible inhibitor. Exemplary inhibitors include, but are not limited to, nucleic acids, DNA, RNA, shRNA, siRNA, proteins, protein mimetics, peptides, peptidomimetics, antibodies, small molecules, chemicals, analogs that mimic the binding site of an enzyme, receptor, or other protein. In some embodiments, the inhibitor is a small molecule, e.g., a low molecular weight organic compound, e.g., an organic compound having a molecular weight (MW) of less than 1200 Daltons (Da). In some embodiments, the MW is less than 1100 Da. In some embodiments, the MW is less than 1000 Da. In some embodiments, the MW is less than 900 Da. In some embodiments, the MW is less than 800 Da. In some embodiments, the MW is less than 700 Da. In some embodiments, the MW is less than 600 Da. In some embodiments, the range of the MW of the small molecule is between 600 Da and 700 Da, inclusive. In some embodiments, the range of the MW of the small molecule is between 600 Da and 800 Da, inclusive. Small molecule inhibitors include cyclic and acyclic compounds. Small molecules inhibitors include natural products, derivatives, and analogs thereof. Small molecule inhibitors can include a covalent cross-linking group capable of forming a covalent crosslink, e.g., with an amino acid side-chain of a target protein.

As used herein, the term “linker” refers to a divalent organic moiety connecting a first moiety (e.g., a macrocyclic moiety) to a second moiety (e.g., a cross-linking group). In some embodiments, the linker results in a compound capable of achieving an IC50 of 2 uM or less in the Ras-RAF disruption assay protocol provided here:

The purpose of this biochemical assay is to measure the ability of test compounds to facilitate ternary complex formation between a nucleotide-loaded Ras isoform and cyclophilin A; the resulting ternary complex disrupts binding to a BRAF^{RBD} construct, inhibiting Ras signaling through a RAF effector.

In assay buffer containing 25 mM HEPES pH 7.3, 0.002% Tween20, 0.1% BSA, 100 mM NaCl and 5 mM MgCl₂, tagless Cyclophilin A, His6-K-Ras-GMPPNP (or other Ras variant), and GST-BRAF^{RBD} are combined in a 384-well assay plate at final concentrations of 25 μM, 12.5 nM and 50 nM, respectively. Compound is present in plate wells as a 10-point 3-fold dilution series starting at a final concentration of 30 μM. After incubation at 25° C. for 3 hours, a mixture of Anti-His Eu-W1024 and anti-GST allophycocyanin is then added to assay sample wells at final concentrations of 10 nM and 50 nM, respectively, and the reaction incubated for an additional 1.5 hours. TR-FRET signal is read on a microplate reader (Ex 320 nm, Em 665/615 nm). Compounds that facilitate disruption of a Ras:RAF complex are identified as those eliciting a decrease in the TR-FRET ratio relative to DMSO control wells.

In some embodiments, the linker comprises 20 or fewer linear atoms. In some embodiments, the linker comprises 15 or fewer linear atoms. In some embodiments, the linker comprises 10 or fewer linear atoms. In some embodiments, the linker has a molecular weight of under 500 g/mol. In some embodiments, the linker has a molecular weight of under 400 g/mol. In some embodiments, the linker has a molecular weight of under 300 g/mol. In some embodiments, the linker has a molecular weight of under 200 g/mol. In some embodiments, the linker has a molecular weight of under 100 g/mol. In some embodiments, the linker has a molecular weight of under 50 g/mol.

The term “mutation” as used herein indicates any modification of a nucleic acid or polypeptide which results in an altered nucleic acid or polypeptide. The term “mutation” may include, for example, point mutations, deletions or insertions of single or multiple residues in a polynucleotide, which includes alterations arising within a protein-encoding region of a gene as well as alterations in regions outside of a protein-encoding sequence, such as, but not limited to, regulatory or promoter sequences, as well as amplifications or chromosomal breaks or translocations. In particular embodiments, the mutation results in an amino acid substitution in the encoded-protein.

A “patient” or “subject” is a mammal, e.g., a human, mouse, rat, guinea pig, dog, cat, horse, cow, pig, or non-human primate, such as a monkey, chimpanzee, baboon or rhesus.

The term “prevent” or “preventing” with regard to a subject refers to keeping a disease or disorder from afflicting the subject. Preventing includes prophylactic treatment. For instance, preventing can include administering to the subject a compound disclosed herein before a subject is afflicted with a disease and the administration will keep the subject from being afflicted with the disease.

As used herein, the term “pharmaceutical composition” refers to a compound, such as a compound of the present disclosure, or a pharmaceutically acceptable salt thereof, formulated together with a pharmaceutically acceptable excipient.

A “pharmaceutically acceptable excipient,” as used herein, refers any inactive ingredient (for example, a vehicle capable of suspending or dissolving the active compound) having the properties of being nontoxic and non-inflammatory in a subject. Typical excipients include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Excipients include, but are not limited to: butylated optionally substituted hydroxyltoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, optionally substituted hydroxylpropyl cellulose, optionally substituted hydroxylpropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol. Those of ordinary skill in the art are familiar with a variety of agents and materials useful as excipients. See, e.g., Ansel, et al., Ansel's Pharmaceutical Dosage Forms and Drug Delivery Systems. Philadelphia: Lippincott, Williams & Wilkins, 2004; Gennaro, et al., Remington: The Science and Practice of Pharmacy. Philadelphia: Lippincott, Williams & Wilkins, 2000; and Rowe, Handbook of Pharmaceutical Excipients. Chicago, Pharmaceutical Press, 2005. In some embodiments, a composition includes at least two different pharmaceutically acceptable excipients.

The term “pharmaceutically acceptable salt,” as use herein, refers to those salts of the compounds described herein that are, within the scope of sound medical judgment, suitable for use in contact with the tissues of humans and other animals without undue toxicity, irritation, allergic response and the like, and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The terms “RAS inhibitor” and “inhibitor of [a] RAS” are used interchangeably to refer to any inhibitor that targets, that is, selectively binds to or inhibits a RAS protein.

As used herein, the term “RAS(OFF) inhibitor” refers to an inhibitor that targets, that is, selectively binds to or inhibits the GDP-bound, inactive state of RAS (e.g., selective over the GTP-bound, active state of RAS). Inhibition of the GDP-bound, inactive state of RAS includes, for example, sequestering the inactive state by inhibiting the exchange of GDP for GTP, thereby inhibiting RAS from adopting the active conformation. In certain embodiments, RAS(OFF) inhibitors may also bind to or inhibit the GTP-bound, active state of RAS (e.g., with a lower affinity or inhibition constant than for the GDP-bound, inactive state of RAS). A RAS(OFF) inhibitor may be mutant-selective, such as selective for a G12C, G12D or G12V mutant. A RAS(OFF) inhibitor may be selective for more than one mutant, or selective for one or more mutants and for wild-type (in either situation, a “pan-RAS(OFF)” inhibitor). Methods of measuring RAS(OFF) inhibition are known in the art.

As used herein, the term “RAS(ON) inhibitor” refers to a non-covalent tri-complex-forming inhibitor that targets, that is, selectively binds to or inhibits, the GTP-bound, active state of RAS (e.g., selective over the GDP-bound, inactive state of RAS). Inhibition of the GTP-bound, active state of RAS includes, for example, the inhibition of oncogenic signaling from the GTP-bound, active state of RAS. In some embodiments, the RAS(ON) inhibitor is an inhibitor that selectively binds to and inhibits the GTP-bound, active state of RAS. In certain embodiments, RAS(ON) inhibitors may also bind to or inhibit the GDP-bound, inactive state of RAS (e.g., with a lower affinity or inhibition constant than for the GTP-bound, active state of RAS). RAS(ON) inhibitors are non-covalent binders of the GTP-bound form of RAS, and persons of skill in the art are familiar with methods of determining if crosslinking has occurred. In some embodiments, a RAS(ON) inhibitor does not comprise a crosslinking group, such as crosslinking groups found in the art (e.g., WO 2020/132597, WO 2021/091982, WO 2021/091967, WO 2022/235864, WO 2022/235870, WO 2023/060253, PCT/US2023/037057 and WO 2023/133543). In some embodiments, a RAS(ON) inhibitor has a molecular weight of between 800 and 1200 Da, inclusive. Reference to the term RAS(ON) inhibitor includes, without limitation, any one or more RAS(ON) inhibitors selected from the RAS(ON) inhibitors disclosed in WO 2021/091956, WO 2022/060836, U.S. provisional application Ser. No. 63/351,146, or WO 2023/240263, each of which is incorporated by reference in its entirety, or a combination of any such RAS(ON) inhibitors. In some embodiments, compounds from WO 2021/091956, WO 2022/060836 and WO 2023/240263, comprising a phenol at the A position of each is excluded. Methods of determining RAS(ON) inhibition are known in the art. See, e.g., WO 2021/091956, WO 2022/060836 and WO 2023/240263.

As used herein, a “RAS(ON) GTP hydrolysis-promoting compound” refers to a tri-complex-forming compound that, when bound in a tri-complex (i.e. CYPA-RAS(ON) GTP hydrolysis-promoting compound-RAS(ON) isoform), exhibits a RAS(ON) GTP hydrolysis rate that is greater than the intrinsic hydrolysis rate of the RAS(ON) mutant isoform (RAS^{MUT}) and/or RAS wild-type isoform (RAS^{WT}) in the absence of the compound. In some embodiments, a RAS(ON) GTP hydrolysis-promoting compound exhibits a hydrolysis rate that is greater than 14× the intrinsic hydrolysis rate (a “strong hydrolyzer”). In some embodiments, a RAS(ON) GTP-hydrolysis-promoting compound exhibits a hydrolysis rate that is 5-14× the intrinsic hydrolysis rate (a “moderate hydrolyzer”). In some embodiments, a RAS(ON) GTP hydrolysis-promoting compound exhibits a hydrolysis rate that is greater than 1× the intrinsic hydrolysis rate but less than 5× the intrinsic hydrolysis rate (a “weak hydrolyzer”). In some embodiments, KRAS^{G12V} is the isoform used for determining a strong hydrolyzer, moderate hydrolyzer, or weak hydrolyzer.

Methods of measuring hydrolysis are known in the art, such as those described herein. In some embodiments, a RAS(ON) GTP hydrolysis-promoting compound is a RAS(ON) inhibitor. All RAS(ON) GTP hydrolysis-promoting compounds retain a catalytic water in the proximity of the gamma phosphorous of GTP (distance<5 angstroms) and position the delta carbon of the Q61 of RAS(ON) within 8 angstroms of the gamma phosphorous: these parameters may be determined by one of skill in the art. Further description of RAS(ON) GTP hydrolysis-promoting compounds are described herein.

As used herein, a “RAS(OFF) degrader” is a RAS degrader that targets the OFF state. Such degraders are known in the art. Non-limiting examples of RAS(OFF) degraders may be found in one or more of the following applications: WO 2024055112, WO 2024054625, WO 2024050742, WO 2024044334, WO 2024040080, WO 2024034657, WO 2024034593, WO 2024034591, WO 2024034123, WO 2024029613, WO 2024020159, WO 2024019103, WO 2024017392, WO 2023185864, WO 2023171781, WO 2023141570, WO 2023138524, WO 2023130012, WO 2023116934, WO 2023099620, WO 2023081476, WO 2023077441, and CN 115785199, each of which is incorporated herein by reference in its entirety.

The terms “RAS pathway” and “RAS/MAPK pathway” are used interchangeably herein to refer to a signal transduction cascade downstream of various cell surface growth factor receptors in which activation of RAS (and its various isoforms and allotypes) is a central event that drives a variety of cellular effector events that determine the proliferation, activation, differentiation, mobilization, and other functional properties of the cell. SHP2 conveys positive signals from growth factor receptors to the RAS activation/deactivation cycle, which is modulated by guanine nucleotide exchange factors (GEFs, such as SOS1) that load GTP onto RAS to produce functionally active GTP-bound RAS as well as GTPase-activating protein (GAPs, such as NF1) that facilitate termination of the signals by conversion of GTP to GDP. GTP-bound RAS produced by this cycle conveys essential positive signals to a series of serine/threonine kinases including RAF and MAP kinases, from which emanate additional signals to various cellular effector functions.

The term “stereoisomer,” as used herein, refers to all possible different isomeric as well as conformational forms which a compound may possess (e.g., a compound of any formula described herein), in particular all possible stereochemically and conformationally isomeric forms, all diastereomers, enantiomers or conformers of the basic molecular structure, including atropisomers. Some compounds of the present disclosure may exist in different tautomeric forms, all of the latter being included within the scope of the present disclosure.

The term “sulfonyl,” as used herein, represents an —S(O)₂— group.

A “therapeutic agent” is any substance, e.g., a compound or composition, capable of treating a disease or disorder. In some embodiments, therapeutic agents that are useful in connection with the present disclosure include RAS inhibitors and cancer chemotherapeutics. Many such therapeutic agents are known in the art and are disclosed herein.

The term “therapeutically effective amount” means an amount that is sufficient, when administered to a population suffering from or susceptible to a disease, disorder, or condition in accordance with a therapeutic dosing regimen, to treat the disease, disorder, or condition. In some embodiments, a therapeutically effective amount is one that reduces the incidence or severity of, or delays onset of, one or more symptoms of the disease, disorder, or condition. Those of ordinary skill in the art will appreciate that the term “therapeutically effective amount” does not in fact require successful treatment be achieved in a particular individual. Rather, a therapeutically effective amount may be that amount that provides a particular desired pharmacological response in a significant number of subjects when administered to patients in need of such treatment. It is specifically understood that particular subjects may, in fact, be “refractory” to a “therapeutically effective amount.” In some embodiments, reference to a therapeutically effective amount may be a reference to an amount as measured in one or more specific tissues (e.g., a tissue affected by the disease, disorder or condition) or fluids (e.g., blood, saliva, serum, sweat, tears, urine). Those of ordinary skill in the art will appreciate that, in some embodiments, a therapeutically effective amount may be formulated or administered in a single dose. In some embodiments, a therapeutically effective amount may be formulated or administered in a plurality of doses, for example, as part of a dosing regimen.

The term “thiocarbonyl,” as used herein, refers to a —C(S)— group.

The term “treatment” (also “treat” or “treating”), in its broadest sense, refers to any administration of a substance (e.g., a compound of the present disclosure) that partially or completely alleviates, ameliorates, relieves, inhibits, delays onset of, reduces severity of, or reduces incidence of one or more symptoms, features, or causes of a particular disease, disorder, or condition. In some embodiments, such treatment may be administered to a subject who does not exhibit signs of the relevant disease, disorder or condition or of a subject who exhibits only early signs of the disease, disorder, or condition.

Alternatively, or additionally, in some embodiments, treatment may be administered to a subject who exhibits one or more established signs of the relevant disease, disorder or condition. In some embodiments, treatment may be of a subject who has been diagnosed as suffering from the relevant disease, disorder, or condition. In some embodiments, treatment may be of a subject known to have one or more susceptibility factors that are statistically correlated with increased risk of development of the relevant disease, disorder, or condition.

The term “tri-complex” refers to having a mechanism of action entailing formation of a high affinity three-component complex between a synthetic ligand (e.g., a RAS(ON) GTP hydrolysis-promoting compound) and two intracellular proteins which do not interact under normal physiological conditions: the target protein of interest, RAS, and a widely expressed cytosolic chaperone protein in the cell, cyclophilin A. Such tri-complexes are known in the art. See, e.g., WO 2020/132597, WO 2021/091956, WO 2021/091967, WO 2021/091982, WO 2022/060836, WO 2022/235864, WO 2022/235/870, WO 2023/060253, WO 2023/133543, and WO 2023/240263.

The term “wild-type” refers to an entity having a structure or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

### 1. Compositions

Provided herein are compounds that promote GTP hydrolysis of RAS(ON) and uses thereof. Also provided are pharmaceutical compositions including one or more such compounds, or a pharmaceutically acceptable salt thereof, and a pharmaceutically acceptable excipient. RAS(ON) GTP hydrolysis-promoting compounds may be used in methods of modulating RAS (e.g., in a subject or in a cell) and methods of treating cancer, as described herein. The present disclosure provides, inter alia, compositions, methods, and kits for treating or preventing a disease or disorder (e.g., cancer) with a RAS(ON) GTP hydrolysis-promoting compound in combination with a RAS(OFF) inhibitor.

RAS proteins (KRAS, HRAS, and NRAS) play an essential role in various human cancers and are therefore appropriate targets for anticancer therapy. Indeed, mutations in RAS proteins account for approximately 30% of all human cancers in the United States, many of which are fatal. Dysregulation of RAS proteins by activating mutations, overexpression, or upstream activation is common in human tumors, and activating mutations in RAS are frequently found in human cancer. RAS converts between a GDP-bound “off” and a GTP-bound “on” state. The conversion between states is facilitated by interplay between a guanine nucleotide exchange factor (GEF) protein (e.g., SOS1), which loads RAS with GTP, and a GTPase-activating protein (GAP) protein (e.g., NF1), which hydrolyzes GTP, thereby inactivating RAS. Additionally, the SH2 domain-containing protein tyrosine phosphatase-2 (SHP2) associates with the receptor signaling apparatus and becomes active upon RTK activation, and then promote RAS activation. Mutations in RAS proteins can lock the protein in the “on” state resulting in a constitutively active signaling pathway that leads to uncontrolled cell growth. For example, activating mutations at codon 12 in RAS proteins function by inhibiting both GAP-dependent and intrinsic hydrolysis rates of GTP, significantly skewing the population of RAS mutant proteins to the “on” (GTP-bound) state (RAS(ON)), leading to oncogenic MAPK signaling. Notably, RAS exhibits a picomolar affinity for GTP, enabling RAS to be activated even in the presence of low concentrations of this nucleotide. Mutations at codons 13 (e.g., G13D) and 61 (e.g., Q61K) of RAS are also responsible for oncogenic activity in some cancers.

### a) RAS(ON) GTP Hydrolysis-Promoting Compounds

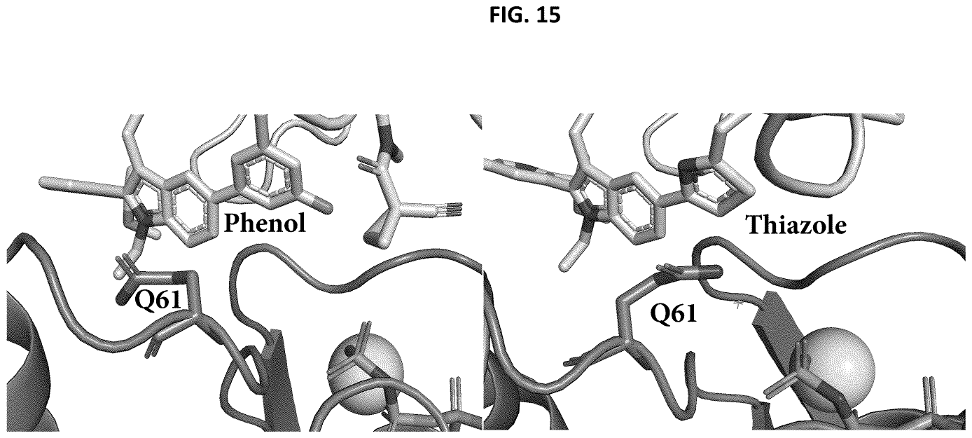

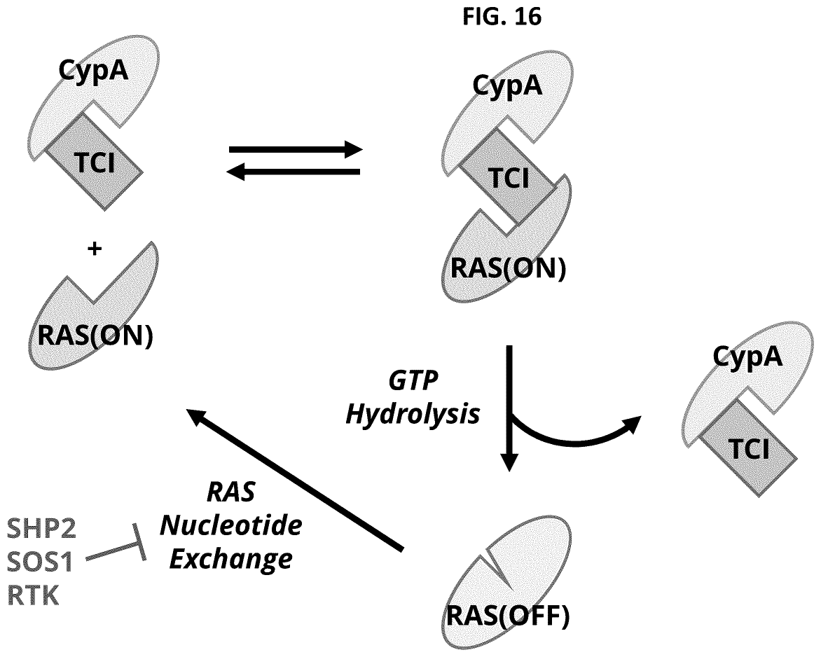

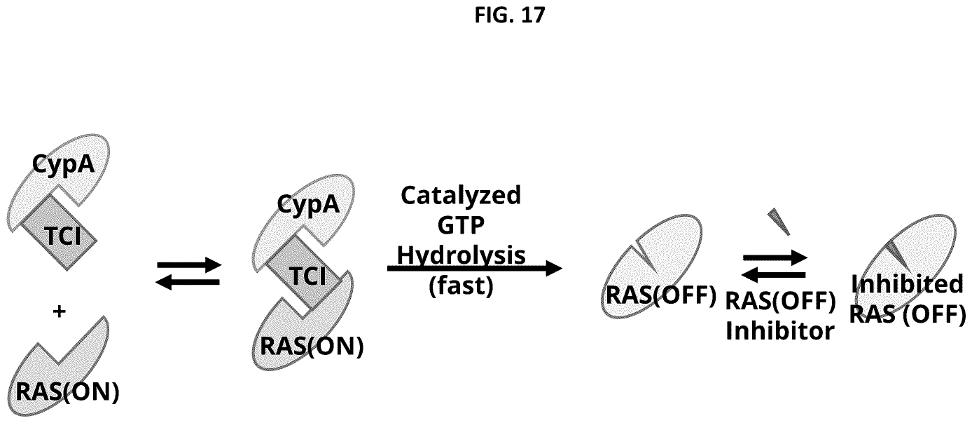

Compositions of the present disclosure may include one or more RAS(ON) GTP hydrolysis-promoting compounds. A RAS(ON) GTP hydrolysis-promoting compound of the present disclosure forms a high affinity tri-complex with two intracellular proteins which do not interact under normal physiological conditions: RAS and a widely expressed cytosolic chaperone in the cell, cyclophilin A (CypA). In addition, the present disclosure provides non-covalent binding of RAS by a RAS(ON) GTP hydrolysis-promoting compound to promote a catalytically competent orientation of RAS(ON) in which the glutamine 61 (Q61) side chain coordinates a catalytic water to promote nucleophilic attack at the gamma phosphate GTP bound to the RAS protein. See FIG. 15.

Accordingly, provided herein is a RAS(ON) GTP hydrolysis-promoting compound, having the structure of Formula Ia:

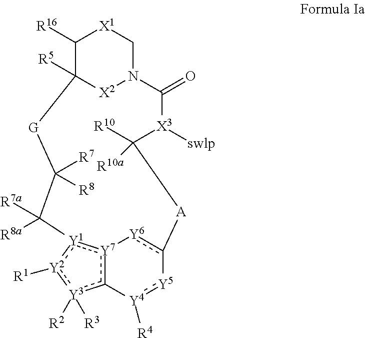

or a pharmaceutically acceptable salt thereof, wherein the dotted lines represent zero, one, two, three, or four non-adjacent double bonds;
A is optionally substituted C₂-C₄ alkylene, optionally substituted C₁-C₄ heteroalkylene, or optionally substituted C₂-C₄ alkenylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
swIp (Switch I/P-loop) is an organic moiety that non-covalently binds to, and does not crosslink to, both the Switch I binding pocket and residues 12 or 13 of the P-loop of a Ras protein (see, e.g., Johnson et al., 292:12981-12993 (2017), incorporated herein by reference);
X¹ is optionally substituted C₁-C₂ alkylene, NR, O, or S(O)n;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂; each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally 30 substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent, or
R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or
R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R^{8′}; C═N(OH), C═N(O—C₁-C₃ alkyl), C═O, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R⁷ is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R¹⁰ is hydrogen, halo, hydroxy, C₁-C₃ alkoxy, or C₁-C₃ alkyl;
R^{10a} is hydrogen or halo; and
R¹⁶ is hydrogen or C₁-C₃ alkyl.

In some embodiments, the disclosure features a compound of structural Formula Ib that does not crosslink to a RAS protein:

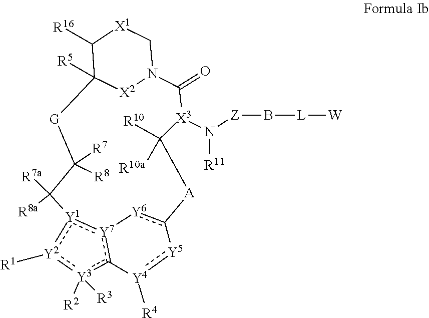

or a pharmaceutically acceptable salt thereof, wherein the dotted lines represent zero, one, two, three, or four non-adjacent double bonds;
A is optionally substituted C₂-C₄ alkylene, optionally substituted C₁-C₄ heteroalkylene, or optionally substituted C₂-C₄ alkenylene;
B is absent, —NH—, —N(CH₃)—, —O—, —CH(R⁹)— or >C═CR⁹R^{9′} where the carbon is bound to the carbonyl carbon of —N(R¹¹)C(O)—, optionally substituted 3 to 6-membered cycloalkylene, optionally substituted 3 to 6-membered heterocycloalkylene, optionally substituted 6-membered arylene, or 5 to 6-membered heteroarylene;
G is optionally substituted C₁-C₄ alkylene, optionally substituted C₁-C₄ alkenylene, optionally substituted C₁-C₄ heteroalkylene, —C(O)O—CH(R⁶)— where C is bound to —C(R⁷R⁸)—, —C(O)NH—CH(R⁶)— where C is bound to —C(R⁷R⁸)—, optionally substituted C₁-C₄ heteroalkylene, or 3 to 8-membered heteroarylene;
L is absent or a linker;
W is hydrogen, cyano, optionally substituted amino, optionally substituted amido, optionally substituted C₁-C₄ alkoxy, optionally substituted C₁-C₄ hydroxyalkyl, optionally substituted C₁-C₄ aminoalkyl, optionally substituted C₁-C₄ haloalkyl, optionally substituted C₁-C₄ alkyl, optionally substituted C₁-C₄ guanidinoalkyl, C₀-C₄ alkyl optionally substituted 3 to 11-membered heterocycloalkyl, optionally substituted 3 to 10-membered cycloalkyl, optionally substituted 6 to 10-membered aryl, or optionally substituted 3 to 10-membered heteroaryl, wherein W does not crosslink to RAS;
Z is —C(O)— or —S(O)₂—;
X¹ is optionally substituted C₁-C₂ alkylene, NR, O, or S(O)ₙ;
X² is O or NH;
X³ is N or CH;
n is 0, 1, or 2;
R is hydrogen, cyano, optionally substituted C₁-C₄ alkyl, optionally substituted C₂-C₄ alkenyl, optionally substituted C₂-C₄ alkynyl, C(O)R′, C(O)OR′, C(O)N(R′)₂, S(O)R′, S(O)₂R′, or S(O)₂N(R′)₂;
each R′ is, independently, H or optionally substituted C₁-C₄ alkyl;
Y¹ is C, CH, or N;
Y², Y³, Y⁴, and Y⁷ are, independently, C or N;
Y⁵ is CH, CH₂, or N;
Y⁶ is C(O), CH, CH₂, or N;
R¹ is cyano, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, optionally substituted C₁-C₆ alkyl, optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl; R³ is absent or R² and R³ combine with the atom to which they are attached to form an optionally substituted 3 to 8-membered cycloalkyl or optionally substituted 3 to 14-membered heterocycloalkyl;
R⁴ is absent, hydrogen, halogen, cyano, or methyl optionally substituted with 1 to 3 halogens;
R⁵ is hydrogen, C₁-C₄ alkyl optionally substituted with halogen, cyano, hydroxy, or C₁-C₄ alkoxy, cyclopropyl, or cyclobutyl;
R⁶ is hydrogen or methyl; R⁷ is hydrogen, halogen, or optionally substituted C₁-C₃ alkyl, or R⁶ and R⁷ combine with the carbon atoms to which they are attached to form an optionally substituted 3 to 6-membered cycloalkyl or optionally substituted 3 to 7-membered heterocycloalkyl;
R⁸ is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R⁷ and R⁸ combine with the carbon atom to which they are attached to form C═CR⁷′R^{8′}; C═N(OH), C═N(O—C₁-C₃ alkyl), C═0, C═S, C═NH, optionally substituted 3 to 6-membered cycloalkyl, or optionally substituted 3 to 7-membered heterocycloalkyl;
R^{7a} and R^{8a} are, independently, hydrogen, halo, optionally substituted C₁-C₃ alkyl, or combine with the carbon to which they are attached to form a carbonyl;
R^{7′} is hydrogen, halogen, or optionally substituted C₁-C₃ alkyl; R^{8′} is hydrogen, halogen, hydroxy, cyano, optionally substituted C₁-C₃ alkoxy, optionally substituted C₁-C₃ alkyl, optionally substituted C₂-C₆ alkenyl, optionally substituted C₂-C₆ alkynyl, optionally substituted 3 to 8-membered cycloalkyl, optionally substituted 3 to 14-membered heterocycloalkyl, optionally substituted 5 to 10-membered heteroaryl, or optionally substituted 6 to 10-membered aryl, or
R^{7′} and R^{8′} combine with the carbon atom to which they are attached to form optionally substituted 3 to 6-membered cycloalkyl or optionally substituted 3 to 7-membered heterocycloalkyl;
R⁹ is hydrogen, F, optionally substituted C₁-C₆ alkyl, optionally substituted C₁-C₆ heteroalkyl, optionally substituted 3 to 6-membered cycloalkyl, or optionally substituted 3 to 7-membered heterocycloalkyl;
R⁹ and L combine with the atoms to which they are attached to form an optionally substituted 3 to 14-membered heterocycloalkyl;
R^{9′} is hydrogen or optionally substituted C₁-C₆ alkyl;
R¹⁰ is hydrogen, halo, hydroxy, C₁-C₃ alkoxy, or C₁-C₃ alkyl;
R^{10a} is hydrogen or halo;
R¹¹ is hydrogen or C₁-C₃ alkyl; and
R¹⁶ is hydrogen or C₁-C₃ alkyl.

In some embodiments, the disclosure provides a compound of Formula Ia

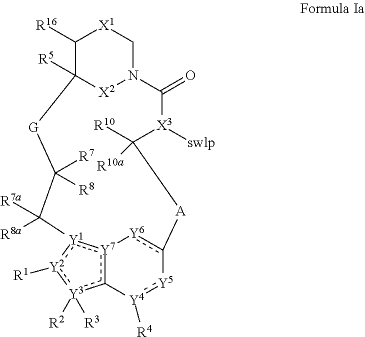

wherein R¹ is cyano, halogen (e.g., fluoro) optionally substituted C₁-C₆ alkyl (e.g., C₁-C₆ haloalkyl or C₁-C₆ fluoroalkyl) optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, halogen (e.g., fluoro), optionally substituted C₁-C₆ alkyl, C₁-C₆ haloalkyl (e.g., C₁-C₆ fluoroalkyl), optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent or R² and R³ combine with the atom to which they are attached to form an optionally substituted 3 to 8-membered cycloalkyl or optionally substituted 3 to 14-membered heterocycloalkyl;
R⁴ is absent, hydrogen, halogen (e.g., fluoro), cyano, or methyl optionally substituted with 1 to 3 halogens;
R⁵ is hydrogen, halogen (e.g., fluoro) C₁-C₄ alkyl optionally substituted with halogen (e.g., fluoro), cyano, hydroxy, or C₁-C₄ alkoxy, cyclopropyl, or cyclobutyl;
Y⁵ is CH, CH₂, CF, CHF, CF₂, or N;
Y⁶ is C(O), CH, CF, CH₂, CF₂, or N;
and the remaining variables are as defined above.

In some embodiments, the disclosure provides a compound of Formula Ib

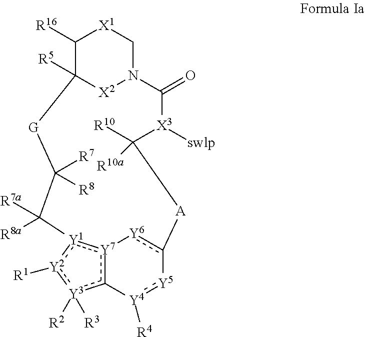

wherein R¹ is cyano, halogen (e.g., fluoro) optionally substituted C₁-C₆ alkyl (e.g., C₁-C₆ haloalkyl or C₁-C₆ fluoroalkyl) optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl, or
R¹ and R² combine with the atoms to which they are attached to form an optionally substituted 3 to 14-membered heterocycloalkyl;
R² is absent, hydrogen, halogen (e.g., fluoro), optionally substituted C₁-C₆ alkyl, C₁-C₆ haloalkyl (e.g., C₁-C₆ fluoroalkyl), optionally substituted C₂-C₆ alkenyl, optionally substituted C₂-C₆ alkynyl, optionally substituted 3 to 6-membered cycloalkyl, optionally substituted 3 to 7-membered heterocycloalkyl, optionally substituted 6-membered aryl, optionally substituted 5 or 6-membered heteroaryl;
R³ is absent or R² and R³ combine with the atom to which they are attached to form an optionally substituted 3 to 8-membered cycloalkyl or optionally substituted 3 to 14-membered heterocycloalkyl;
R⁴ is absent, hydrogen, halogen (e.g., fluoro), cyano, or methyl optionally substituted with 1 to 3 halogens;
R⁵ is hydrogen, halogen (e.g., fluoro) C₁-C₄ alkyl optionally substituted with halogen (e.g., fluoro), cyano, hydroxy, or C₁-C₄ alkoxy, cyclopropyl, or cyclobutyl;
Y⁵ is CH, CH₂, CF, CHF, CF₂, or N;
Y⁶ is C(O), CH, CF, CH₂, CF₂, or N;
and the remaining variables are as defined above.

In some embodiments, A is one of the following:

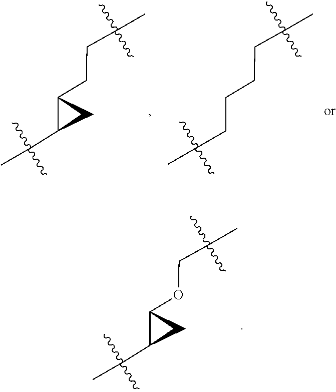 [SMILES: *CC[C@@H]1C[C@H]1* ; *CCCC* ; *CO[C@@H]1C[C@H]1*]

In some embodiments, A is one of the following:

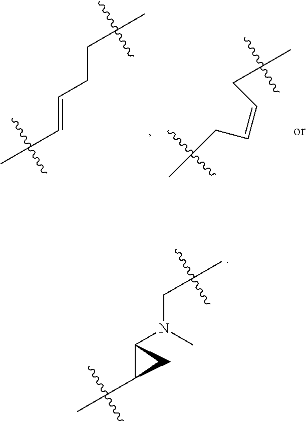

In some embodiments, R¹ is

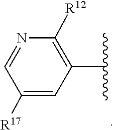

In some embodiments, R¹ is

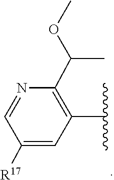

In some embodiments, R¹ is

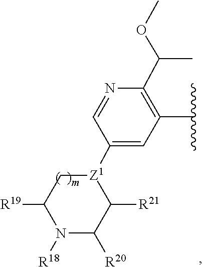

wherein Z¹ is N or CH;
m is 1 or 2
R¹⁸, R¹⁹, R²⁰, and R²¹ are each independently selected from hydrogen, optionally substituted C₁-C₆ alkyl, optionally substituted C₁-C₆ heteroalkyl, optionally substituted 3 to 6-membered cycloalkyl, optionally substituted 3 to 6-membered cycloalkenyl, optionally substituted 3 to 6-membered heterocycloalkyl, optionally substituted 6 to 10-membered aryl, or optionally substituted 5 to 10-membered heteroaryl; or
R¹⁸ and R²⁰ combine with the atoms to which they are attached to form an optionally substituted 3 to 8-membered cycloalkyl or an optionally substituted 3 to 8-membered heterocycloalkyl; or
R²⁰ and R²¹ combine with the atoms to which they are attached to form an optionally substituted 3 to 8-membered heterocycloalkyl; or
R¹⁹ and R²⁰ combine with the atoms to which they are attached to form an optionally substituted 4 to 8-membered heterocycloalkyl.

In some embodiments, R¹ is

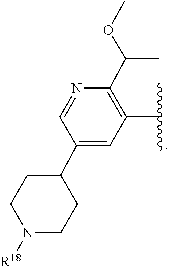

In some embodiments, R¹ is

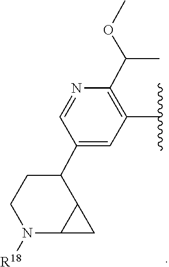

In some embodiments, R¹⁸ is methyl.

In some embodiments, R¹ is

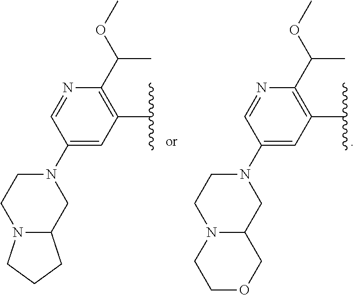 [SMILES: *c1cc(N2CCN3CCCC3C2)cnc1C(C)OC ; *c1cc(N2CCN3CCOCC3C2)cnc1C(C)OC]

In some embodiments, B is —CHR⁹—. In some embodiments, R⁹ is optionally substituted C₁-C₆ alkyl or optionally substituted 3 to 6-membered cycloalkyl. In some embodiments, B is optionally substituted 6-membered arylene. In some embodiments, B is absent.

In some embodiments, the linker has the structure has the structure of Formula II:

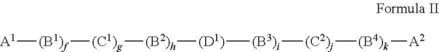

where A¹ is a bond between the linker and B; A² is a bond between W and the linker; B¹, B², B³, and B⁴ each, independently, is selected from optionally substituted C₁-C₂ alkylene, optionally substituted C₁-C₃ heteroalkylene, O, S, and NR^{N}; R^{N} is hydrogen, optionally substituted C₁-C₄ alkyl, optionally substituted C₁-C₃ cycloalkyl, optionally substituted C₂-C₄ alkenyl, optionally substituted C₂-C₄ alkynyl, optionally substituted 3 to 14-membered heterocycloalkyl, optionally substituted 6 to 10-membered aryl, or optionally substituted C₁-C₇ heteroalkyl; C¹ and C² are each, independently, selected from carbonyl, thiocarbonyl, sulphonyl, or phosphoryl; f, g, h, i, j, and k are each, independently, 0 or 1; and D¹ is optionally substituted C₁-C₁₀ alkylene, optionally substituted C₂-C₁₀ alkenylene, optionally substituted C₂-C₁₀ alkynylene, optionally substituted 3 to 14-membered heterocycloalkylene, optionally substituted 5 to 10-membered heteroarylene, optionally substituted 3 to 8-membered cycloalkylene, optionally substituted 6 to 10-membered arylene, optionally substituted C₂-C₁₀ polyethylene glycolene, or optionally substituted C₁-C₁₀ heteroalkylene, or a chemical bond linking A¹-(B¹)_{f}—(C¹)_{g}—(B²)ₕ— to —(B³)ᵢ—(C²)ⱼ—(B⁴)ₖ-A².

In some embodiments, the linker is acyclic. In some embodiments, the linker has the structure of Formula IIa:

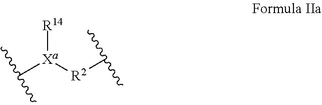

wherein X^{a} is absent or N;
R¹⁴ is absent, hydrogen, optionally substituted C₁-C₆ alkyl, or optionally substituted C₁-C₃ cycloalkyl; and
L² is absent, —C(O)—, —SO₂—, optionally substituted C₁-C₄ alkylene or optionally substituted C₁-C₄ heteroalkylene,
wherein at least one of X^{a}, R¹⁴, or L² is present.

In some embodiments, the linker is or comprises a cyclic group. In some embodiments, the linker has the structure of Formula IIb:

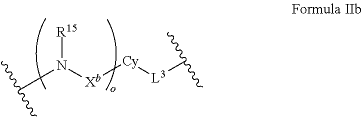

wherein o is 0 or 1;
X^{b} is C(O) or SO₂;
R¹⁵ is hydrogen or optionally substituted C₁-C₆ alkyl;
Cy is optionally substituted 3 to 8-membered cycloalkylene, optionally substituted 3 to 8-membered heterocycloalkylene, optionally substituted 6-10 membered arylene, or optionally substituted 5 to 10-membered heteroarylene; and
L³ is absent, —C(O)—, —SO₂—, optionally substituted C₁-C₄ alkylene or optionally substituted C₁-C₄ heteroalkylene.

In some embodiments, the linker is absent.

In some embodiments, W is hydrogen. In some embodiments, W is optionally substituted cyclopropyl, optionally substituted cyclobutyl, optionally substituted cyclopentyl, optionally substituted cyclohexyl, optionally substituted piperidine, optionally substituted piperazine, optionally substituted pyridine, or optionally substituted phenyl. In some embodiments, W is optionally substituted amino. In some embodiments, W is optionally substituted amido. In some embodiments, W is optionally substituted C₁-C₄ alkoxy. In some embodiments, W is optionally substituted C₁-C₄ alkyl. In some embodiments, W is optionally substituted C₁-C₄ hydroxyalkyl. In some embodiments, W is optionally substituted C₁-C₄ aminoalkyl. In some embodiments, W is optionally substituted C₁-C₄ haloalkyl. In some embodiments, W is optionally substituted C₁-C₄ guanidinoalkyl. In some embodiments, W is C₀-C₄ alkyl optionally substituted 3 to 11-membered heterocycloalkyl. In some embodiments, W is optionally substituted 3 to 10-membered cycloalkyl. In some embodiments, W is optionally substituted 3 to 10-membered heteroaryl. In some embodiments, W is optionally substituted 6- to 10-membered aryl.

In some embodiments, a strong hydrolyzer is contemplated, wherein the strong hydrolyzer is a compound comprising one of the following cores:

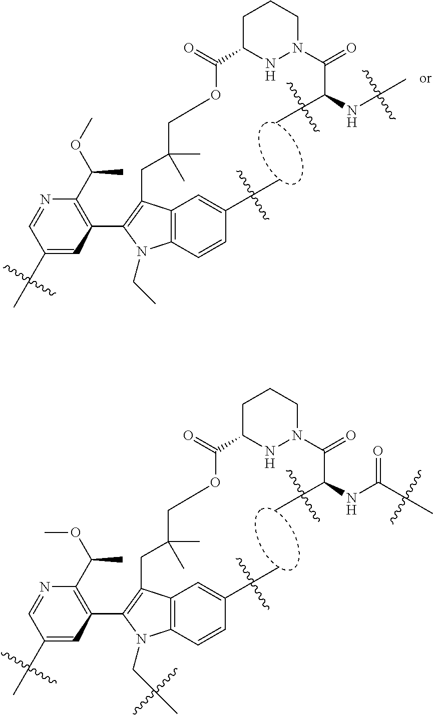

wherein

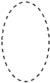

is one of the following:

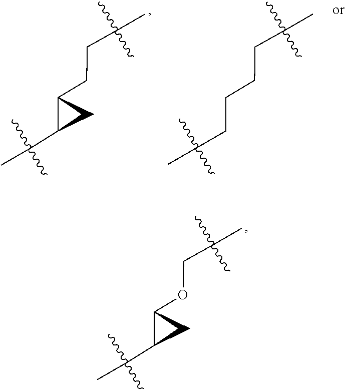 [SMILES: *CC[C@@H]1C[C@H]1* ; *CCCC* ; *CO[C@@H]1C[C@H]1*]

or a pharmaceutically acceptable salt thereof. In some embodiments,

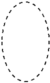

is one of the following:

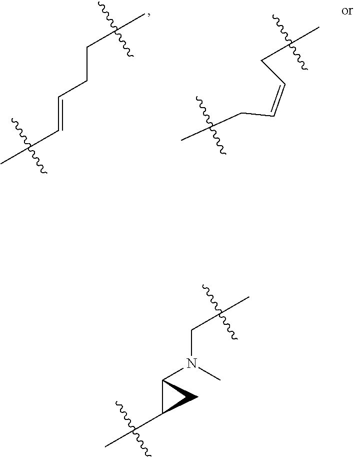

In some embodiments, a moderate hydrolyzer is contemplated, wherein the moderate hydrolyzer is a compound comprising one of the following cores:

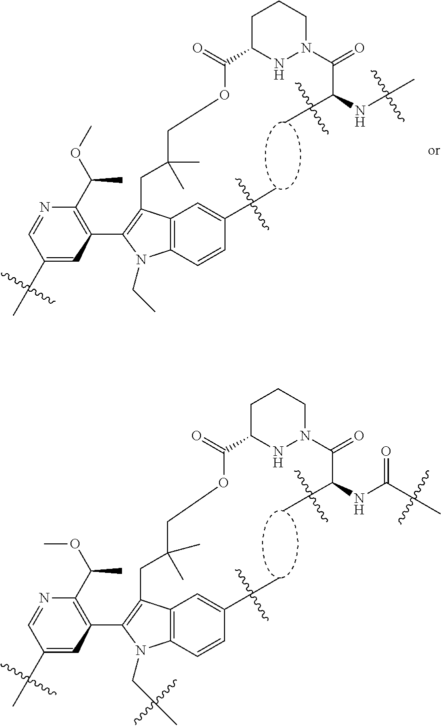

wherein

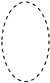

is

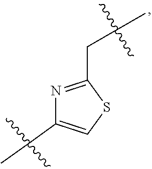 [SMILES: *Cc1nc(*)cs1]

or a pharmaceutically acceptable salt thereof. Examples of such compounds can be found in, e.g., WO 2021/091956, WO 2022/060836, and WO 2023/240263, each of which is incorporated herein by reference in its entirety.

In some embodiments, a weak hydrolyzer is contemplated, wherein the weak hydrolyzer is a compound comprising one of the following cores:

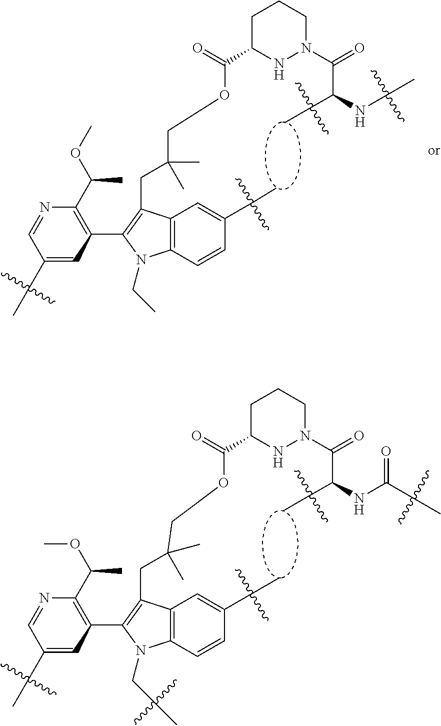

wherein

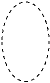

is one of the following:

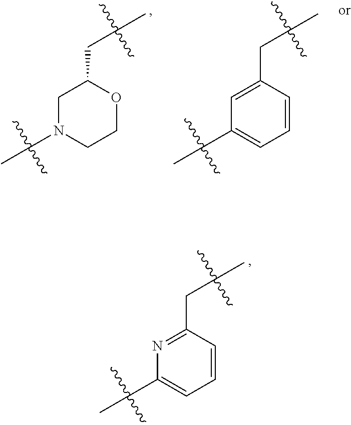 [SMILES: *C[C@H]1CN(*)CCO1 ; *Cc1cccc(*)c1 ; *Cc1cccc(*)n1]

or a pharmaceutically acceptable salt thereof.

Examples of such compounds can be found in, e.g., WO 2021/091956, WO 2022/060836 and WO 2023/240263, each of which is incorporated by reference in its entirety.

Also provided herein is a compound having the structure of Formula Ic, Id or Ie:

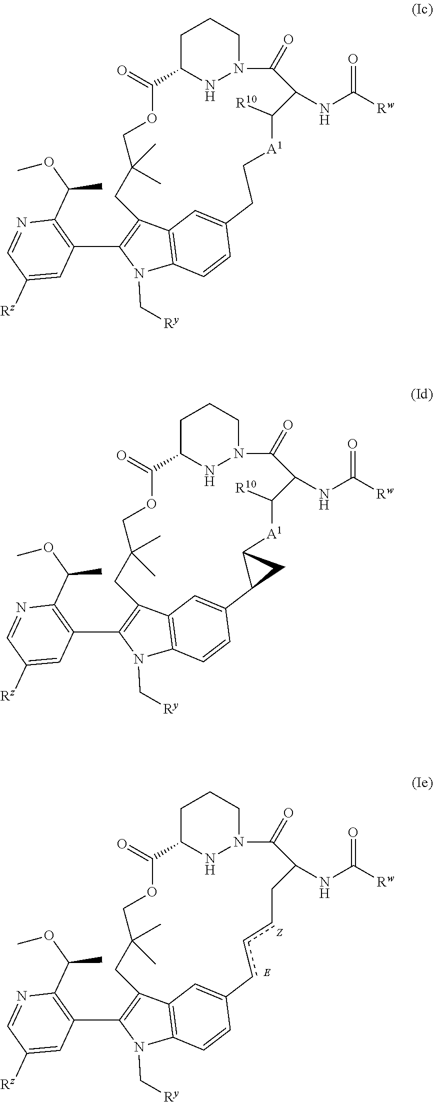

or a pharmaceutically acceptable salt thereof, wherein:
in Formula Ic:
   R^{w} is methylcyclopropyl;
   R^{y} is CH₃, CH₂F, CHF, or CHF₃;
   R^{z} is hydrogen or N-methylpiperazinyl;
   R¹⁰ is H; and
   A¹ is —CH₂—, —O—, or —NCH₃;
in Formula Id:
   R^{w} is methylcyclopropyl or dimethylcyclopropyl;
   R^{y} is CH₃;
   R^{z} is hydrogen or N-methylpiperazinyl;
   R¹⁰ is H; and
   A¹ is —CH₂, —O— or —NCH₃; or
in Formula Ie:
   either the E or Z double bond is present;
   R^{w} is methylcyclopropyl or dimethylcyclopropyl;
   R^{y} is CH₃;
   R^{z} is hydrogen or N-methylpiperazinyl;
   R¹⁰ is H; and
   A¹ is —CH₂—.

In some embodiments, a strong hydrolyzer is employed in a method disclosed herein. In some embodiments, a moderate hydrolyzer is not employed in a method of the present invention. In some embodiments, a weak hydrolyzer is not employed in a method of the present invention.

In some embodiments, a compound of the present disclosure is selected from Table 1, or a pharmaceutically acceptable salt or stereoisomer thereof. In some embodiments, a compound of the present disclosure is selected from Table 1, or a pharmaceutically acceptable salt or atropisomer thereof.

TABLE 1
Exemplary RAS(ON) GTP hydrolysis-promoting compounds that are strong hydrolyzers

| Ex. # | Structure |
|---|---|
| 1 (Compound A) | 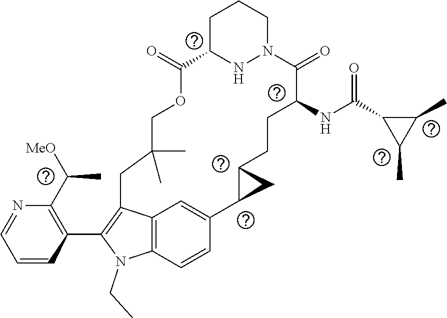 |
| 2 (Compound B) | 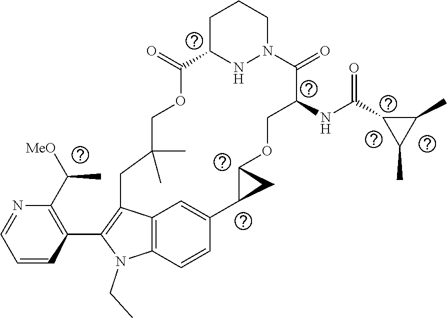 |
| 3 (Compound C) | 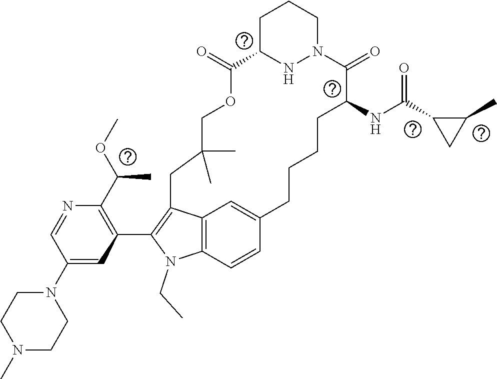 |
| 4 | 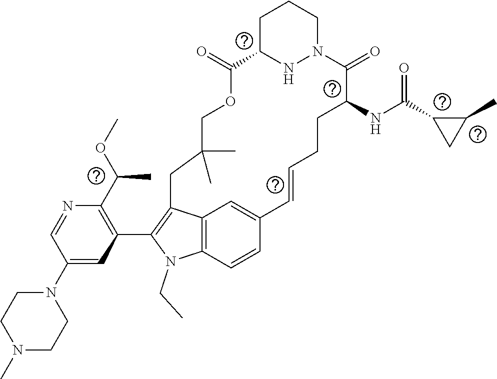 |
| 5 | 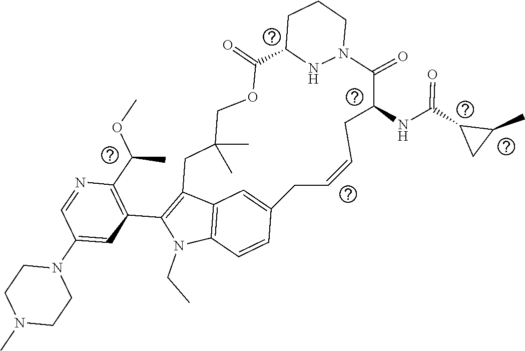 |
| 6 (Compound D) | 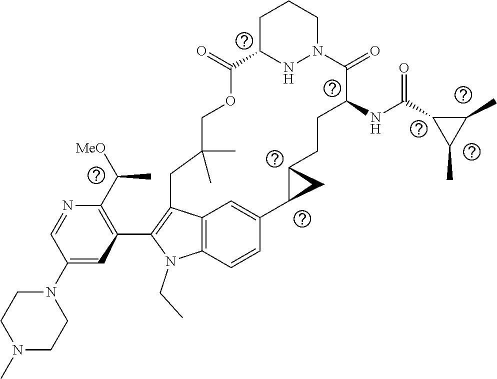 |
| 7 | 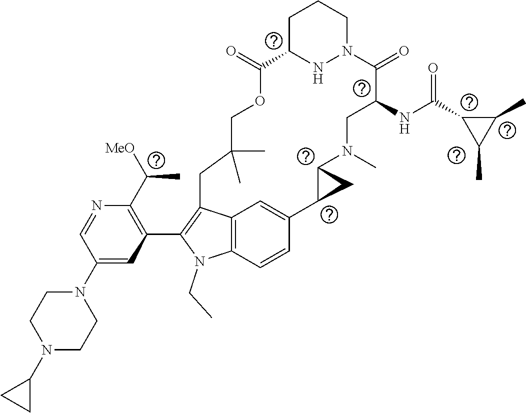 [symbol] |

[symbol]

indicates data missing or illegible when filed

In some embodiments, a RAS(ON) GTP hydrolysis-promoting compound of the present disclosure is selected from Table 2, or a pharmaceutically acceptable salt or stereoisomer thereof. In some embodiments, a compound of the present disclosure is selected from Table 2, or a pharmaceutically acceptable salt or atropisomer thereof.

TABLE 2
Exemplary RAS(ON) GTP hydrolysis-promoting compounds that are moderate
hydrolyzers or weak hydrolyzers

| Ex. # | Structure |
|---|---|
| 8 (Compound E) Moderate | 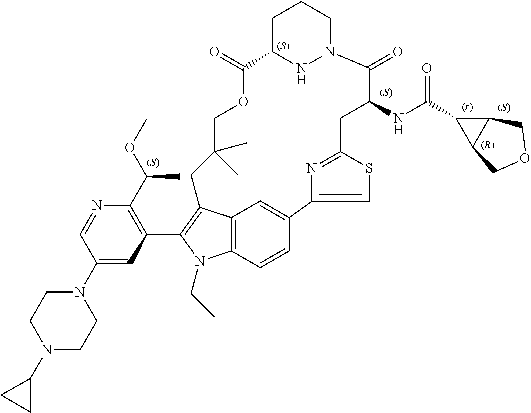 |
| 9 Weak | 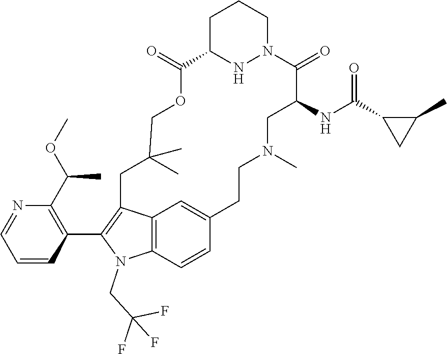 |
| 10 Moderate | 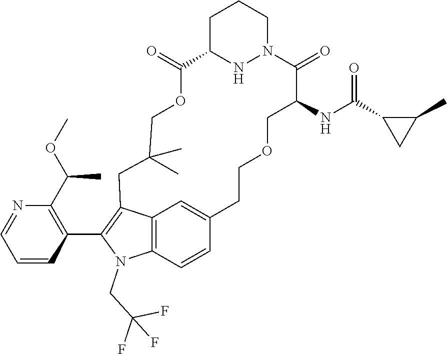 |
| 11 Moderate | 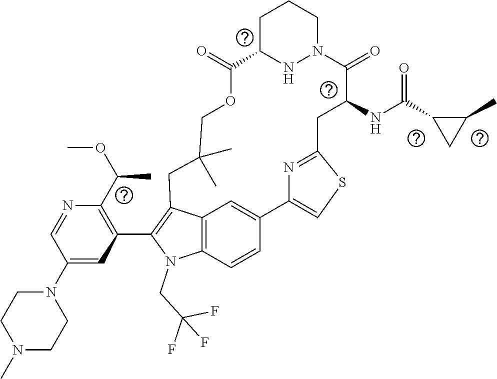 |
| 12 Moderate | 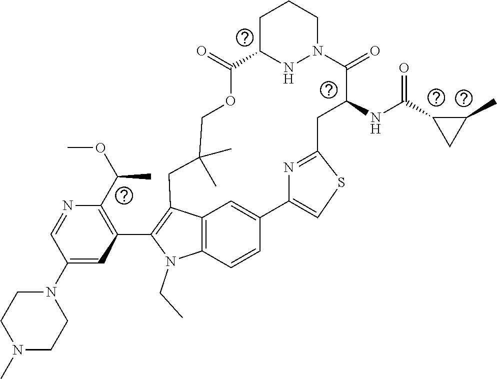 |
| 13 Moderate | 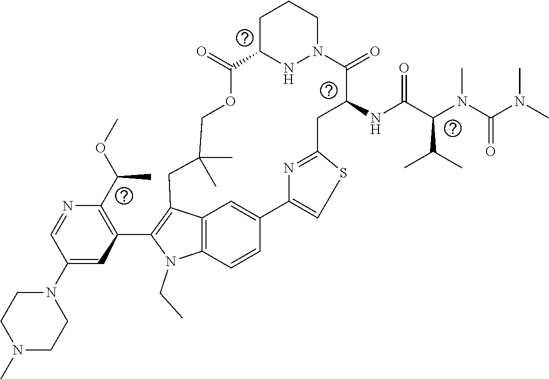 |
| 14 Moderate | 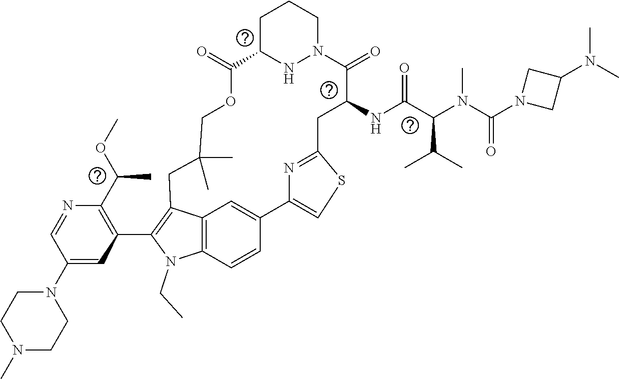 |
| 15 Moderate | 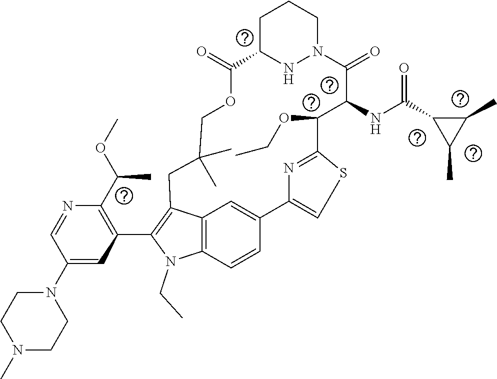 |
| 16 Moderate | 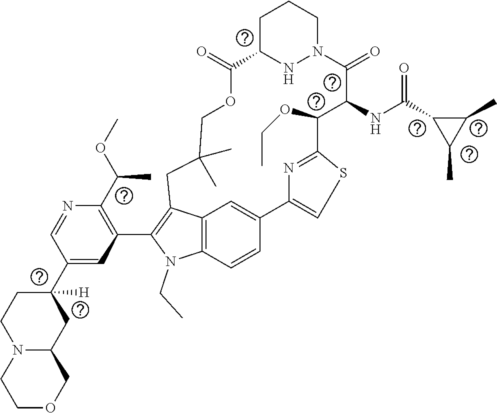 |
| 17 Weak | 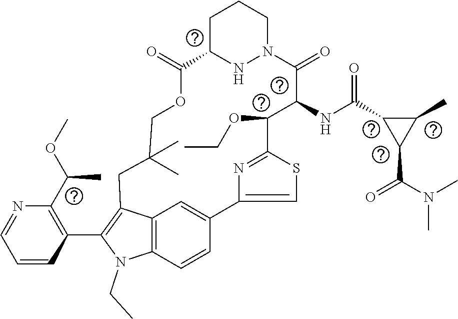 |
| 18 Weak | 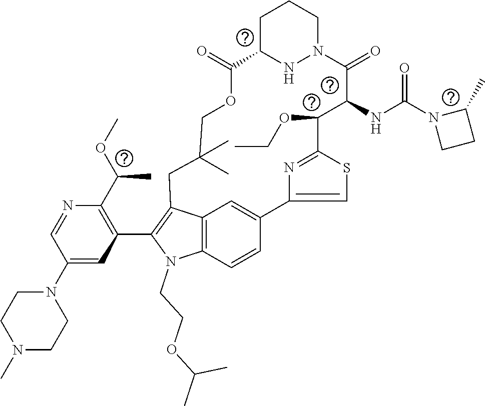 |
| 19 Moderate | 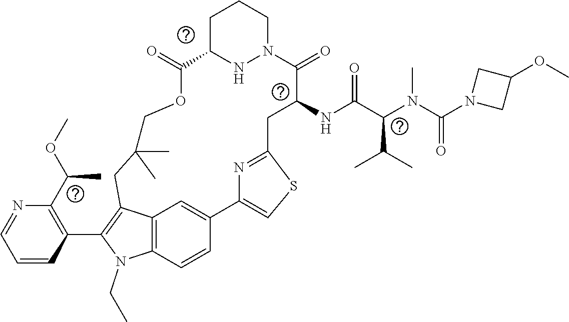 |
| 20 Weak | 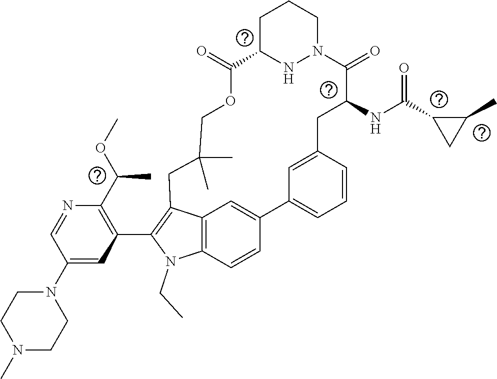 |
| 21 Weak | 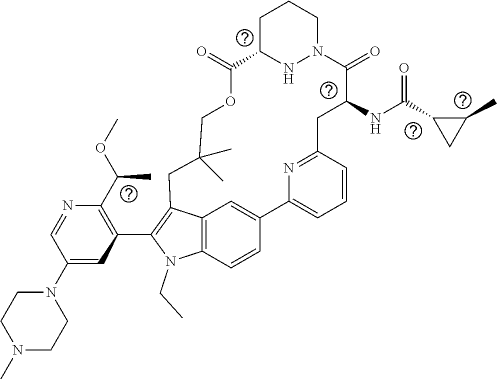 |
| 22 Weak | 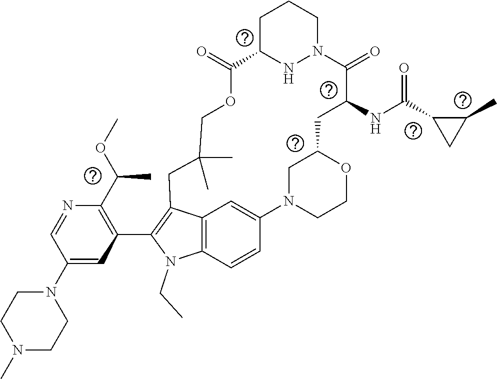 [symbol] |

[symbol]

indicates data missing or illegible when filed

The RAS(ON) GTP hydrolysis promoting compounds described herein may be made from commercially available starting materials or synthesized using known organic, inorganic, or enzymatic processes.

The compounds of the present invention can be prepared by methods known to those of skill in the art, such as those disclosed in WO 2021/091956, WO 2022/060836 and WO 2023/240263, in combination with known synthetic organic chemistry techniques, the disclosure of each of which is incorporated herein by reference. By way of example, compounds of the present invention can be synthesized using the methods described in the Schemes below, together with synthetic methods known in the art of synthetic organic chemistry, or variations thereon as appreciated by those skilled in the art. These methods include but are not limited to those methods described in the Schemes below.

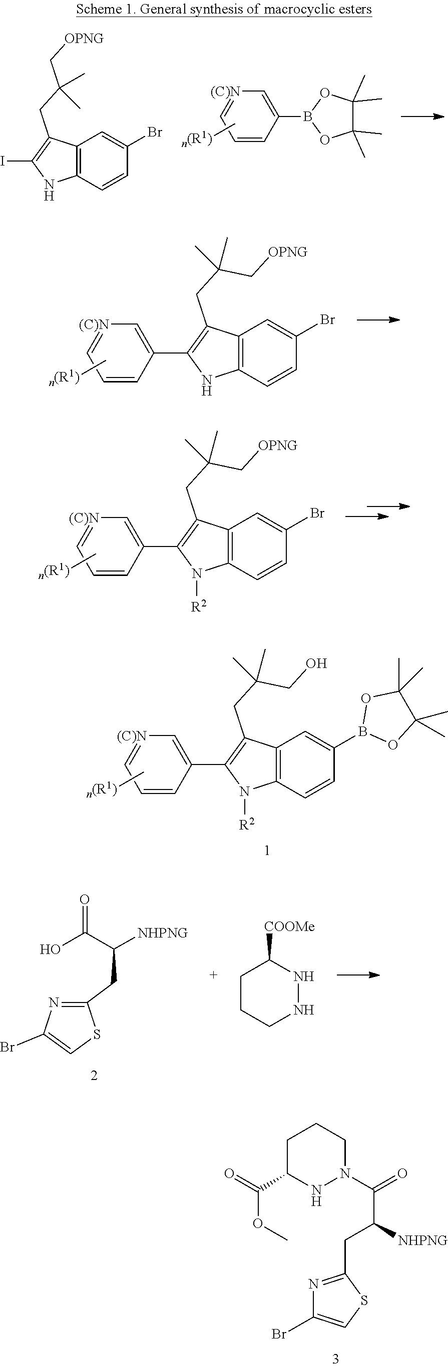

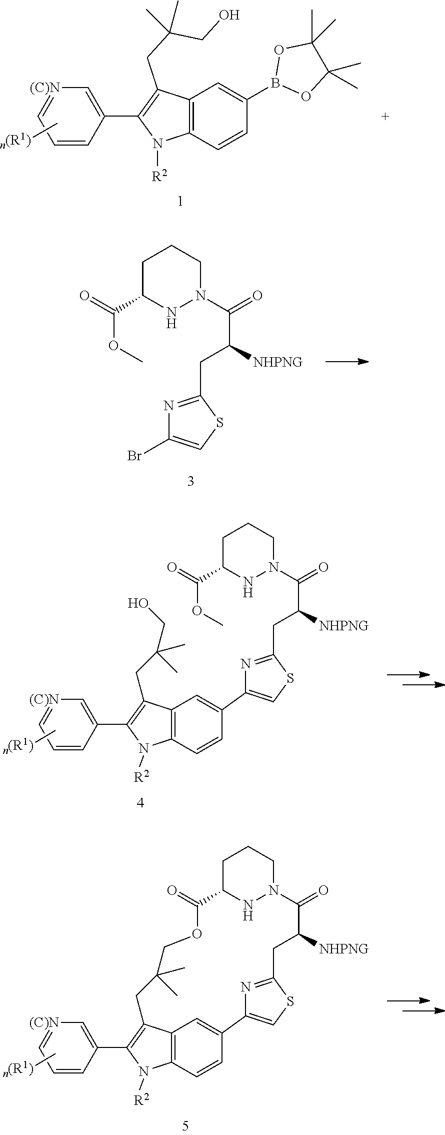

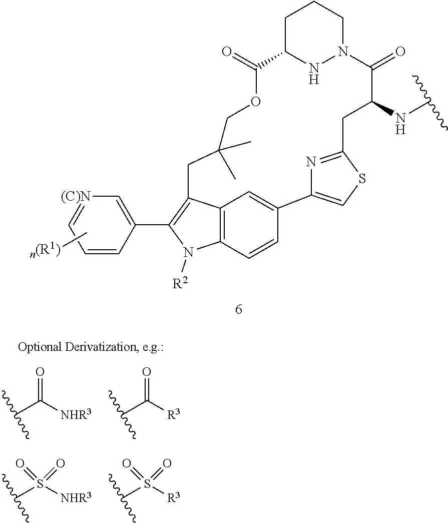

A general synthesis of macrocyclic esters is outlined in Scheme 1. An appropriately substituted indolyl boronic ester (1) can be prepared in four steps starting from protected 3-(5-bromo-2-iodo-1H-indol-3-yl)-2,2-dimethylpropan-1-ol and appropriately substituted boronic acid, including palladium mediated coupling, alkylation, de-protection, and palladium mediated borylation reactions.

Methyl-amino-3-(4-bromothiazol-2-yl)propanoyl)hexahydropyridazine-3-carboxylate (3) can be prepared via coupling of (S)-2-amino-3-(4-bromothiazol-2-yl)propanoic acid (2) with methyl (S)-hexahydropyridazine-3-carboxylate.

The final macrocyclic esters can be made by coupling of methyl-amino-3-(4-bromothiazol-2-yl)propanoyl)hexahydropyridazine-3-carboxylate (3) and an appropriately substituted indolyl boronic ester (1) in the presence of Pd catalyst followed by hydrolysis and macrolactonization steps to result in an appropriately protected macrocyclic intermediate (5). Deprotection and coupling with an appropriately substituted carboxylic acid (or other coupling partner) can result in a macrocyclic product. Additional deprotection or functionalization steps could be required to produce a final compound 6.

Further, with respect to Scheme 1, the thiazole may be replaced with an alternative optionally substituted 5 to 6-membered heteroarylene, or an optionally substituted 3 to 6-membered cycloalkylene, optionally substituted 3 to 6-membered heterocycloalkylene (e.g., morpholino), or optionally substituted 6-membered arylene (e.g., phenyl).

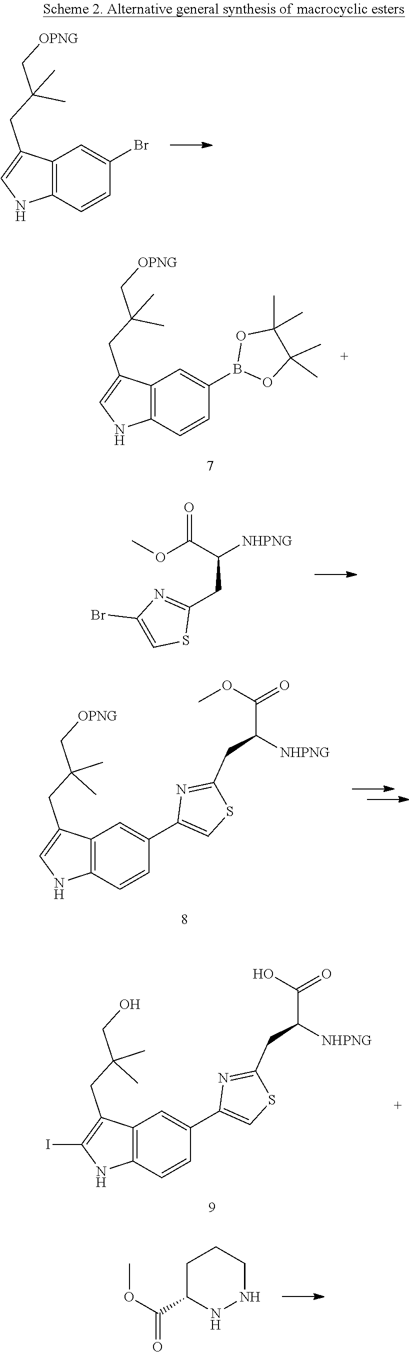

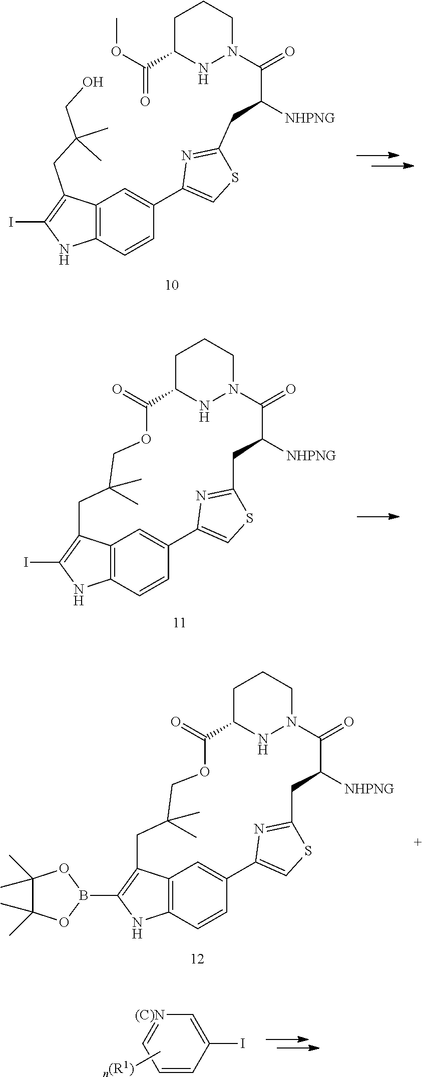

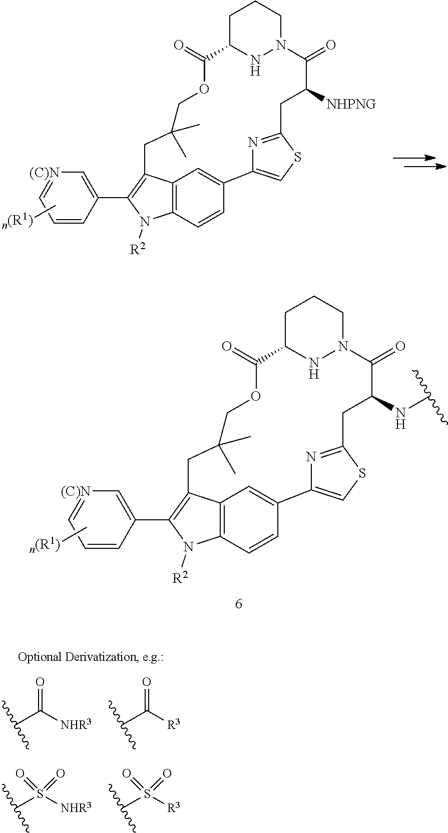

Alternatively, macrocyclic esters can be prepared as described in Scheme 2. An appropriately substituted and protected indolyl boronic ester (7) can be coupled in the presence of Pd catalyst with (S)-2-amino-3-(4-bromothiazol-2-yl)propanoic acid, followed by iodination, deprotection, and ester hydrolysis.

Subsequent coupling with methyl (S)-hexahydropyridazine-3-carboxylate, followed by hydrolysis and macrolactonization can result in iodo intermediate (11). Subsequent palladium mediated borylation and coupling in the presence of Pd catalyst with an appropriately substituted iodo aryl or iodo heteroaryl intermediate can yield an appropriately protected macrocyclic intermediate. Alkylation, deprotection and coupling with an appropriately substituted carboxylic acid carboxylic acid (or other coupling partner) results in a macrocyclic product. Additional deprotection or functionalization steps could be required to produce a final compound 6.

Further, with respect to Scheme 2, the thiazole may be replaced with an alternative optionally substituted 5 to 6-membered heteroarylene, or an optionally substituted 3 to 6-membered cycloalkylene, optionally substituted 3 to 6-membered heterocycloalkylene (e.g., morpholino), or optionally substituted 6-membered arylene (e.g., phenyl).

Compounds of Table 1 herein were prepared using methods disclosed herein or were prepared using methods described herein combined with the knowledge of one of skill in the art.

As described herein, the RAS(ON) GTP hydrolysis-promoting compound increases the rate of RAS GTP (guanosine triphosphate) hydrolysis relative to the rate of RAS GTP hydrolysis in the absence of the compound. GTP hydrolysis refers to the process by which a molecule GTP is cleaved into GDP (guanosine diphosphate) and inorganic phosphate (Pi) in the presence of water. In various embodiments, the rate of GTP hydrolysis is increased by about 5-100% (e.g., at least about or about 5%, 10%, 15%, 20%, 25%, 30%, 35%, 40%, 45%, 50%, 55%, 60%, 65%, 70%, 75%, 80%, 85%, 90%, or 95%, or about 100%, inclusive of all values and ranges between these values), 10-95%, 15-90%, 20-85%, 25-75%, 30-70%, 35-65%, 40-60%, 45-55%, or 50% compared to the rate of GTP hydrolysis in the absence of the compound. In various embodiments, the rate of GTP hydrolysis is increased by about 2-100 fold (e.g., at least about or about 2, 5, 10, 15, 20, 25, 30, 35, 40, 45, 50, 55, 60, 65, 70, 75, 80, 85, 90, 95, or 100 fold, inclusive of all values and ranges between these values) compared to the rate of GTP hydrolysis in the absence of the inhibitor.

Assays for measuring the rate of GTP hydrolysis are known in the art and contemplated herein. For example, the analysis of the rate of GTP hydrolysis can be performed in cell culture or in a cell-free system. Thin-layer chromatography (TLC) is a technique used to separate and visualize different molecules, including nucleotides such as GTP and GDP. In a typical GTP hydrolysis assay, the reaction mixture containing GTP, RAS and with or without a RAS(ON) GTP hydrolysis-promoting compound of the disclosure is incubated for a sufficient time and suitable conditions. The reaction is then stopped, and the reaction products (GDP and Pi) can then be separated by TLC and quantified. Colorimetric assays can be used which rely on the detection of a colored product generated by the reaction between Pi and a specific reagent, such as molybdate or malachite green. In this type of assay, the GTP hydrolysis reaction is carried out in the presence of the colorimetric reagent, and the absorbance of the colored product is measured over time. Fluorescence-based assays can also be used including fluorescent nucleotides, such as mant-GTP, which emit fluorescence upon hydrolysis. The reaction mixture containing the fluorescent nucleotide and RAS with or without a RAS(ON) GTP hydrolysis-promoting compound, and the decrease in fluorescence over time is monitored using a fluorescence spectrophotometer. Radioactive assays can also be used with radiolabeled GTP ([y-32P]GTP or [a-32P]GTP), which allows for highly sensitive detection of the reaction products (GDP and Pi) by scintillation counting. Additional methods are described herein, e.g., in the below examples.

In some embodiments, the hydrolysis is increased in the presence of a RAS(ON) GTP hydrolysis-promoting compound for a RAS protein that includes a G12C amino acid substitution relative to wild-type RAS or other RAS mutants. In some embodiments, GTP hydrolysis is increased for RAS that includes a G12D amino acid substitution relative to wild-type RAS or other KRAS mutants. In some embodiments, GTP hydrolysis is increased for RAS that includes a G12V amino acid substitution relative to wild-type RAS or other RAS mutants. In some embodiments, GTP hydrolysis is increased for RAS that includes a G13D amino acid substitution relative to wild-type RAS or other RAS mutants. In each of the above embodiments, RAS does not have a mutation at residue 61 relative to wild-type RAS.

### b) RAS(OFF) Inhibitors and RAS(OFF) Degraders

Compositions described herein may include one or more RAS(OFF) inhibitors. Numerous mutant-selective and pan-RAS inhibitors have been disclosed. A RAS(OFF) inhibitor may be administered or formulated in combination with a RAS(ON) GTP hydrolysis-promoting compound described herein. RAS(OFF) inhibitors are designed to inhibit RAS activity by targeting different regions of the RAS protein in its inactive state, preventing its activation and downstream signaling.

In some embodiments, a RAS(OFF) inhibitor is a KRAS(OFF) inhibitor that has a molecular weight of under 700 Da. The term “KRAS(OFF) inhibitor” refers to any RAS(OFF) inhibitor that binds to KRAS in its GDP-bound “OFF” position. In some embodiments, the KRAS(OFF) inhibitor is specific for a KRAS^{G12C} mutation. KRAS^{G12C}(OFF) inhibitors use a covalent binding group that allows them to selectively target the KRAS^{G12C} mutant protein, and many such inhibitors comprise a pyrimidine core. KRASG12C(OFF) inhibitors all target the same cysteine residue in the KRAS^{G12C} mutant protein, leading to a conformational change that locks the protein in an inactive state. KRAS^{G12C}(OFF) inhibitors includes, for example, AMG510 (sotorasib), MRTX849 (adagrasib), MRTX1257, GDC-6036 (divarasib), JDQ443 (opnurasib), ERAS-3490, LY3537982 (olomorasib), BI 1823911, BPI-421286, JAB-3312, JAB-21000, JAB-21822 (glecirasib), D-1553, D3S-001, HBI-2438, HS-10370, MK-1084, YL-15293, BBO—8520 (ON/OFF inhibitor), FMC-376 (ON/OFF inhibitor), GEC255, and GFH925 (1B1351). In some embodiments, the KRAS(OFF) inhibitor is selected from AMG 510 and MRTX849. In some embodiments, the KRAS(OFF) inhibitor is AMG 510. In some embodiments, the KRAS(OFF) inhibitor is selected from BPI-421286, JNJ-74699157 (ARS-3248), LY3537982, MRTX1257, ARS853, ARS1620, and GDC-6036.

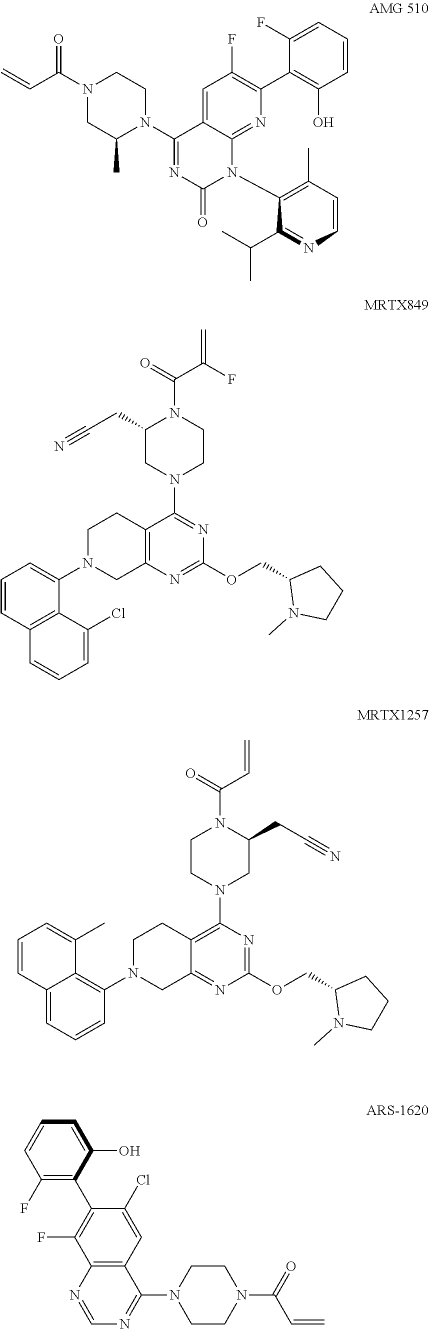

In some embodiments, a KRAS(OFF) inhibitor is specific for a KRAS^{G12D} mutation. Many KRAS^{G12D}(OFF) inhibitors have been developed using RAS(OFF) G12C inhibitors as a starting point, thus share the backbone of G12C inhibitors in combination with other chemical moieties such as piperazine-based compounds. Non-limiting examples of KRAS^{G12D}(OFF) inhibitors include MRTX1133, MRTX282, JAB-22000, ERAS-4, ERAS-5024, HRS-4642, BI-2852, ASP3082, TH-Z827, TH-Z835, QTX-3046, FGH375 (VS-7375), INCB161734 and KD-8. In some embodiments, the KRAS(OFF) inhibitor is MRTX1133.

Reference to “MRTX1133”, “TH-Z827”, “TH-Z835”, and “KD-8” herein means the following compounds:

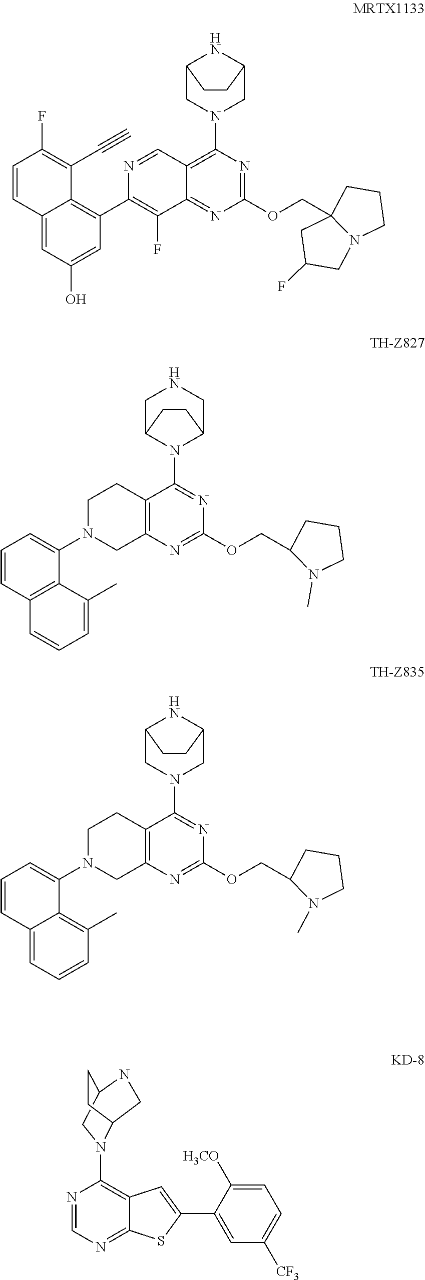 [SMILES: C#Cc1c(F)ccc2cc(O)cc(-c3ncc4c(N5CC6CCC(C5)N6)nc(OCC56CCCN5CC(F)C6)nc4c3F)c12 ; Cc1cccc2cccc(N3CCc4c(nc(OCC5CCCN5C)nc4N4C5CCC4CNC5)C3)c12 ; Cc1cccc2cccc(N3CCc4c(nc(OCC5CCCN5C)nc4N4CC5CCC(C4)N5)C3)c12 ; COc1ccc(C(F)(F)F)cc1-c1cc2c(N3CC4CCC3CN4)ncnc2s1]

In some embodiments, the small molecule RAS(OFF) inhibitor is specific for a KRAS^{G12V} mutation. In some embodiments, the small molecule RAS(OFF) inhibitor is specific for a KRAS^{G13D} mutation. In some embodiments, the small molecule RAS(OFF) inhibitor is specific for a pan-RAS(OFF) inhibitor. In some embodiments, reference to the term RAS(OFF) inhibitor includes any such RAS(OFF) inhibitor disclosed in any one of the following patent applications: WO 2024056063, WO 2024055112, WO 2024054926, WO 2024054647, WO 2024054625, WO 2024051763, WO 2024051721, WO 2024050742, WO 2024050640, WO 2024046406, WO 2024046370, WO 2024045066, WO 2024044667, WO 2024044649, WO 2024044334, WO 2024041621, WO 2024041606, WO 2024041589, WO 2024041573, WO 2024040131, WO 2024040109, WO 2024040080, WO 2024036270, WO 2024034657, WO 2024034593, WO 2024034591, WO 2024034123, WO 2024032747, WO 2024032704, WO 2024032703, WO 2024032702, WO 2024031088, WO 2024030647, WO 2024030633, WO 2024029613, WO 2024022507, WO 2024022444, WO 2024020159, WO 2024019103, WO 2024017859, WO 2024017392, WO 2024015731, WO 2024015262, WO 2024012456, WO 2024009191, WO 2024008179, WO 2024008178, WO 2024008068, WO 2024006445, WO 2024006424, WO 2024002373, WO 2023287896, WO 2023287730, WO 2023284881, WO 2023284730, WO 2023284537, WO 2023283933, WO 2023283213, WO 2023280280, WO 2023280136, WO 2023280026, WO 2023278600, WO 2023274383, WO 2023327324, WO 2023246914, WO 2023246903, WO 2023246777, WO 2023244713, WO 2023244615, WO 2023244604, WO 2023244600, WO 2023244599, WO 2023230190, WO 2023226630, WO 2023225302, WO 2023225252, WO 2023220421, WO 2023219941, WO 2023217148, WO 2023215802, WO 2023215801, WO 2023213269, WO 2023212548, WO 2023208005, WO 2023205719, WO 2023199180, WO 2023198191, WO 2023197984, WO 2023190748, WO 2023185864, WO 2023183755, WO 2023183585, WO 2023179703, WO 2023179629, WO 2023173017, WO 2023173016, WO 2023173014, WO 2023172737, WO 2023171781, WO 2023159087, WO 2023159086, WO 2023154766, WO 2023152255, WO 2023151674, WO 2023151621, WO 2023150394, WO 2023150284, WO 2023143623, WO 2023143605, WO 2023143352, WO 2023143352, WO 2023143312, WO 2023141570, WO 2023141300, WO 2023138662, WO 2023138601, WO 2023138589, WO 2023138524, WO 2023133183, WO 2023133181, WO 2023130012, WO 2023125989, WO 2023125627, WO 2023122662, WO 2023122154, WO 2023120742, WO 2023119677, WO 2023117681, WO 2023116934, WO 2023116895, WO 2023114733, WO 2023105491, WO 2023104018, WO 2023103906, WO 2023103523, WO 2023101928, WO 2023099624, WO 2023099624, WO 2023099620, WO 2023099612, WO 2023099608, WO 2023099592, WO 2023098832, WO 2023098425, WO 2023097227, WO 2023081840, WO 2023081476, WO 2023078424, WO 2023077441, WO 2023072297, WO 2023072188, WO 2023066371, WO 2023064857, WO 2023061463, WO 2023061294, WO 2023057985, WO 2023056951, WO 2023056421, WO 2023051586, WO 2023049697, WO 2023046135, WO 2023045960, WO 2023041059, WO 2023041059, WO 2023040989, WO 2023040513, WO 2023039240, WO 2023039020, WO 2023036282, WO 2023034290, WO 2023030517, WO 2023030495, WO 2023030385, WO 2023025116, WO 2023020523, WO 2023020521, WO 2023020519, WO 2023020518, WO 2023020347, WO 2023018812, WO 2023018810, WO 2023018809, WO 2023018699, WO 2023014979, WO 2023014006, WO 2023004102, WO 2023003417, WO 2023001141, WO 2023001123, WO 2022271658, WO 2022269508, WO 2022266167, WO 2022266069, WO 2022266015, WO 2022265974, WO 2022261154, WO 2022261154, WO 2022251576, WO 2022251296, WO 2022237815, WO 2022232332, WO 2022232331, WO 2022232320, WO 2022232318, WO 2022223037, WO 2022221739, WO 2022221528, WO 2022221386, WO 2022216762 (e.g., Compound 44 or Compound 66a), WO 2022192794, WO 2022192790, WO 2022188729, WO 2022187411, WO 2022184178, WO 2022173870, WO 2022173678, WO 2022135346, WO 2022133731, WO 2022133038, WO 2022133345, WO 2022132200, WO 2022119748, WO 2022109485, WO 2022109487, WO 2022066805, WO 2022002102, WO 2022002018, WO 2021259331, WO 2021257828, WO 2021252339, WO 2021248095, WO 2021248090, WO 2021248083, WO 2021248082, WO 2021248079, WO 2021248055, WO 2021245051, WO 2021244603, WO 2021239058, WO 2021231526, WO 2021228161, WO 2021219090, WO 2021219090, WO 2021219072, WO 2021218939, WO 2021217019, WO 2021216770, WO 2021215545, WO 2021215544, WO 2021211864, WO 2021190467, WO 2021185233, WO 2021180181, WO 2021175199, 2021173923, WO 2021169990, WO 2021169963, WO 2021168193, WO 2021158071, WO 2021155716, WO 2021152149, WO 2021150613, WO 2021147967, WO 2021147965, WO 2021143693, WO 2021142252, WO 2021141628, WO 2021139748, WO 2021139678, WO 2021129824, WO 2021129820, WO 2021127404, WO 2021126816, WO 2021126799, WO 2021124222, WO 2021121371, WO 2021121367, WO 2021121330, WO 2021113595, WO 2021107160, WO 2021106231, WO 2021088458, WO 2021086833, WO 2021085653, WO 2021081212, WO 2021058018, WO 2021057832, WO 2021055728, WO 2021031952, WO 2021027911, WO 2021023247, WO 2020259513, WO 2020259432, WO 2020234103, WO 2020233592, WO 2020216190, WO 2020178282, WO 2020146613, WO 2020118066, WO 2020113071, WO 2020106647, WO 2020102730, WO 2020101736, WO 2020097537, WO 2020086739, WO 2020081282, WO 2020050890, WO 2020047192, WO 2020035031, WO 2020028706, WO 2019241157, WO 2019232419, WO 2019217691, WO 2019217307, WO 2019215203, WO 2019213526, WO 2019213516, WO 2019155399, WO 2019150305, WO 2019110751, WO 2019099524, WO 2019051291, WO 2018218070, WO 2018218071, WO 2018218069, WO 2018217651, WO 2018206539, WO 2018143315, WO 2018140600, WO 2018140599, WO 2018140598, WO 2018140514, WO 2018140513, WO 2018140512, WO 2018119183, WO 2018112420, WO 2018068017, WO 2018064510, WO 2017201161, WO 2017172979, WO 2017100546, WO 2017087528, WO 2017058807, WO 2017058805, WO 2017058728, WO 2017058902, WO 2017058792, WO 2017058768, WO 2017058915, WO 2017015562, WO 2016168540, WO 2016164675, WO 2016049568, WO 2016049524, WO 2015054572, WO 2014152588, WO 2014143659, WO 2013155223, CN 117683051, CN 117645627, CN 117624194, CN 117624190, CN 117586280, CN 117486901, CN 117466917, CN 117462688, CN 117362315, CN 117327102, CN 117327094, CN 117327074, CN 117285590, CN 117263959, CN 117247382, CN 117186095, CN 117164605, CN 116969977, CN 116925075, CN 116891489, CN 116731045, CN 116731044, CN 116554208, CN 116514846, CN 116478184, CN 116478141, CN 116410145, CN 116375742, CN 116354988, CN 116332948, CN 116332938, CN 116327956, CN 116262759, CN 116217592, CN 116199703, CN 116162099, CN 116143806, CN 116143805, CN 116120315, CN 116102559, CN 115960105, CN 115894520, CN 115872979, CN 115850267, CN 115785199, CN 115785124, CN 115785124, CN 115724842, CN 115716840, CN 115703775, CN 115611923, CN 115611898, CN 115583937, CN 115572278, CN 115557949, CN 115521312, CN 115504976, CN 115490709, CN 115466272, CN 115433183, CN 115433179, CN 115403575, CN 115385938, CN 115385937, CN 115385912, CN 115381786, CN 115368383, CN 115368382, CN 115368381, CN 115353506, CN 115322158, CN 115304623, CN 115304602, CN 115197245, CN 115181106, CN 114989195, CN 114989166, CN 114989147, CN 114920741, CN 114920739, CN 114907387, CN 114874234, CN 114874201, CN 114716436, CN 114716435, CN 114685532, CN 114685460, CN 114591319, CN 114539293, CN 114539286, CN 114539246, CN 114437107, CN 114437084, CN 114409653, CN 114380827, CN 114195804, CN 114195788, CN 114057776, CN 114057744, CN 114057743, CN 113999226, CN 113980032, CN 113980014, CN 113929676, CN 113754653, CN 113683616, CN 113563323, CN 113527299, CN 113527294, CN 113527293, CN 113493440, CN 113429405, CN 113248521, CN 113087700, CN 113024544, CN 113004269, CN 112920183, CN 112778284, CN 112390818, CN 112390788, CN 112300196, CN 112300194, CN 112300173, CN 112225734, CN 112142735, CN 112110918, CN 112094269, CN 112047937, and CN 109574871, each of which is incorporated herein by reference in its entirety, including the compound structures disclosed therein.

In some embodiments, the RAS(OFF) inhibitor is a peptide-based inhibitor. Peptide-based RAS(OFF) inhibitors have been developed that target specific regions of the RAS protein, such as the Switch II region or the RAS-effector interface. Non-limiting examples include the K-Ras-binding peptide (KRpep-2d), the Ras inhibitory peptide (Rasln) and LUNA18 (NCT05012618). Peptide-based RAS(OFF) inhibitors are a class of compounds that target the RAS protein by disrupting its interaction with its downstream effectors or other signaling proteins. These inhibitors are typically designed to mimic the binding motifs of RAS-interacting proteins or other RAS effectors, such as RAF or PI3K. By binding to RAS at the same site as these effectors, peptide-based inhibitors can effectively compete with these proteins and prevent the activation of downstream signaling pathways.

Peptide-based RAS(OFF) inhibitors can be further classified into two main categories: those that target the RAS-effector interface, and those that target other regions of the RAS protein. Peptide-based inhibitors that target the RAS-effector interface are designed to bind to the switch regions of RAS that are critical for its interaction with downstream effectors, such as RAF or PI3K. These inhibitors typically contain amino acid residues that are similar to those found in the binding motifs of RAS-interacting proteins or effectors and are often designed to form hydrogen bonds or other interactions with key residues on the surface of RAS.

Peptide-based RAS(OFF) inhibitors that target other regions of the RAS protein are typically designed to disrupt other interactions that are critical for the activation or signaling of RAS. For example, some peptide-based inhibitors are designed to bind to the hypervariable region of RAS, which is thought to play a role in membrane localization and anchoring of the protein. By binding to this region, peptide-based inhibitors can prevent the proper localization of RAS to the plasma membrane, which is necessary for its activation and signaling.

Several common motifs have been identified as important for the binding of RAS-interacting proteins and effectors and are often used in the design of peptide-based inhibitors. One example is the RAF-binding domain (RBD), which is found in many RAS-interacting proteins and is important for the interaction of RAS with downstream effectors such as RAF. The RBD contains a conserved amino acid sequence (Arg-Xaa-Arg) that is critical for binding to RAS, and this motif has been incorporated into several peptide-based inhibitors designed to disrupt the RAS-RAF interaction. Another example is the RAS-binding domain (RBD) of PI3K, which is important for the interaction of RAS with this downstream effector. The RBD of PI3K contains several conserved amino acid residues (such as Arg-Arg-Trp) that are critical for binding to RAS, and these motifs have been used in the design of peptide-based inhibitors that target the RAS-PI3K interaction. Other common motifs used in peptide-based RAS(OFF) inhibitors include the Ras-binding domain (RBD) of other RAS-interacting proteins such as RaIGDS and SOS, as well as sequences that mimic the structure of the switch regions of RAS itself. These motifs are typically used to optimize the binding affinity and selectivity of the inhibitor for the desired target protein or interaction.

In some embodiments, the RAS(OFF) inhibitor is an antibody or antigenic binding peptide specific for RAS(OFF). Antibodies have been developed that bind to specific regions of the RAS protein, such as the Switch II region or the RAS-effector interface. For example, some antibodies have been developed that target the switch regions of RAS proteins, which are critical for the activation of these proteins and their interaction with downstream effectors. Binding of these antibodies to the switch regions can prevent the conformational changes required for RAS activation and downstream signaling. Another approach involves the use of antibodies that target RAS-interacting proteins or downstream effectors, such as RAF or PI3K. Binding of these antibodies to their target proteins can disrupt the RAS-dependent signaling pathways and inhibit the growth and survival of cancer cells. Additionally, some antibodies have been developed that can induce the internalization and degradation of RAS proteins, leading to their depletion and inhibition of downstream signaling. For example, some antibodies have been developed that recognize the unique structure of mutant RAS proteins and target them for degradation via the ubiquitin-proteasome pathway. Non-limiting examples of KRAS(OFF)-specific inhibitory antibodies include anti-p21ser, and K27 (DARPin) (see, e.g., Khan et al, Biochim Biophys Acta Mol Cell Res. 2020 February; 1867(2):118570).

In any embodiment employing a RAS(OFF) inhibitor herein, a RAS(OFF) degrader targeting the OFF state of RAS may alternatively be employed. These degraders are known in the art. RAS degraders may be found, for example, in one or more of the following applications: WO 2024055112, WO 2024054625, WO 2024050742, WO 2024044334, WO 2024040080, WO 2024034657, WO 2024034593, WO 2024034591, WO 2024034123, WO 2024029613, WO 2024020159, WO 2024019103, WO 2024017392, WO 2023185864, WO 2023171781, WO 2023141570, WO 2023138524, WO 2023130012, WO 2023116934, WO 2023099620, WO 2023081476, WO 2023077441, and CN 115785199, each of which is incorporated herein by reference in its entirety, including the compound structures disclosed therein which are specifically incorporated herein by reference. The RAS(OFF) inhibitor structures disclosed herein provide a means for inhibiting RAS(OFF).

### c) RTK Inhibitors

Compositions and methods described herein may include a RAS(ON) GTP hydrolysis-promoting compound in combination with one or more receptor tyrosine kinase inhibitors. A receptor tyrosine kinase (RTK) inhibitor is a type of molecule (e.g., small molecule, antibody, and nucleic acid) that binds to and blocks the activity of receptor tyrosine kinases or their ligands. RTKs are proteins found on the surface of cells that play a critical role in cell signaling and growth and have been developed as therapeutics for a range of diseases, including cancer, diabetes, and autoimmune disorders.

### i) EGFR Inhibitors

In some embodiments, compositions and methods described herein may include one or more EGFR inhibitors. An EGFR inhibitor may be administered or formulated in combination with a RAS(ON) GTP hydrolysis-promoting compound and/or any additional therapeutic agent described herein. EGFR inhibitors include, but are not limited to, small molecule antagonists, antibody inhibitors, or specific antisense nucleotide or siRNA. Useful antibody inhibitors of EGFR include cetuximab (Erbitux®), panitumumab (Vectibix®), zalutumumab, nimotuzumab, and matuzumab. Further antibody-based EGFR inhibitors include any anti-EGFR antibody or antibody fragment that can partially or completely block EGFR activation by its natural ligand. Non-limiting examples of antibody-based EGFR inhibitors include those described in Modjtahedi et al., Br. J. Cancer 1993, 67:247-253; Teramoto et al., Cancer 1996, 77:639-645; Goldstein et al., Clin. Cancer Res. 1995, 1:1311-1318; Huang et al., 1999, Cancer Res. 15:59(8):1935-40; and Yang et al., Cancer Res. 1999, 59:1236-1243. The EGFR inhibitor can be monoclonal antibody Mab E7.6.3 (Yang, 1999 supra), or Mab C225 (ATCC Accession No. HB-8508), or an antibody or antibody fragment having the binding specificity thereof.

Small molecule antagonists of EGFR include gefitinib (Iressa®), Lazertinib, erlotinib (Tarceva®), and lapatinib (TykerB®). See, e.g., Yan et al., Pharmacogenetics and Pharmacogenomics In Oncology Therapeutic Antibody Development, BioTechniques 2005, 39(4):565-8; and Paez et al., EGFR Mutations In Lung Cancer Correlation With Clinical Response To Gefitinib Therapy, Science 2004, 304(5676):1497-500. In some embodiments, the EGFR inhibitor is osimertinib (Tagrisso®). In some embodiments, an EGFR inhibitor is one or more of cetuximab, gefitinib (Iressa), erlotinib (Tarceva), and afatinib (Gilotrif. Additional non-limiting examples of small molecule EGFR inhibitors include any of the EGFR inhibitors described in Traxler et al., Exp. Opin. Ther. Patents 1998, 8(12):1599-1625. An EGFR inhibitor may be ERAS-801. In some embodiments, an EGFR inhibitor is an ERBB inhibitor. In humans, the ERBB family contains HER1 (EGFR, ERBB1), HER2 (NEU, ERBB2), HER3 (ERBB3), and HER (ERBB4). In some embodiments, reference to the term EGFR inhibitor includes any such EGFR inhibitor disclosed in any one of the following patent applications: WO 2023041071, WO 2023049312, WO 2023020600, WO 2023284747, WO 2022206797, WO 2022258977, WO 2022033416, WO 2022033410, WO 2022105908, WO 2022100641, WO 2022014639, WO 2022007841, WO 2021018009, WO 2021057882, WO 2021252661, WO 2021018003, WO 2021073498, WO 2021238827, WO 2020254547, WO 2020216371, WO 2020147838, WO 2020207483, WO 2020254572, WO 2020001350, WO 2021001351, WO 2019164948, WO 2019218958, WO 2019046775, WO 2019015655, WO 2018121758, WO 2018218963, WO 2017220007, WO 2017205459, WO 2017161937, WO 2016192609, WO 199633980, WO 199630347, WO 199730034, WO 199730044, WO 199738994, WO 199749688, WO 199802434, WO 199738983, WO 199519774, WO 199519970, WO 199713771, WO 199802437, WO 199802438, WO 199732881, WO 199833798, WO 199732880, WO 199732880, WO 199702266, WO 199727199, WO 199807726, WO 1997/34895, WO 199631510, WO 199814449, WO 199814450, WO 199814451, WO 199509847, WO 199719065, WO 199817662, WO 199935146, WO 199935132, WO 199907701, WO 199220642, DE 19629652, EP 682027, EP 837063, EP 0787772, EP 0520722, EP 0566226, CN 115960018, CN 110283162, CN 114044774, CN111973601, CN 111973602, and CN113896744, each of which is incorporated herein by reference in its entirety, including the compound structures disclosed therein which are specifically incorporated herein by reference.

### ii) HER2 Inhibitors

In some embodiments, compositions and methods described herein may include one or more HER2 inhibitors. A HER2 inhibitor may be administered or formulated in combination with a RAS(ON) GTP hydrolysis-promoting compound and/or any additional therapeutic agent described herein. In some embodiments, an HER2 inhibitor is one or more of tucatinib, rastuzumab (Herceptin), pertuzumab (Perjeta), lapatinib (Tykerb), ado-trastuzumab emtansine (Kadcyla), and neratinib (Nerlynx). Non-limiting examples of HER2 inhibitors include monoclonal antibodies such as trastuzumab (Herceptin®) and pertuzumab (Perjeta®); small molecule tyrosine kinase inhibitors such as gefitinib (Iressa®), erlotinib (Tarceva®), pilitinib, CP-654577, CP-724714, canertinib (CI 1033), HKI-272, lapatinib (GW-572016; Tykerb®), PKI-166, AEE788, BMS-599626, HKI-357, BIBW 2992, ARRY-334543, and JNJ-26483327. In some embodiments, reference to the term HER2 inhibitor includes any such HER2 inhibitor disclosed in any one of the following patent applications: WO 2021156178, WO 2021156180, WO 2021213800, WO 2021088987, WO 2013561183, and WO 2013056108, each of which is incorporated herein by reference in its entirety, including the compound structures disclosed therein which are specifically incorporated herein by reference.

iii) MET Inhibitors

In some embodiments, compositions and methods described herein may include one or more MET inhibitors. A MET inhibitor may be administered or formulated in combination with a RAS(ON) GTP hydrolysis-promoting compound and/or any additional therapeutic agent described herein. In some embodiments, a MET inhibitor is one or more of Crizotinib (Xalkori), Cabozantinib (Cometriq, Cabometyx), Capmatinib (Tabrecta), Tepotinib (Tepmetko), Savolitinib (Volitinib), Onartuzumab (MetMab), Foretinib (GSK1363089), MGCD-265 (Amuvatinib), SU11274, and SU5416. In some embodiments, reference to the term MET inhibitor includes any such MET inhibitor disclosed in any one of the following patent applications: WO 2022226168, WO 2021222045, WO 2020047184, WO 2020015744, WO 2020244654, WO 2020156453, WO 2019206268, WO 2018077227, WO 2017012539, WO 2016015653, WO 2016012963, WO 2012015677, WO 2011162835, WO 2010089507, WO 2009091374, WO 2009056692, WO 2008051547, WO 2007130468, US 2012237524, CN 103497177, CN 107311983, CN 107382968, CN 110218191, and TW201331206, each of which is incorporated herein by reference in its entirety, including the compound structures disclosed therein which are specifically incorporated herein by reference.

### iv) AXL Inhibitors

In some embodiments, compositions and methods described herein may include one or more AXL inhibitors. An AXL inhibitor may be administered or formulated in combination with a RAS(ON) GTP hydrolysis-promoting compound and/or any additional therapeutic agent described herein. AXL is a receptor tyrosine kinase that belongs to the TAM family of receptors, which also includes TYRO3 and MERTK. In some embodiments, an AXL inhibitor is one or more of bemcentib, BGB324, R428, SGI-7079, TP-0903, BMS-777607, UNC2025, and TP-0903. In some embodiments, reference to the term AXL inhibitor includes any such AXL inhibitor disclosed in any one of the following patent applications: WO 2023045816, WO 2022237843, WO 2022246179, WO 2021012717, WO 2021088787, WO 2021067772, WO 2021239133, WO 2021204713, WO 2020238802, WO 2019039525, WO 2019101178, WO 2019074116, WO 2017146236, WO 2016097918, WO 2015012298, WO 2010005876, WO 2010083465, CN 115073367, and JP 2022171109, each of which is incorporated herein by reference in its entirety, including the compound structures disclosed therein which are specifically incorporated herein by reference.

### V) IGFR Inhibitors

In some embodiments, compositions and methods described herein may include one or more insulin-like growth factor receptor 1 (IGF-1R) inhibitors. An IGFR inhibitor may be administered or formulated in combination with a RAS(ON) GTP hydrolysis-promoting compound and/or any additional therapeutic agent described herein. IGFR inhibitors have been developed to target the IGFR receptor, which plays a critical role in cancer progression and metastasis. In some embodiments, an IGFR inhibitor is one or more of linsitinib, AXL1717, OSI-906 (Linsitinib), BMS-754807, BI 836845, AZ12253801, PQIP (Pyrrolo[1,2-a]quinoxaline), and NVP-AEW541. In some embodiments, reference to the term IGFR inhibitor includes any such IGFR inhibitor disclosed in any one of the following patent applications: WO 2022115946, WO 2022217923, WO 2021203861, WO 2021246413, WO 2020116398, WO 2019046600, WO 2018195250, WO 2018221521, WO 2018204872, WO 2017072196, WO 2016173682, WO 2015162291, WO 2015162292, WO 2010066868, WO 2006069202, and CN 112125916, each of which is incorporated herein by reference in its entirety, including the compound structures disclosed therein which are specifically incorporated herein by reference.

### v) RET Inhibitors

In some embodiments, compositions and methods described herein may include one or more Rearranged during transfection (RET) inhibitors. An RET inhibitor may be administered or formulated in combination with a RAS(ON) GTP hydrolysis-promoting compound and/or any additional therapeutic agent described herein. RET plays a critical role in various cellular processes, including cell growth, differentiation, survival, and migration. RET is activated by binding of its ligands, such as glial cell line-derived neurotrophic factor (GDNF) family ligands, which leads to the activation of downstream signaling pathways that promote these cellular processes. In some embodiments, a RET inhibitor is one or more of pralsetinib, selpercatinib (LOXO—292), BLU-667, RXDX-105, TPX-0046, GSK3179106, molidustat (BAY 85-3934), and RPI-1 (Retrophin). In some embodiments, reference to the term RET inhibitor includes any such RET inhibitor disclosed in any one of the following patent applications: WO 2021211380, WO 2021057963, WO 2021043209, WO 2021222017, WO 2020035065, WO 2020114487, WO 2020200314, WO 2020200316, WO 2020114494, WO 2018071447, WO 2018213329, WO 2017079140, WO 2014050781, CN 113943285, CN 113683610, CN 113683611, CN 113620944, CN 113620945, CN 113527291, CN 113527292, CN 113527290, CN 113135896, CN 111057075, CN111233899, and CN111362923, each of which is incorporated herein by reference in its entirety, including the compound structures disclosed therein which are specifically incorporated herein by reference.

### vi) ROS1 Inhibitors

In some embodiments, compositions methods described herein may include one or more c-ros oncogene 1 (ROS1) inhibitors. A ROS1 inhibitor may be administered or formulated in combination with RAS(ON) GTP hydrolysis-promoting compound and/or any additional therapeutic agent described herein. ROS1 is a receptor tyrosine kinase that belongs to the insulin receptor family and plays a role in various cellular processes, including cell growth, differentiation, survival, and migration. In some embodiments, a ROS1 inhibitor is one or more of taletrectinib, DS-6051b, TPX-0131, GZD824, and PF-06463922. In some embodiments, reference to the term ROS1 inhibitor includes any such ROS1 inhibitor disclosed in any one of the following patent applications: WO 2021098703, WO 2020024825, and US 2017079972, each of which is incorporated herein by reference in its entirety, including the compound structures disclosed therein which are specifically incorporated herein by reference.

vii) PDGFR Inhibitors

In some embodiments, compositions and methods described herein may include one or more platelet-derived growth factor receptor (PDGFR) inhibitors. A PDGFR inhibitor may be administered or formulated in combination with RAS(ON) GTP hydrolysis-promoting compound and/or any additional therapeutic agent described herein. PDGFR is a family of receptor tyrosine kinases that consists of two members, PDGFRα and PDGFRβ. They are activated by binding to their ligands, such as platelet-derived growth factor (PDGF), which leads to the activation of downstream signaling pathways that promote cell growth, proliferation, and survival. In some embodiments, a PDGFR inhibitor is one or more of CP-673451, imatinib, nintedanib (ofev), sunitinib (sutent), pazopanib (votrient), regorafenib (stivarga), and dasatinib (sprycel).

viii) FGFR Inhibitors

In some embodiments, compositions and methods described herein may include one or more fibroblast growth factor receptor (FGFR) inhibitors. A FGFR inhibitor may be administered or formulated in combination with RAS(ON) GTP hydrolysis-promoting compound and/or any additional therapeutic agent described herein. FGFRs are a family of receptor tyrosine kinases that consists of four members, FGFR1-4. FGFRs are activated by binding to their ligands, fibroblast growth factors (FGFs), which leads to the activation of downstream signaling pathways that promote cell growth, differentiation, and survival. In some embodiments, the FGFR inhibitor is an inhibitor of FGFR2. In some embodiments, the FGFR inhibitor is an inhibitor of FGFR4. In some embodiments, a FGFR inhibitor is one or more of futibatinib (TAK-659), erdafitinib (balversa), infigratinib (Truseltiq), Debio 1347, and rogaratinib (BAY 1163877). In some embodiments, reference to the term FGFR inhibitor includes any such FGFR inhibitor disclosed in any one of the following patent applications: WO 2022033472, WO 2022152274, WO 2022166469, WO 2022206939, WO 2021037219, WO 2021089005, WO 2021113462, WO 2020185532, WO 2019213544, WO 2020164603, WO 2019154364, WO 2019034076, WO 2019213506, WO 2019223766, WO 2018028438, WO 2018153373, WO 2018121650, WO 2018010514, WO 2017028816, WO 2017118438, WO 2016134320, WO 2015008844, WO 2014172644, WO 2014007951, WO 2013179033, WO 2013087578, WO 2012047699, CN 105906630, CN 115869315, CN 115141176, CN 115043832, and CN 115028634, each of which is incorporated herein by reference in its entirety, including the compound structures disclosed therein which are specifically incorporated herein by reference.

### ix) VEGF Inhibitors

In some embodiments, compositions and methods described herein may include one or more vascular endothelial growth factor (VEGF) signaling inhibitors. VEGF (vascular endothelial growth factor) signaling inhibitors are a class of drugs that target the signaling pathway mediated by VEGF and its receptors. VEGF plays a critical role in angiogenesis, the process of forming new blood vessels from existing ones, and it is overexpressed in many types of cancer, making it an attractive target for cancer therapy. A VEGF inhibitor may be administered or formulated in combination with RAS(ON) GTP hydrolysis-promoting compound and/or any additional therapeutic agent described herein. In some embodiments, the VEGF inhibitor is an antibody or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto). In some embodiments, the VEGF inhibitor is one or more of bevacizumab, aflibercept, ramucirumab, sorafenib, sunitinib, and pazopanib.

### d) SHP Inhibitors

In some embodiments, compositions and methods described herein may include one or more SHP inhibitors. A SHP inhibitor may be administered or formulated in combination with a RAS(ON) GTP hydrolysis-promoting compound and/or any additional therapeutic agent described herein. In some embodiments, the SHP inhibitor is an inhibitor of SHP1. In some embodiments, the SHP inhibitor is an inhibitor of SHP2. In some embodiments, the SHP 1 inhibitor is SB6299 aka DA-4511. In some embodiments, a SHP2 inhibitor is one or more of SHP099, TNO155, RMC-4550, RMC-4630, JAB-3068, JAB-3312, RLY-1971, ERAS-601, SH3809, PF-07284892, or BBP-398. In some embodiments, reference to the term SHP2 inhibitor includes any such SHP2 inhibitor disclosed in any one of the following patent applications: WO 2023282702, WO 2023280283, WO 2023280237, WO 2023018155, WO 2023011513, WO 2022271966, WO 2022271964, WO 2022271911, WO 2022259157, WO 2022242767, WO 2022241975, WO 2022237676, WO 2022237367, WO 2022237178, WO 2022235822, WO 20222084008, WO 2022135568, WO 2022063190, WO 2022043865, WO 2022042331, WO 2022033430, WO 2022017444, WO 2022007869, WO 2021259077, WO 2021249449, WO 2021249057, WO 2021244659, WO 2021218755, WO 2021176072, WO 2021171261, WO 2021149817, WO 2021148010, WO 2021147879, WO 2021143823, WO 2021143701, WO 2021143680, WO 2021281752, WO 2021121397, WO 2021119525, WO 2021115286, WO 2021110796, WO 2021088945, WO 2021073439, WO 2021061706, WO 2021061515, WO 2021043077, WO 2021033153, WO 2021028362, WO 2021033153, WO 2021028362, WO 2021018287, WO 2020259679, WO 2020249079, WO 2020210384, WO 2020201991, WO 2020181283, WO 2020177653, WO 2020165734, WO 2020165733, WO 2020165732, WO 2020156243, WO 2020156242, WO 2020108590, WO 2020104635, WO 2020094104, WO 2020094018, WO 2020081848, WO 2020073949, WO 2020073945, WO 2020072656, WO 2020065453, WO 2020065452, WO 2020063760, WO 2020061103, WO 2020061101, WO 2020033828, WO 2020033286, WO 2020022323, WO 2019233810, WO 2019213318, WO 2019183367, WO 2019183364, WO 2019182960, WO 2019167000, WO 2019165073, WO 2019158019, WO 2019152454, WO 2019051469, WO 2019051084, WO 2018218133, WO 2018172984, WO 2018160731, WO 2018136265, WO 2018136264, WO 2018130928, WO 2018129402, WO 2018081091, WO 2018057884, WO 2018013597, WO 2017216706, WO 2017211303, WO 2017210134, WO 2017156397, WO 2017100279, WO 2017079723, WO 2017078499, WO 2016203406, WO 2016203405, WO 2016203404, WO 2016196591, WO 2016191328, WO 2015107495, WO 2015107494, WO 2015107493, WO 2014176488, WO 2014113584, CN 115677661, CN 115677660, CN 115611869, CN 115521305, CN 115490697, CN 115466273, CN 115394612, CN 115304613, CN 115304612, CN 115300513, CN 115197225, CN 114957162, CN 114920759, CN 114716448, CN 114671879, CN 114539223, CN 114524772, CN 114213417, CN 114195799, CN 114163457, CN 113896710, CN 113248521, CN 113248449, CN 113135924, CN 113024508, CN 112920131, CN 112823796, CN 112409334, CN 112402385, CN 112174935, 111848599, CN 111704611, CN 111393459, CN 111265529, CN 110143949, CN 108113848, U.S. Ser. No. 11/179,397, U.S. Ser. No. 11/044,675, U.S. Ser. No. 11/034,705, U.S. Ser. No. 11/033,547, U.S. Ser. No. 11/001,561, U.S. Ser. No. 10/988,466, U.S. Ser. No. 10/954,243, U.S. Ser. No. 10/934,302, or U.S. Ser. No. 10/858,359, each of which is incorporated herein by reference in its entirety, including the compound structures disclosed therein which are specifically incorporated herein by reference.

### e) SOS1 Inhibitors

In some embodiments, compositions and methods described herein may include one or more SOS1 inhibitors. A SOS1 inhibitor may be administered or formulated in combination with RAS(ON) GTP hydrolysis-promoting compound and/or any additional therapeutic agent described herein. In some embodiments, a SOS1 inhibitor is one or more of RMC-5845, RMC-4948, RMC-0331, BI-1701963, BI-3406, SDR5, MRTX-0902, and BAY-293. In some embodiments, reference to the term SOS1 inhibitor includes any such SOS1 inhibitor disclosed in any one of the following patent applications: WO 2023029833, WO 2023041049, WO 2023022497, WO 2022184116, WO 2022170952, WO 2022170917, WO 2022171184, WO 2022170802, WO 2022161461, WO 2022121813, WO 2022028506, WO 2022139304, WO 2021228028, WO 2019122129, CN 115215847, CN 115028644, CN 114685488, CN 111393519, each of which is incorporated herein by reference in its entirety, including the compound structures disclosed therein which are specifically incorporated herein by reference.

### f) Pharmaceutical Compositions

The disclosure provides pharmaceutical compositions including one or more RAS(ON) GTP hydrolysis-promoting compounds in combination with one or more RAS(OFF) inhibitors, or a pharmaceutically acceptable salt thereof, as active agents, and a pharmaceutically acceptable excipient.

In some embodiments, a compound is present in a pharmaceutical composition in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained release formulation; topical application, for example, as a cream, ointment, or a controlled release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Compounds described herein, whether expressly stated or not, may be provided or utilized in salt form, e.g., a pharmaceutically acceptable salt form, unless expressly stated to the contrary.

The compounds of the disclosure may have ionizable groups so as to be capable of preparation as pharmaceutically acceptable salts. These salts may be acid addition salts involving inorganic or organic acids or the salts may, in the case of acidic forms of the compounds of the disclosure, be prepared from inorganic or organic bases. In some embodiments, the compounds are prepared or used as pharmaceutically acceptable salts prepared as addition products of pharmaceutically acceptable acids or bases. Suitable pharmaceutically acceptable acids and bases are well known in the art, such as hydrochloric, sulfuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2 optionally substituted hydroxyl ethanesulfonate, lactobionate, lactate, laurate, lauryl sulfate, malate, maleate, malonate, methanesulfonate, 2 naphthalenesulfonate, nicotinate, nitrate, oleate, oxalate, palmitate, pamoate, pectinate, persulfate, 3 phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

For use as treatment of subjects, the compounds of the disclosure, or a pharmaceutically acceptable salt thereof, can be formulated as pharmaceutical or veterinary compositions. Depending on the subject to be treated, the mode of administration, and the type of treatment desired, e.g., prevention, prophylaxis, or therapy, the compounds, or a pharmaceutically acceptable salt thereof, are formulated in ways consonant with these parameters. A summary of such techniques may be found in Remington: The Science and Practice of Pharmacy, 21st Edition, Lippincott Williams & Wilkins, (2005); and Encyclopedia of Pharmaceutical Technology, eds. J. Swarbrick and J. C. Boylan, 1988 1999, Marcel Dekker, New York, each of which is incorporated herein by reference.

Compositions can be prepared according to conventional mixing, granulating or coating methods, respectively, and the present pharmaceutical compositions can contain from about 0.1% to about 99%, from about 5% to about 90%, or from about 1% to about 20% of a compound of the present disclosure, or pharmaceutically acceptable salt thereof, by weight or volume. In some embodiments, compounds, or a pharmaceutically acceptable salt thereof, described herein may be present in amounts totaling 1 95% by weight of the total weight of a composition, such as a pharmaceutical composition.

The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. A formulation will generally include a diluent as well as, in some cases, adjuvants, buffers, preservatives and the like. Compounds, or a pharmaceutically acceptable salt thereof, can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677.

Systemic administration may also include relatively noninvasive methods such as the use of suppositories, transdermal patches, transmucosal delivery and intranasal administration. Oral administration is also suitable for compounds of the disclosure, or a pharmaceutically acceptable salt thereof. Suitable forms include syrups, capsules, and tablets, as is understood in the art.

Each compound, or a pharmaceutically acceptable salt thereof, as described herein, may be formulated in a variety of ways that are known in the art. For example, the first and second agents of the combination therapy may be formulated together or separately. Other modalities of combination therapy are described herein.

The individually or separately formulated agents can be packaged together as a kit. Non limiting examples include, but are not limited to, kits that contain, e.g., two pills, a pill and a powder, a suppository and a liquid in a vial, two topical creams, etc. The kit can include optional components that aid in the administration of the unit dose to subjects, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one subject, multiple uses for a particular subject (at a constant dose or in which the individual compounds, or a pharmaceutically acceptable salt thereof, may vary in potency as therapy progresses); or the kit may contain multiple doses suitable for administration to multiple subjects (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, optionally substituted hydroxylpropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion-controlled release can be achieved by appropriate coating of a tablet, capsule, pellet, or granulate formulation of compounds, or by incorporating the compound, or a pharmaceutically acceptable salt thereof, into an appropriate matrix. A controlled release coating may include one or more of the coating substances mentioned above or, e.g., shellac, beeswax, glycowax, castor wax, carnauba wax, stearyl alcohol, glyceryl monostearate, glyceryl distearate, glycerol palmitostearate, ethylcellulose, acrylic resins, dl polylactic acid, cellulose acetate butyrate, polyvinyl chloride, polyvinyl acetate, vinyl pyrrolidone, polyethylene, polymethacrylate, methylmethacrylate, 2 optionally substituted hydroxylmethacrylate, methacrylate hydrogels, 1,3 butylene glycol, ethylene glycol methacrylate, or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate methyl methacrylate, polyvinyl chloride, polyethylene, or halogenated fluorocarbon.

The liquid forms in which the compounds, or a pharmaceutically acceptable salt thereof, and compositions of the present disclosure can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Generally, when administered to a human, the oral dosage of any of the compounds of the disclosure, or a pharmaceutically acceptable salt thereof, will depend on the nature of the compound, and can readily be determined by one skilled in the art. A dosage may be, for example, about 0.001 mg to about 2000 mg per day, about 1 mg to about 1000 mg per day, about 5 mg to about 500 mg per day, about 100 mg to about 1500 mg per day, about 500 mg to about 1500 mg per day, about 500 mg to about 2000 mg per day, or any range derivable therein.

In some embodiments, the pharmaceutical composition may further include an additional compound having antiproliferative (e.g., anti-cancer) activity. Depending on the mode of administration, compounds, or a pharmaceutically acceptable salt thereof, will be formulated into suitable compositions to permit facile delivery. Each compound, or a pharmaceutically acceptable salt thereof, of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

It will be appreciated that the compounds and pharmaceutical compositions of the present disclosure can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics or procedures and the desired therapeutic effect to be achieved. It will also be appreciated that the therapies employed may achieve a desired effect for the same disorder, or they may achieve different effects (e.g., control of any adverse effects).

Administration of each drug in a combination therapy, as described herein, can, independently, be one to four times daily for one day to one year, and may even be for the life of the subject. Chronic, long-term administration may be indicated.

### II. Methods

In one aspect, the present disclosure is directed to methods of treating a disease or disorder that is characterized by aberrant RAS activity (e.g., cancer or a RASopathy). In some embodiments the disease or disorder is cancer (e.g., a cancer having one or more RAS mutations that cause aberrant RAS activity). In the preceding embodiment, the method generally comprises administering to the subject a therapeutically effective amount of a RAS(ON) GTP hydrolysis-promoting compound and a RAS(OFF) inhibitor. Suitable RAS(ON) GTP hydrolysis-promoting compounds and additional therapeutic agents useful in the methods disclosed herein are described in section I and incorporated into this section by reference.

Accordingly, the disclosure provides methods of treating cancer in a subject in need thereof, the methods including administering to the subject a therapeutically effective amount of one or more RAS(ON) GTP hydrolysis-promoting compounds described herein, or a pharmaceutically acceptable salt thereof, or a pharmaceutical composition including one or more RAS(ON) GTP hydrolysis-promoting compounds described herein or salts thereof.

The disclosure also provides a method of treating cancer in a subject in need thereof, wherein the cancer includes a mutation in RAS. In one embodiment, the addition of a RAS(ON) GTP hydrolysis-promoting compound, or a pharmaceutically acceptable salt or pharmaceutical composition thereof, synergistically increases the activity of a RAS(OFF) inhibitor, or a pharmaceutically acceptable salt or pharmaceutical composition thereof. Any method for determining whether two compounds exhibit synergy may be used for determining the synergistic effect of the combination, such as methods described herein.

Several mathematical models have been developed to determine whether two compounds act synergistically, i.e., beyond a mere additive effect. For instance, Loewe Additivity (Loewe (1928) Physiol. 27: 47-187), Bliss Independence (Bliss (1939) Ann. Appl. Biol. 26: 585-615), Highest Single Agent, ZIP (Yadav et al (2015) Comput Struct Biotech J 13: 504-513) and other models (Chou & Talalay (1984) Adv Enzyme Regul 22: 27-55. #6382953; and Greco et al. (1995) Pharmacol Rev 47(2): 331-85. #7568331) are well known models in the pharmaceutical industry and may be used to calculate a “synergy score” that indicates whether synergy was detected and the magnitude of such synergy. Additional models for determining synergy of two compounds can be found in the examples below.

In general, the mathematical models use data obtained from single agent values to determine the predicted additive effect of the combination which is compared to the observed effect for the combination. If the observed effect is greater than the predicted effect, the combination is deemed to be synergistic. For example, the Bliss independence model compares the observed combination response (Yo) with the predicted combination response (Yp), which was obtained based on the assumption that there is no effect from drug-drug interactions. Typically, the combination effect is declared synergistic if Yo is greater than Yp.

In some embodiments, “synergistic effect” as used herein refers to combination of a RAS(ON) GTP hydrolysis-promoting compound or a pharmaceutically acceptable salt thereof, and an additional therapeutic agent (e.g., a RAS(OFF) inhibitor) or a pharmaceutically acceptable salt thereof producing an effect, for example, any of the beneficial or desired results including in vitro results as well as clinical results or endpoints as described herein, which is greater than the sum of the effect observed when a RAS(ON) GTP hydrolysis-promoting compound or a pharmaceutically acceptable salt thereof and an additional therapeutic agent (e.g., RAS(OFF) inhibitor) or a pharmaceutically acceptable salt thereof are administered alone. The stronger the hydrolyzing capability of a RAS(ON) GTP hydrolysis-promoting compound, the greater the synergistic effect seen with a RAS(OFF) inhibitor.

In some embodiments, the disclosure provides methods of treating cancer in a subject in need thereof, the methods including administering to the subject a therapeutically effective amount of one or more RAS(ON) GTP hydrolysis-promoting compounds described herein, or a pharmaceutically acceptable salt thereof, or a pharmaceutical composition and a receptor tyrosine kinase (RTK) inhibitor.

In some embodiments, the disclosure provides methods of treating cancer in a subject in need thereof, the methods including administering to the subject a therapeutically effective amount of one or more RAS(ON) GTP hydrolysis-promoting compounds described herein, or a pharmaceutically acceptable salt thereof, or a pharmaceutical composition and a SHP2 inhibitor.

In some embodiments, the disclosure provides methods of treating cancer in a subject in need thereof, the methods including administering to the subject a therapeutically effective amount of one or more RAS(ON) GTP hydrolysis-promoting compounds described herein, or a pharmaceutically acceptable salt thereof, or a pharmaceutical composition and a SOS1 inhibitor.

The RAS(ON) GTP hydrolysis-promoting compound and the additional therapeutic agent may be administered simultaneously or sequentially. The RAS(ON) GTP hydrolysis-promoting compound and the RAS(OFF) inhibitor may be administered as a single formulation or in separate formulations. In some embodiments, the RAS(ON) GTP hydrolysis-promoting compound is administered for a first period of time; and the additional therapeutic agent is administered for a second period of time, wherein the first period of time and the second period of time do not overlap and the first period of time precedes the second period of time; and the additional therapeutic agent and RAS(ON) GTP hydrolysis-promoting compound are administered for a second period of time, wherein the first period of time and the second period of time do not overlap and the first period of time precedes the second period of time.

In some embodiments, the cancer is colorectal cancer, non-small cell lung cancer, small-cell lung cancer, pancreatic cancer, appendiceal cancer, melanoma, acute myeloid leukemia, small bowel cancer, ampullary cancer, germ cell cancer, cervical cancer, cancer of unknown primary origin, endometrial cancer, esophagogastric cancer, GI neuroendocrine cancer, ovarian cancer, sex cord stromal tumor cancer, hepatobiliary cancer, or bladder cancer. In some embodiments, the cancer is appendiceal, endometrial or melanoma. In some embodiments, the cancer is non-small cell lung cancer. In some embodiments, the cancer is pancreatic cancer.

In some embodiments, the compounds of the present disclosure or pharmaceutically acceptable salts thereof, pharmaceutical compositions including such compounds or salts, and methods provided herein may be used for the treatment of a wide variety of cancers such as astrocytic, breast, cervical, colorectal, endometrial, esophageal, gastric, head and neck, hepatocellular, laryngeal, lung, oral, ovarian, prostate and thyroid carcinomas and sarcomas. Other cancers include, for example: Cardiac, for example: sarcoma (angiosarcoma, fibrosarcoma, rhabdomyosarcoma, liposarcoma), myxoma, rhabdomyoma, fibroma, lipoma and teratoma; Lung, for example: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal, for example: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, lipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract, for example: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver, for example: hepatoma (hepatocellular carcinoma), cholangiocarcinoma, hepatoblastoma, angiosarcoma, hepatocellular adenoma, hemangioma; Biliary tract, for example: gall bladder carcinoma, ampullary carcinoma, cholangiocarcinoma; Bone, for example: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochondroma (osteocartilaginous exostoses), benign chondroma, chondroblastoma, chondromyxofibroma, osteoid osteoma and giant cell tumors; Nervous system, for example: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, neurofibromatosis type 1, meningioma, glioma, sarcoma); Gynecological, for example: uterus (endometrial carcinoma, uterine carcinoma, uterine corpus endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic, for example: blood (myeloid leukemia (acute and chronic), acute lymphoblastic myeloproliferative neoplasms), multiple myeloma, myelodysplastic syndrome), Hodgkin's disease, non-Hodgkin's lymphoma (malignant lymphoma); Skin, for example: malignant melanoma, basal cell carcinoma, squamous cell carcinoma, Kaposi's sarcoma, moles dysplastic nevi, lipoma, angioma, dermatofibroma, keloids, psoriasis; and Adrenal glands, for example: neuroblastoma.

In some embodiments, the cancer includes a RAS mutation, such as a RAS mutation described herein. In some embodiments, a mutation is selected from: the following KRAS mutants: G12D, G12V, G12C, G13D, G12R, G12A, G12S, A146T, G13C, K117N, A146V, G12F, L19F, Q22K, V14I, A59T, A146P, G13R, G12L, or G13V, and combinations thereof; the following HRAS mutants: G13R, G12S, G12D, G13V, G13D, G12C, K117N, A59T, G12V, G13C, G13S, A18V, D119N, G13N, A146T, A66T, G12A, A146V, G12N, or G12R, and combinations thereof; and the following NRAS mutants: G12D, G13R, G13D, G12S, G12C, G12V, G12A, G13V, G12R, P185S, G13C, A146T, G60E, A59D, E132K, E49K, T501, A146V, or A59T, and combinations thereof; or a combination of any of the foregoing. In some embodiments, the cancer includes a KRAS mutation selected from the group consisting of G12C, G12D, G13C, G12V, G13D, G12R, and G12S. In some embodiments, the cancer includes an NRAS mutation at G12C. In some embodiments, the cancer includes a RAS mutation selected from the group consisting of G12C, G13C, G12A, G12D, G13D, G12S, G13S, G12V and G13V. In some embodiments, the cancer includes at least two RAS mutations selected from the group consisting of G12C, G13C, G12A, G12D, G13D, G12S, G13S, G12V and G13V.

In some embodiments, a compound of the present disclosure binds to or inhibits more than one RAS mutant. In some embodiments, a compound may inhibit both KRAS G12D and KRAS G12V. In some embodiments, a compound may bind to or inhibit both KRAS G12V and KRAS G12S. In some embodiments, a compound of the present disclosure binds to or inhibits wild-type RAS in addition to a RAS mutant. In some embodiments, a compound of the present disclosure binds to or inhibits RAS^{amp} in addition to one or more additional RAS mutations (e.g., K-, H- or N-RAS^{amp} and KRAS G12D, G12V, G12C, G13D, G12R, G12A, G12S, A146T, G13C, K117N, A146V, G12F, L19F, Q22K, V14I, A59T, A146P, G13R, G12L, or G13V; K-, H- or N-RAS^{amp} and HRAS, G13R, G12S, G12D, G13V, G13D, G12C, K117N, A59T, G12V, G13C, G13S, A18V, D119N, G13N, A146T, A66T, G12A, A146V, G12N, or G12R; or K-, H- or N-RAS^{amp} and NRAS G12D, G13R, G13D, G12S, G12C, G12V, G12A, G13V, G12R, P185S, G13C, A146T, G60E, A59D, E132K, E49K, T501, A146V, or A59T).

In some embodiments, the cancer is non-small cell lung cancer, and the RAS mutation includes a KRAS mutation, such as KRAS G12C, KRAS G12V or KRAS G12D. In some embodiments, the cancer is colorectal cancer, and the RAS mutation includes a KRAS mutation, such as KRAS G12C, KRAS G12V or KRAS G12D. In some embodiments, the cancer is pancreatic cancer, and the RAS mutation includes an NRAS mutation, such as NRAS G12D. In some embodiments, the cancer is melanoma.

In some embodiments, a cancer includes a RAS mutation and an STK11LoF, a KEAP1, an EPHA5 or an NF1 mutation. In some embodiments, the cancer is non-small cell lung cancer and includes a KRAS G12C mutation. In some embodiments, the cancer is non-small cell lung cancer and includes a KRAS G12C mutation and an STK11LoF mutation. In some embodiments, the cancer is non-small cell lung cancer and includes a KRAS G12C mutation and an STK11LoF mutation. In some embodiments, a cancer includes a KRAS G13C RAS mutation and an STK11LoF, a KEAP1, an EPHA5 or an NF1 mutation. In some embodiments, the cancer is non-small cell lung cancer and includes a KRAS G12D mutation. In some embodiments, the cancer is non-small cell lung cancer and includes a KRAS G12V mutation. In some embodiments, the cancer is colorectal cancer and includes a KRAS G12C mutation. In some embodiments, the cancer is pancreatic cancer and includes a KRAS G12D mutation. In some embodiments, the cancer is pancreatic cancer and includes a KRAS G12V mutation. In some embodiments, the cancer is pancreatic cancer and includes a KRAS G12R mutation. In some embodiments, the cancer is endometrial cancer and includes a KRAS G12C mutation. In some embodiments, the cancer is gastric cancer and includes a KRAS G12C mutation.

Methods for detecting a mutation in a KRAS, HRAS or NRAS nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for G12C KRAS, HRAS or NRAS mutations by real-time PCR. In real-time PCR, fluorescent probes specific for the KRAS, HRAS or NRAS G12C mutation are used. When a mutation is present, the probe binds and fluorescence is detected. In some embodiments, the KRAS, HRAS or NRAS G12C mutation is identified using a direct sequencing method of specific regions (e.g., exon 2 or exon 3) in the KRAS, HRAS or NRAS gene. This technique will identify all possible mutations in the region sequenced.

Methods for detecting a mutation in a KRAS, HRAS or NRAS protein are known by those of skill in the art. These methods include, but are not limited to, detection of a KRAS, HRAS or NRAS mutant using a binding agent (e.g., an antibody) specific for the mutant protein, protein electrophoresis and Western blotting, and direct peptide sequencing. Other methods include ctDNA measurement (e.g., Cescon et al., Nature Cancer 1:276-290 (2020)), and utilization of a high-sensitivity diagnostic assay (with CE-IVD mark), e.g., as described in Domagala, et al., Pol J Pathol 3: 145-164 (2012), incorporated herein by reference in its entirety, including TheraScreen PCR; AmoyDx; PNACIamp; RealQuality; EntroGen; LightMix; StripAssay; Hybcell plexA; Devyser; Surveyor; Cobas; and TheraScreen Pyro. See, also, e.g., WO 2020/106640.

Methods for determining whether a tumor or cancer includes a G12C or other KRAS, HRAS or NRAS mutation can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a (CTC) sample. In some embodiments, the sample is processed to a cell lysate. In some embodiments, the sample is processed to DNA or RNA.

Also provided is a method of inhibiting or binding to a RAS protein in a cell, the method including contacting the cell with an effective amount of a combination of a RAS(ON) GTP hydrolysis-promoting compound of the present disclosure, or a pharmaceutically acceptable salt thereof, and optionally an additional therapeutic agent (e.g., a RAS(OFF) inhibitor, RTK inhibitor, SHP2 inhibitor, or SOS1 inhibitor), or a pharmaceutically acceptable salt thereof. The cell may be a cancer cell. The cancer cell may be of any type of cancer described herein. The cell may be in vivo or in vitro.

In some embodiments of any of the methods described herein, before treatment with the compositions or methods of the invention, the patient was treated with one or more of a chemotherapy, a targeted anticancer agent, radiation therapy, and surgery, and optionally, the prior treatment was unsuccessful; and/or the patient has been administered surgery and optionally, the surgery was unsuccessful; and/or the patient has been treated with a platinum-based chemotherapeutic agent, and optionally, the patient has been previously determined to be non-responsive to treatment with the platinum-based chemotherapeutic agent; and/or the patient has been treated with a kinase inhibitor, and optionally, the prior treatment with the kinase inhibitor was unsuccessful; and/or the patient was treated with one or more other therapeutic agent(s).

In various embodiments, the disclosure provides a method of treating cancer in a subject comprising administering to the subject a RAS(ON) GTP hydrolysis-promoting compound or combination with optionally an additional therapeutic agent (e.g., a RAS(OFF) inhibitor, RTK inhibitor, SHP2 inhibitor, or SOS1 inhibitor) as described herein, wherein the subject has one or more tumors that are resistant or unresponsive to treatment. In various embodiments, the subject has one or more tumors that are resistant or unresponsive to one or more treatments selected from the group consisting of surgery, radiation, chemotherapy, biologic agents, small molecules, cell-based therapy, hormone therapy, and immunotherapy. In various embodiments, treatment is a standard of care therapy, first-line therapy, second-line therapy, or third-line therapy. In various embodiments, the subject has one or more tumors that have progressed during one or more treatments, wherein the treatments are standard of care therapy, first-line therapy, second-line therapy, or third-line therapy.

First-line therapy is defined as a treatment that is administered to a subject suffering from cancer who has not received any prior treatment. Second-line therapy is defined as treatment that is administered to a subject suffering from cancer who has received prior first-line therapy but experienced disease progression during first-line treatment. Third-line therapy is defined as treatment that is administered to a subject suffering from cancer who has received prior first and second-line treatment but has experienced disease progression during second-line treatment. Each particular type of cancer has a first-line, second-line, and third-line therapy. The first-, second-, and third-line therapies for types of cancer are known in the art. In addition, FDA approved drug labels will indicate if a particular drug is approved as a first-, second-, or third-line therapy.

Several criteria and definitions published in the literature can be used to determine the effect of one or more treatments on tumors in a subject suffering from cancer. Based on these criteria, tumors are defined as “responsive,” “stable,” or “progressive” when they improve, remain the same, or worsen during treatment, respectively.

Examples of the commonly used criteria published in the literature include Response Evaluation Criteria in Solid Tumors (RECIST), Modified Response Evaluation Criteria in Solid Tumors (mRECIST), PET Response Criteria in Solid Tumors (PERCIST), Choi Criteria, Lugano Response Criteria, European Association for the Study of the Liver (EASL) Criteria, Response Evaluation Criteria in the Cancer of the Liver (RECICL), and WHO Criteria in Tumor Response.

In various embodiments, the disclosure provides a method of treating cancer in a subject comprising administering to the subject a RAS(ON) GTP hydrolysis-promoting compound of the present disclosure, or a pharmaceutically acceptable salt thereof, and optionally an additional therapeutic agent (e.g., a RAS(OFF) inhibitor, RTK inhibitor, SHP2 inhibitor, or SOS1 inhibitor) as described herein, wherein the subject cannot tolerate standard of care therapy, first-line therapy, second-line therapy, or third-line therapy. In various embodiments, the disclosure provides a method of treating cancer in a subject comprising administering to the subject a RAS(ON) GTP hydrolysis-promoting compound of the present disclosure, or a pharmaceutically acceptable salt thereof, and optionally an additional therapeutic agent (e.g., a RAS(OFF) inhibitor, RTK inhibitor, SHP2 inhibitor, or SOS1 inhibitor) as described herein, wherein the subject has experienced tumor recurrence after surgical resection of the primary tumor. In various embodiments, the disclosure provides a method of treating cancer in a subject comprising administering to the subject a RAS(ON) GTP hydrolysis-promoting compound of the present disclosure, or a pharmaceutically acceptable salt thereof, and optionally an additional therapeutic agent (e.g., a RAS(OFF) inhibitor, RTK inhibitor, SHP2 inhibitor, or SOS1 inhibitor) as described herein, wherein the subject has a tumor that cannot be surgically removed. In various embodiments, the disclosure provides a method of treating cancer in a subject comprising administering to the subject a RAS(ON) GTP hydrolysis-promoting compound of the present disclosure, or a pharmaceutically acceptable salt thereof, and optionally an additional therapeutic agent (e.g., a RAS(OFF) inhibitor, RTK inhibitor, SHP2 inhibitor, or SOS1 inhibitor) as described herein, wherein the subject has no treatment options available.

Several therapies used in the treatment of cancer (e.g., chemotherapies) are cytotoxic and are associated with significant side-effects and toxicities that are associated with poor outcomes and poor response to treatment. Prior to administering such treatments, clinicians rely on several assessment tools to help determine the risk of a subject suffering from cancer experiencing treatment related toxicities and adverse events. Based on the results of these assessments, a subject suffering from cancer is considered intolerant to therapy if they are determined to be at increased risk of experiencing therapy-related toxicities and adverse events resulting in poor outcomes. Examples of commonly used assessment tools used in the determination of therapy intolerance include Karnofsky Performance Status (KPS), Eastern Cooperative Oncology Group Performance Status (ECOG PS), Timed Get Up and Go (TUG), Short Physical Performance Battery (SPPB), Comprehensive Geriatric Assessment (CGA), Cancer Aging Research Group (CARG) Score, and Chemotherapy Risk Assessment Scale for High-Age Patients (CRASH).

In some embodiments, the subject's cancer progression is reduced or prevented. Disease progression of a cancer (e.g., a cancer described herein) can be evaluated by any one or more of several established methods. A person of skill in the art can monitor a subject by direct observation in order to evaluate how the symptoms exhibited by the subject have changed (e.g., a decrease or absence of symptoms) in response to a treatment (e.g., a method of treatment disclosed herein). A subject may also be examined by MRI, CT scan, or PET analysis in order to determine if a tumor has metastasized or if the size of a tumor has changed (e.g., decreased in response to a treatment (e.g., a method of treatment described herein)). Optionally, cells can be extracted from the subject through a biopsy or procedure or tumor DNA can be isolated from the blood of a subject, and a quantitative biochemical analysis can be conducted in order to assess the relative cancer burden and determine the presence or emergence of specific mutations possibly involved in resistance. Based on the results of these analyses, a person of skill in the art may prescribe higher/lower dosages or more/less frequent dosing of a treatment in subsequent rounds of treatment.

In various embodiments, the disclosure provides a method of treating cancer in a subject comprising administering to the subject a RAS(ON) GTP hydrolysis-promoting compound and optionally an additional therapeutic agent (e.g., a RAS(OFF) inhibitor, RTK inhibitor, SHP2 inhibitor, or SOS1 inhibitor). In various embodiments, the administering reduces tumor size or inhibits tumor growth. In various embodiments, the administering induces tumor cell death, apoptosis, or necrosis.

It is contemplated that the methods herein reduce tumor size or tumor burden in the subject or reduce metastasis in the subject. In various embodiments, the methods reduce the tumor size by 10%, 20%, 30% or more. In various embodiments, the methods reduce tumor size by about or at least about 10%, 15%, 20%, 25%, 30%, 35%, 40%, 45%, 50%, 55%, 60%, 65%, 70%, 75%, 80%, 85%, 90%, or 95% or about 100%, or including all values and ranges that lie in between these values.

In one aspect, the present disclosure provides methods for treating a RAS protein-related disorder in a subject where the RAS-related disorder pathology is mediated, in part, through increased signaling in the RAS/MAPK pathway. In various embodiments, the method generally comprises administering to the subject a therapeutically effective amount of a RAS(ON) GTP hydrolysis-promoting compound. In some embodiments, the RAS protein-related disorder is a RASopathy. A RASopathy is a group of genetic disorders that are caused by mutations in genes involved in the RAS/MAPK signaling pathway. RASopathies are characterized by a range of clinical features and can affect multiple organ systems, including the cardiovascular, musculoskeletal, neurological, and dermatological systems.

In one aspect, the present disclosure is directed to methods of treating a disease or disorder that is characterized by aberrant RAS activity (e.g., cancer or a RASopathy). In some embodiments the disease or disorder is cancer (e.g., a cancer having one or more RAS mutations that cause aberrant RAS activity). Non-limiting examples of non-cancerous RAS related diseases or disorders are shown in Table 3. In each embodiment, the method generally comprises administering to the subject a therapeutically effective amount of a RAS(ON) GTP hydrolysis-promoting compound. In some embodiments, the methods comprise administering RAS(ON) GTP hydrolysis-promoting compound in combination with one or more therapeutic agents. Suitable RAS(ON) GTP hydrolysis-promoting compounds and additional therapeutic agents are described herein.

TABLE 3
Exemplary RAS related Non-cancerous Indications

| Disease or disorder | | References |
|---|---|---|
| Immune disease | Autoimmune disease | Journal of Clinical Immunology vol. 35, |
| | | pp. 454-458 (2015) |
| | Rheumatoid arthritis | The Open Rheumatoid Journal vol. 6, |
| | | pp. 259-272 (2012) |
| | RAS-related autoimmune | PNAS vol. 104, pp. 8953-8958 (2007) |
| | lymphoproliferative | Blood vol. 117, pp. 2887-2890 (2011) |
| | disorders | |
| Infection | Influenza | Cancer Research vol. 61, |
| | | pp. 8188-8193 (2001) |
| | | PloS ONE vol. 6, el6324 (2011) |
| | | Seikagaku: The Journal of the Japanese |
| | | Biochemical Society vol. 87, Issue 1 |
| | EBV infection | Oncogene vol. 23, pp. 8619-8628 (2004) |
| | HIV infection | Journal of Biological Chemstry vol. 275, |
| | | pp. 16513-16517 (2000) |
| Neurologic disease | Alzheimer's disease | Biochimica et Biophysica Acta vol. 1802, |
| | | pp. 396-405 (2010) |
| | | Neurobiology of Disease vol. 43, |
| | | pp. 38-45 (2011) |
| | Parkinson's disease | Biochimica et Biophysica Acta vol. 1802, |
| | | pp. 396-405 (2010) |
| | ALS | Biochimica et Biophysica Acta vol. 1802, |
| | | pp. 396-405 (2010) |
| RAS/MAPK syndrome | Noonan Syndrome | Human Molecular Genetics vol. 15, |
| | | pp. R220-R226 (2006) |
| | Costello syndrome | Genetics in Medicine vol. 14, |
| | | pp. 285-292 (2012) |
| | CFC syndrome | Human Mutation vol. 29, pp. 992-1006 (2008) |
| Other diseases | Cirrhosis/Chronic | Gastroenterologia Japonica vol. 24, |
| or disorders | hepatitis | pp. 270-276 (1989) |
| | Memory impairment | Nature Communications vol. 7, 12926 (2016) |

In some embodiments, the methods include treating a RASopathy selected from Noonan syndrome, Costello syndrome, cardiofaciocutaneous syndrome, neurofibromatosis type 1, and Legius syndrome. While each RASopathy has unique features, they all share certain similarities, such as facial dysmorphisms, cardiac abnormalities, developmental delays, and an increased risk of certain cancers.

RASopathies are typically diagnosed through a combination of clinical evaluation, genetic testing, and imaging studies. Treatment and management of RASopathies depend on the specific type and severity of the disorder, but may include medication, surgery, and supportive therapies such as physical and occupational therapy.

### a) Combination Therapies

The methods of the disclosure may include a RAS(ON) GTP hydrolysis-promoting compound of the disclosure in combination an additional therapeutic agent (e.g., non-drug treatments or therapeutic agents). The dosages of one or more of the additional therapies (e.g., non-drug treatments or therapeutic agents) may be reduced from standard dosages when administered alone. For example, doses may be determined empirically from drug combinations and permutations or may be deduced by isobolographic analysis (e.g., Black et al., Neurology 65:S3-S6 (2005)).

A compound of the present disclosure may be administered before, after, or concurrently with one or more of such additional therapies. When combined, dosages of a compound of the disclosure and dosages of the one or more additional therapies (e.g., non-drug treatment or therapeutic agent) provide a therapeutic effect (e.g., synergistic or additive therapeutic effect). A compound of the present disclosure and an additional therapy, such as an anti-cancer agent, may be administered together, such as in a unitary pharmaceutical composition, or separately and, when administered separately, this may occur simultaneously or sequentially. Such sequential administration may be close or remote in time.

In some embodiments, the additional therapy is the administration of side-effect limiting agents (e.g., agents intended to lessen the occurrence or severity of side effects of treatment. For example, in some embodiments, the compounds of the present disclosure can also be used in combination with a therapeutic agent that treats nausea. Examples of agents that can be used to treat nausea include: dronabinol, granisetron, metoclopramide, ondansetron, and prochlorperazine, or pharmaceutically acceptable salts thereof.

In some embodiments, the one or more additional therapies includes a non-drug treatment (e.g., surgery or radiation therapy). In some embodiments, the one or more additional therapies includes a therapeutic agent (e.g., a compound or biologic that is an anti-angiogenic agent, signal transduction inhibitor, antiproliferative agent, glycolysis inhibitor, or autophagy inhibitor). In some embodiments, the one or more additional therapies includes a non-drug treatment (e.g., surgery or radiation therapy) and a therapeutic agent (e.g., a compound or biologic that is an anti-angiogenic agent, signal transduction inhibitor, antiproliferative agent, glycolysis inhibitor, or autophagy inhibitor). In other embodiments, the one or more additional therapies includes two therapeutic agents. In still other embodiments, the one or more additional therapies includes three therapeutic agents. In some embodiments, the one or more additional therapies includes four or more therapeutic agents.

In this Combination Therapy section, all references are incorporated by reference for the agents described, whether explicitly stated as such or not.

### i) Non-Drug Therapies

Examples of non-drug treatments include, but are not limited to, radiation therapy, cryotherapy, hyperthermia, surgery (e.g., surgical excision of tumor tissue), and T cell adoptive transfer (ACT) therapy.

In some embodiments, the compounds of the disclosure may be used as an adjuvant therapy after surgery. In some embodiments, the compounds of the disclosure may be used as a neo-adjuvant therapy prior to surgery.

Radiation therapy may be used for inhibiting abnormal cell growth or treating a hyperproliferative disorder, such as cancer, in a subject (e.g., mammal (e.g., human)). Techniques for administering radiation therapy are known in the art. Radiation therapy can be administered through one of several methods, or a combination of methods, including, without limitation, external-beam therapy, internal radiation therapy, implant radiation, stereotactic radiosurgery, systemic radiation therapy, radiotherapy, and permanent or temporary interstitial brachy therapy. The term “brachy therapy,” as used herein, refers to radiation therapy delivered by a spatially confined radioactive material inserted into the body at or near a tumor or other proliferative tissue disease site. The term is intended, without limitation, to include exposure to radioactive isotopes (e.g., At-211, 1-131, 1-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as 1-125, 1-131, Yb-169, 1r-192 as a solid source, 1-125 as a solid source, or other radionuclides that emit photons, beta particles, gamma radiation, or other therapeutic rays. The radioactive material can also be a fluid made from any solution of radionuclide(s), e.g., a solution of 1-125 or 1-131, or a radioactive fluid can be produced using a slurry of a suitable fluid containing small particles of solid radionuclides, such as Au-198, or Y-90. Moreover, the radionuclide(s) can be embodied in a gel or radioactive micro spheres.

In some embodiments, the compounds of the present disclosure can render abnormal cells more sensitive to treatment with radiation for purposes of killing or inhibiting the growth of such cells. Accordingly, this disclosure further relates to a method for sensitizing abnormal cells in a mammal to treatment with radiation which comprises administering to the mammal an amount of a compound of the present disclosure, which amount is effective to sensitize abnormal cells to treatment with radiation. The amount of the compound in this method can be determined according to the means for ascertaining effective amounts of such compounds described herein. In some embodiments, the compounds of the present disclosure may be used as an adjuvant therapy after radiation therapy or as a neo-adjuvant therapy prior to radiation therapy.

In some embodiments, the non-drug treatment is a T cell adoptive transfer (ACT) therapy. In some embodiments, the T cell is an activated T cell. The T cell may be modified to express a chimeric antigen receptor (CAR). CAR modified T (CAR-T) cells can be generated by any method known in the art. For example, the CAR-T cells can be generated by introducing a suitable expression vector encoding the CAR to a T cell. Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of T cell lines available in the art may be used. In some embodiments, the T cell is an autologous T cell. Whether prior to or after genetic modification of the T cells to express a desirable protein (e.g., a CAR), the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 7,572,631; 5,883,223; 6,905,874; 6,797,514; and 6,867,041.

### ii) Therapeutic Agents

A therapeutic agent may be a compound used in the treatment of cancer or symptoms associated therewith.

For example, a therapeutic agent may be a steroid. Accordingly, in some embodiments, the one or more additional therapies includes a steroid. Suitable steroids may include, but are not limited to, 21-acetoxypregnenolone, alclometasone, algestone, amcinonide, beclomethasone, betamethasone, budesonide, chloroprednisone, clobetasol, clocortolone, cloprednol, corticosterone, cortisone, cortivazol, deflazacort, desonide, desoximetasone, dexamethasone, diflorasone, diflucortolone, difuprednate, enoxolone, fluazacort, fiucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, mometasone furoate, paramethasone, prednicarbate, prednisolone, prednisolone 25-diethylaminoacetate, prednisolone sodium phosphate, prednisone, prednival, prednylidene, rimexolone, tixocortol, triamcinolone, triamcinolone acetonide, triamcinolone benetonide, triamcinolone hexacetonide, and salts or derivatives thereof.

Further examples of therapeutic agents that may be used in combination therapy with a compound of the present disclosure include compounds described in the following patents: U.S. Pat. Nos. 6,258,812, 6,630,500, 6,515,004, 6,713,485, 5,521,184, 5,770,599, 5,747,498, 5,990,141, 6,235,764, and 8,623,885, and International Patent Applications WO01/37820, WO01/32651, WO02/68406, WO02/66470, WO02/55501, WO04/05279, WO04/07481, WO04/07458, WO04/09784, WO02/59110, WO99/45009, WO00/59509, WO99/61422, WO00/12089, and WO00/02871.

A therapeutic agent may be a biologic (e.g., cytokine (e.g., interferon or an interleukin such as IL-2)) used in treatment of cancer or symptoms associated therewith. In some embodiments, the biologic is an immunoglobulin-based biologic, e.g., a monoclonal antibody (e.g., a humanized antibody, a fully human antibody, an Fc fusion protein, or a functional fragment thereof) that agonizes a target to stimulate an anti-cancer response or antagonizes an antigen important for cancer. Also included are antibody-drug conjugates.

A therapeutic agent may be a T-cell checkpoint inhibitor. In one embodiment, the checkpoint inhibitor is an inhibitory antibody (e.g., a monospecific antibody such as a monoclonal antibody). The antibody may be, e.g., humanized or fully human. In some embodiments, the checkpoint inhibitor is a fusion protein, e.g., an Fc-receptor fusion protein. In some embodiments, the checkpoint inhibitor is an agent, such as an antibody, that interacts with a checkpoint protein. In some embodiments, the checkpoint inhibitor is an agent, such as an antibody, that interacts with the ligand of a checkpoint protein.

In some embodiments, the checkpoint inhibitor is an inhibitor (e.g., an inhibitory antibody or small molecule inhibitor) of CTLA-4 (e.g., an anti-CTLA-4 antibody or fusion a protein). In some embodiments, the checkpoint inhibitor is an inhibitor or antagonist (e.g., an inhibitory antibody or small molecule inhibitor) of PD-1. In some embodiments, the checkpoint inhibitor is an inhibitor or antagonist (e.g., an inhibitory antibody or small molecule inhibitor) of PD-L1. In some embodiments, the checkpoint inhibitor is an inhibitor or antagonist (e.g., an inhibitory antibody or Fc fusion or small molecule inhibitor) of PD-L2 (e.g., a PD-L2/Ig fusion protein). In some embodiments, the checkpoint inhibitor is an inhibitor or antagonist (e.g., an inhibitory antibody or small molecule inhibitor) of B7-H3, B7-H4, BTLA, HVEM, TIM3, GAL9, LAG3, VISTA, KIR, 2B4, CD160, CGEN-15049, CHK 1, CHK2, A2aR, B-7 family ligands, or a combination thereof. In some embodiments, the checkpoint inhibitor is pembrolizumab, nivolumab, PDR001 (NVS), REGN2810 (Sanofi/Regeneron), a PD-L1 antibody such as, e.g., avelumab, durvalumab, atezolizumab, pidilizumab, JNJ-63723283 (JNJ), BGB-A317 (BeiGene & Celgene) or a checkpoint inhibitor disclosed in Preusser, M. et al. (2015) Nat. Rev. Neurol., including, without limitation, ipilimumab, tremelimumab, nivolumab, pembrolizumab, AMP224, AMP514/MED10680, BMS936559, MED14736, MPDL3280A, MSB0010718C, BMS986016, IMP321, lirilumab, IPH2101, 1-7F9, and KW-6002.

A therapeutic agent may be an anti-TIGIT antibody, such as MBSA43, BMS-986207, MK-7684, COM902, AB154, MTIG7192A or OMP-313M32 (etigilimab).

A therapeutic agent may be an agent that treats cancer or symptoms associated therewith (e.g., a cytotoxic agent, non-peptide small molecules, or other compound useful in the treatment of cancer or symptoms associated therewith, collectively, an “anti-cancer agent”). Anti-cancer agents can be, e.g., chemotherapeutics or targeted therapy agents.

Anti-cancer agents include mitotic inhibitors, intercalating antibiotics, growth factor inhibitors, cell cycle inhibitors, enzymes, topoisomerase inhibitors, biological response modifiers, alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Further anti-cancer agents include leucovorin (LV), irenotecan, oxaliplatin, capecitabine, paclitaxel, and doxetaxel. In some embodiments, the one or more additional therapies includes two or more anti-cancer agents. The two or more anti-cancer agents can be used in a cocktail to be administered in combination or administered separately. Suitable dosing regimens of combination anti-cancer agents are known in the art and described in, for example, Saltz et al., Proc. Am. Soc. Clin. Oncol. 18:233a (1999), and Douillard et al., Lancet 355(9209):1041-1047 (2000).

Other non-limiting examples of anti-cancer agents include Gleevec® (Irnatinib Mesylate); Kyprolis® (carfilzomib); Velcade® (bortezomib); Casodex (bicalutamide); Iressa® (gefitinib); alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; sarcodictyin A; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, such as calicheamicin gammall and calicheamicin omegall (see, e.g., Agnew, Chem. Intl. Ed Engl. 33:183-186 (1994)); dynemicin such as dynemicin A; bisphosphonates such as clodronate; an esperamicin; neocarzinostatin chromophore and related chromoprotein enediyne antibiotic chromophores, aclacinomysins, actinomycin, authramycin, azaserine, bleomycins, cactinomycin, calicheamicin, carabicin, caminomycin, carminomycin, carzinophilin, chromomycins, dactinomycin, daunorubicin, detorubicin, 6-diazo-5-oxo-L-norleucine, adriamycin (doxorubicin), morpholino-doxorubicin, cyanomorpholino-doxorubicin, 2-pyrrolino-doxorubicin, deoxydoxorubicin, epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenishers such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone such as epothilone B; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes such as T-2 toxin, verracurin A, roridin A and anguidine; urethane; vindesine; dacarbazine; mannomustine; mitobronitol; mitolactol; pipobroman; gacytosine; arabinoside (“Ara-C”); cyclophosphamide; thiotepa; taxoids, e.g., Taxol® (paclitaxel), Abraxane® (cremophor-free, albumin-engineered nanoparticle formulation of paclitaxel), and Taxotere® (doxetaxel); chloranbucil; tamoxifen (Nolvadex™); raloxifene; aromatase inhibiting 4(5)-imidazoles; 4-hydroxytamoxifen; trioxifene; keoxifene; LY 117018; onapristone; toremifene (Fareston®); flutamide, nilutamide, bicalutamide, leuprolide, goserelin; chlorambucil; Gemzar® gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; Navelbine® (vinorelbine); novantrone; teniposide; edatrexate; daunomycin; aminopterin; ibandronate; irinotecan (e.g., CPT-11); topoisomerase inhibitor RFS 2000; difluoromethylornithine (DMFO); retinoids such as retinoic acid; esperamicins; capecitabine (e.g., Xeloda®); and pharmaceutically acceptable salts of any of the above.

Additional non-limiting examples of anti-cancer agents include trastuzumab (Herceptin®), bevacizumab (Avastin®), cetuximab (Erbitux®), rituximab (Rituxan®), Taxol®, Arimidex®, ABVD, avicine, abagovomab, acridine carboxamide, adecatumumab, 17-N-allylamino-17-demethoxygeldanamycin, alpharadin, alvocidib, 3-aminopyridine-2-carboxaldehyde thiosemicarbazone, amonafide, anthracenedione, anti-CD22 immunotoxins, antineoplastics (e.g., cell-cycle nonspecific antineoplastic agents, and other antineoplastics described herein), antitumorigenic herbs, apaziquone, atiprimod, azathioprine, belotecan, bendamustine, BIBW 2992, biricodar, brostallicin, bryostatin, buthionine sulfoximine, CBV (chemotherapy), calyculin, dichloroacetic acid, discodermolide, elsamitrucin, enocitabine, eribulin, exatecan, exisulind, ferruginol, forodesine, fosfestrol, ICE chemotherapy regimen, IT-101, imexon, imiquimod, indolocarbazole, irofulven, laniquidar, larotaxel, lenalidomide, lucanthone, lurtotecan, mafosfamide, mitozolomide, nafoxidine, nedaplatin, olaparib, ortataxel, PAC-1, pawpaw, pixantrone, proteasome inhibitors, rebeccamycin, resiquimod, rubitecan, SN-38, salinosporamide A, sapacitabine, Stanford V, swainsonine, talaporfin, tariquidar, tegafur-uracil, temodar, tesetaxel, triplatin tetranitrate, tris(2-chloroethyl)amine, troxacitabine, uramustine, vadimezan, vinflunine, ZD6126, and zosuquidar.

Further non-limiting examples of anti-cancer agents include natural products such as vinca alkaloids (e.g., vinblastine, vincristine, and vinorelbine), epidipodophyllotoxins (e.g., etoposide and teniposide), antibiotics (e.g., dactinomycin (actinomycin D), daunorubicin, and idarubicin), anthracyclines, mitoxantrone, bleomycins, plicamycin (mithramycin), mitomycin, enzymes (e.g., L-asparaginase which systemically metabolizes L-asparagine and deprives cells which do not have the capacity to synthesize their own asparagine), antiplatelet agents, antiproliferative/antimitotic alkylating agents such as nitrogen mustards (e.g., mechlorethamine, cyclophosphamide and analogs, melphalan, and chlorambucil), ethylenimines and methylmelamines (e.g., hexaamethylmelaamine and thiotepa), CDK inhibitors (e.g., a CDK4/6 inhibitor such as abemaciclib, ribociclib, palbociclib; seliciclib, UCN-01, P1446A-05, PD-0332991, dinaciclib, P27-00, AT-7519, RGB286638, and SCH727965), alkyl sulfonates (e.g., busulfan), nitrosoureas (e.g., carmustine (BCNU) and analogs, and streptozocin), trazenes-dacarbazinine (DTIC), antiproliferative/antimitotic antimetabolites such as folic acid analogs, pyrimidine analogs (e.g., fluorouracil, floxuridine, and cytarabine), purine analogs and related inhibitors (e.g., mercaptopurine, thioguanine, pentostatin, and 2-chlorodeoxyadenosine), aromatase inhibitors (e.g., anastrozole, exemestane, and letrozole), and platinum coordination complexes (e.g., cisplatin and carboplatin), procarbazine, hydroxyurea, mitotane, aminoglutethimide, histone deacetylase (HDAC) inhibitors (e.g., trichostatin, sodium butyrate, apicidan, suberoyl anilide hydroamic acid, vorinostat, LBH 589, romidepsin, ACY-1215, and panobinostat), mTOR inhibitors (e.g., vistusertib, temsirolimus, everolimus, sapanasertib, ridaforolimus, and sirolimus), KSP(Eg5) inhibitors (e.g., Array 520), DNA binding agents (e.g., Zalypsis®), PI3K inhibitors such as PI3K delta inhibitor (e.g., GS-1101 and TGR-1202), PI3K delta and gamma inhibitor (e.g., CAL-130), copanlisib, alpelisib and idelalisib; multi-kinase inhibitor (e.g., TGO2 and sorafenib), hormones (e.g., estrogen) and hormone agonists such as leutinizing hormone releasing hormone (LHRH) agonists (e.g., goserelin, leuprolide and triptorelin), BAFF-neutralizing antibody (e.g., LY2127399), IKK inhibitors, p38MAPK inhibitors, anti-IL-6 (e.g., CNT0328), telomerase inhibitors (e.g., GRN 163L), aurora kinase inhibitors (e.g., MLN8237), cell surface monoclonal antibodies (e.g., anti-CD38 (HUMAX-CD38), anti-CSI (e.g., elotuzumab), HSP90 inhibitors (e.g., 17 AAG and KOS 953), PI3K/Akt inhibitors (e.g., perifosine), Akt inhibitors (e.g., GSK-2141795), PKC inhibitors (e.g., enzastaurin), FTIs (e.g., Zarnestra™), anti-CD138 (e.g., BT062), Torcl/2 specific kinase inhibitors (e.g., INK128), ER/UPR targeting agents (e.g., MKC-3946), cFMS inhibitors (e.g., ARRY-382), JAK1/2 inhibitors (e.g., CYT387), PARP inhibitors (e.g., olaparib and veliparib (ABT-888)), and BCL-2 antagonists.

In some embodiments, an anti-cancer agent is selected from mechlorethamine, camptothecin, ifosfamide, tamoxifen, raloxifene, gemcitabine, Navelbine®, sorafenib, or any analog or derivative variant of the foregoing.

In some embodiments, an anti-cancer agent is an ALK inhibitor. Non-limiting examples of ALK inhibitors include ceritinib, TAE-684 (NVP-TAE694), PF02341066 (crizotinib or 1066), alectinib; brigatinib; entrectinib; ensartinib (X-396); lorlatinib; ASP3026; CEP-37440; 4SC-203; TL-398; PLB1003; TSR-011; CT-707; TPX-0005, and AP26113. Additional examples of ALK kinase inhibitors are described in examples 3-39 of WO05016894.

In some embodiments, an anti-cancer agent is an inhibitor of a member downstream of a Receptor Tyrosine Kinase (RTK)/Growth Factor Receptor (e.g., a SHP2 inhibitor (e.g., SHP099, TNO155, RMC-4550, RMC-4630, JAB-3068, JAB-3312, RLY-1971, ERAS-601, SH3809, PF-07284892, or BBP-398 or any other SHP2 inhibitor known in the art), a SOS1 inhibitor (e.g., RMC-5845, BI-1701963, BI-3406, SDR5, or BAY-293 or any other SHP2 inhibitor known in the art), a RAS inhibitor (e.g., BI-2852 or any RAS inhibitor known in the art), a RAS degrader, a Raf inhibitor, a MEK inhibitor, an ERK inhibitor, a PI3K inhibitor, a PTEN inhibitor, an AKT inhibitor, or an mTOR inhibitor (e.g., mTORC1 inhibitor or mTORC2 inhibitor). In some embodiments, the anti-cancer agent is JAB-3312.

In some embodiments, a therapeutic agent that may be combined with a compound of the present disclosure is an inhibitor of the MAP kinase (MAPK) pathway (or “MAPK inhibitor”). MAPK inhibitors include, but are not limited to, one or more MAPK inhibitor described in Cancers (Basel) 2015 September; 7(3): 1758-1784. For example, the MAPK inhibitor may be selected from one or more of trametinib, binimetinib, selumetinib, cobimetinib, LErafAON (NeoPharm), ISIS 5132; vemurafenib, pimasertib, TAK733, R04987655 (CH4987655); CI-1040; PD-0325901; CH5126766; MAP855; AZD6244; refametinib (RDEA 119/BAY 86-9766); GDC-0973/XL581; AZD8330 (ARRY-424704/ARRY-704); R05126766 (Roche, described in PLoS One. 2014 Nov. 25; 9(11)); and GSK1120212 (or JTP-74057, described in Clin Cancer Res. 2011 Mar. 1; 17(5):989-1000). The MAPK inhibitor may be PLX8394, LXH254, GDC-5573, or LY3009120.

In some embodiments, an anti-cancer agent is a disrupter or inhibitor of the RAS-RAF-ERK or PI3K-AKT-TOR or PI3K-AKT signaling pathways. The PI3K/AKT inhibitor may include, but is not limited to, one or more PI3K/AKT inhibitor described in Cancers (Basel) 2015 September; 7(3): 1758-1784. For example, the PI3K/AKT inhibitor may be selected from one or more of NVP-BEZ235; BGT226; XL765/SAR245409; SF1126; GDC-0980; PI-103; PF-04691502; PKI-587; GSK2126458.

In some embodiments, an anti-cancer agent is a PD-1 or PD-L1 antagonist.

In some embodiments, additional therapeutic agents include ALK inhibitors, HER2 inhibitors, EGFR inhibitors, IGF-1R inhibitors, MEK inhibitors, PI3K inhibitors, AKT inhibitors, TOR inhibitors, MCL-1 inhibitors, BCL-2 inhibitors, SHP2 inhibitors, proteasome inhibitors, and immune therapies, such as an immune checkpoint inhibitor.

MEK inhibitors include, but are not limited to, pimasertib, selumetinib, cobimetinib (Cotellic®), trametinib (Mekinist®), and binimetinib (Mektovi®). In some embodiments, a MEK inhibitor targets a MEK mutation that is a Class I MEK1 mutation selected from D67N; P124L; P124S; and L177V. In some embodiments, the MEK mutation is a Class II MEK1 mutation selected from ΔE51-Q58; ΔF53-Q58; E203K; L177M; C121S; F53L; K57E; Q56P; and K57N.

PI3K inhibitors include, but are not limited to, wortmannin; 17-hydroxywortmannin analogs described in WO06/044453; 4-[2-(1H-Indazol-4-yl)-6-[[4-(methylsulfonyl)piperazin-1-yl]methyl]thieno[3,2-d]pyrimidin-4-yl]morpholine (also known as pictilisib or GDC-0941 and described in WO09/036082 and WO09/055730); 2-methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in WO06/122806); (S)-1-(4-((2-(2-aminopyrimidin-5-yl)-7-methyl-4-morpholinothieno[3,2-d]pyrimidin-6-yl)methyl)piperazin-1-yl)-2-hydroxypropan-1-one (described in WO08/070740); LY294002 (2-(4-morpholinyl)-8-phenyl-4H-1-benzopyran-4-one (available from Axon Medchem); PI 103 hydrochloride (3-[4-(4-morpholinylpyrido-[3′,2′:4,5]furo[3,2-d]pyrimidin-2-yl]phenol hydrochloride (available from Axon Medchem); PIK 75 (2-methyl-5-nitro-2-[(6-bromoimidazo[1,2-a]pyridin-3-yl)methylene]-1-methylhydrazide-benzenesulfonic acid, monohydrochloride) (available from Axon Medchem); PIK 90 (N-(7,8-dimethoxy-2,3-dihydro-imidazo[1,2-c]quinazolin-5-yl)-nicotinamide (available from Axon Medchem); AS-252424 (5-[1-[5-(4-fluoro-2-hydroxy-phenyl)-furan-2-yl]-meth-(Z)-ylidene]-thiazolidine-2,4-dione (available from Axon Medchem); TGX-221 (7-methyl-2-(4-morpholinyl)-9-[1-(phenylamino)ethyl]-4H-pyrido-[1,2-a]pyrindin-4-one (available from Axon Medchem); XL-765; and XL-147. Other PI3K inhibitors include demethoxyviridin, perifosine, CAL101, PX-866, BEZ235, SF1126, INK1117, IPI-145, BKM120, XL147, XL765, Palomid 529, GSK1059615, ZSTK474, PWT33597, IC87114, TGI 00-115, CAL263, PI-103, GNE-477, CUDC-907, and AEZS-136.

AKT inhibitors include, but are not limited to, Akt-1-1 (inhibits Aktl) (Barnett et al., Biochem. J. 2005, 385(Pt. 2): 399-408); Akt-1-1,2 (inhibits Akl and 2) (Barnett et al., Biochem. J. 2005, 385(Pt. 2): 399-408); API-59CJ-Ome (e.g., Jin et al., Br. J. Cancer 2004, 91:1808-12); 1-H-imidazo[4,5-c]pyridinyl compounds (e.g., WO 05/011700); indole-3-carbinol and derivatives thereof (e.g., U.S. Pat. No. 6,656,963; Sarkar and Li J Nutr. 2004, 134(12 Suppl):3493S-3498S); perifosine (e.g., interferes with Akt membrane localization; Dasmahapatra et al. Clin. Cancer Res. 2004, 10(15):5242-52); phosphatidylinositol ether lipid analogues (e.g., Gills and Dennis Expert. Opin. Investig. Drugs 2004, 13:787-97); and triciribine (TCN or API-2 or NCI identifier: NSC 154020; Yang et al., Cancer Res. 2004, 64:4394-9).

mTOR inhibitors include, but are not limited to, ATP-competitive mTORC1/mTORC2 inhibitors, e.g., PI-103, PP242, PP30; Torin 1; FKBP12 enhancers; 4H-1-benzopyran-4-one derivatives; and rapamycin (also known as sirolimus) and derivatives thereof, including: temsirolimus (Torisel®); everolimus (Afinitor®; WO94/09010); sapanasertib, ridaforolimus (also known as deforolimus or AP23573); rapalogs, e.g., as disclosed in WO98/02441 and WO01/14387, e.g. AP23464 and AP23841; 40-(2-hydroxyethyl)rapamycin; 40-[3-hydroxy(hydroxymethyl)methylpropanoate]-rapamycin (also known as CC1779); 40-epi-(tetrazolyt)-rapamycin (also called ABT578); 32-deoxorapamycin; 16-pentynyloxy-32(S)-dihydrorapanycin; derivatives disclosed in WO05/005434; derivatives disclosed in U.S. Pat. Nos. 5,258,389, 5,118,677, 5,118,678, 5,100,883, 5,151,413, 5,120,842, and 5,256,790, and in WO94/090101, WO92/05179, WO93/111130, WO94/02136, WO94/02485, WO95/14023, WO94/02136, WO95/16691, WO96/41807, WO96/41807, and WO2018204416; and phosphorus-containing rapamycin derivatives (e.g., WO05/016252). In some embodiments, the mTOR inhibitor is a bisteric inhibitor (see, e.g., WO2018204416, WO2019212990 and WO2019212991), such as RMC-5552.

BRAF inhibitors that may be used in combination with compounds of the disclosure include, for example, vemurafenib, dabrafenib, and encorafenib. A BRAF may comprise a Class 3 BRAF mutation. In some embodiments, the Class 3 BRAF mutation is selected from one or more of the following amino acid substitutions in human BRAF: D287H; P367R; V459L; G466V; G466E; G466A; S467L; G469E; N581S; N581I; D594N; D594G; D594A; D594H; F595L; G596D; G596R and A762E.

MCL-1 inhibitors include, but are not limited to, AMG-176, MIK665, and S63845. The myeloid cell leukemia-1 (MCL-1) protein is one of the key anti-apoptotic members of the B-cell lymphoma-2 (BCL-2) protein family. Over-expression of MCL-1 has been closely related to tumor progression as well as to resistance, not only to traditional chemotherapies but also to targeted therapeutics including BCL-2 inhibitors such as ABT-263.

In some embodiments, the additional therapeutic agent is selected from the group consisting of a MEK inhibitor, a HER2 inhibitor, a SHP2 inhibitor, a CDK4/6 inhibitor, an mTOR inhibitor, a SOS1 inhibitor, and a PD-L1 inhibitor. In some embodiments, the additional therapeutic agent is selected from the group consisting of a MEK inhibitor, a SHP2 inhibitor, and a PD-L1 inhibitor. See, e.g., Hallin et al., Cancer Discovery, DOI: 10.1158/2159-8290 (Oct. 28, 2019) and Canon et al., Nature, 575:217 (2019). In some embodiments, a Ras inhibitor of the present disclosure is used in combination with a MEK inhibitor and a SOS1 inhibitor. In some embodiments, a Ras inhibitor of the present disclosure is used in combination with a PD-L1 inhibitor and a SOS1 inhibitor. In some embodiments, a Ras inhibitor of the present disclosure is used in combination with a PD-L1 inhibitor and a SHP2 inhibitor. In some embodiments, a Ras inhibitor of the present disclosure is used in combination with a MEK inhibitor and a SHP2 inhibitor. In some embodiments, the cancer is colorectal cancer, and the treatment comprises administration of a Ras inhibitor of the present disclosure in combination with a second or third therapeutic agent.

Proteasome inhibitors include, but are not limited to, carfilzomib (Kyprolis®), bortezomib (Velcade®), and oprozomib.

Immune therapies include, but are not limited to, monoclonal antibodies, immunomodulatory imides (IMiDs), GITR agonists, genetically engineered T-cells (e.g., CAR-T cells), bispecific antibodies (e.g., BiTEs), and anti-PD-1, anti-PD-L1, anti-CTLA4, anti-LAGI, and anti-OX40 agents).

Immunomodulatory agents (IMiDs) are a class of immunomodulatory drugs (drugs that adjust immune responses) containing an imide group. The IMiD class includes thalidomide and its analogues (lenalidomide, pomalidomide, and apremilast).

Exemplary anti-PD-1 antibodies and methods for their use are described by Goldberg et al., Blood 2007, 110(1):186-192; Thompson et al., Clin. Cancer Res. 2007, 13(6):1757-1761; and WO06/121168 A1), as well as described elsewhere herein.

GITR agonists include, but are not limited to, GITR fusion proteins and anti-GITR antibodies (e.g., bivalent anti-GITR antibodies), such as, a GITR fusion protein described in U.S. Pat. Nos. 6,111,090, 8,586,023, WO2010/003118 and WO2011/090754; or an anti-GITR antibody described, e.g., in U.S. Pat. No. 7,025,962, EP 1947183, U.S. Pat. Nos. 7,812,135, 8,388,967, 8,591,886, 7,618,632, EP 1866339, and WO2011/028683, WO2013/039954, WO05/007190, WO07/133822, WO05/055808, WO99/40196, WO01/03720, WO99/20758, WO06/083289, WO05/115451, and WO2011/051726.

Another example of a therapeutic agent that may be used in combination with the compounds of the disclosure is an anti-angiogenic agent. Anti-angiogenic agents are inclusive of, but not limited to, in vitro synthetically prepared chemical compositions, antibodies, antigen binding regions, radionuclides, and combinations and conjugates thereof. An anti-angiogenic agent can be an agonist, antagonist, allosteric modulator, toxin or, more generally, may act to inhibit or stimulate its target (e.g., receptor or enzyme activation or inhibition), and thereby promote cell death or arrest cell growth. In some embodiments, the one or more additional therapies include an anti-angiogenic agent.

Anti-angiogenic agents can be MMP-2 (matrix-metalloproteinase 2) inhibitors, MMP-9 (matrix-metalloprotienase 9) inhibitors, and COX-II (cyclooxygenase 11) inhibitors. Non-limiting examples of anti-angiogenic agents include rapamycin, temsirolimus (CCI-779), everolimus (RAD001), sorafenib, sunitinib, and bevacizumab. Examples of useful COX-II inhibitors include alecoxib, valdecoxib, and rofecoxib.

Examples of useful matrix metalloproteinase inhibitors are described in WO96/33172, WO96/27583, WO98/07697, WO98/03516, WO98/34918, WO98/34915, WO98/33768, WO98/30566, WO90/05719, WO99/52910, WO99/52889, WO99/29667, WO99007675, EP0606046, EP0780386, EP1786785, EP1181017, EP0818442, EP1004578, and US20090012085, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 or AMP-9 relative to the other matrix-metalloproteinases (i.e., MAP-1, MMP-3, MMP-4, MMP-5, MMP-6, MMP-7, MMP-8, MMP-10, MMP-11, MMP-12, and MMP-13). Some specific examples of MMP inhibitors are AG-3340, RO 32-3555, and RS 13-0830.

Further exemplary anti-angiogenic agents include KDR (kinase domain receptor) inhibitory agents (e.g., antibodies and antigen binding regions that specifically bind to the kinase domain receptor), anti-VEGF agents (e.g., antibodies or antigen binding regions that specifically bind VEGF (e.g., bevacizumab), or soluble VEGF receptors or a ligand binding region thereof) such as VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as Vectibix® (panitumumab), erlotinib (Tarceva®), anti-Angl and anti-Ang2 agents (e.g., antibodies or antigen binding regions specifically binding thereto or to their receptors, e.g., Tie2/Tek), and anti-Tie2 kinase inhibitory agents (e.g., antibodies or antigen binding regions that specifically bind thereto). Other anti-angiogenic agents include Campath, IL-8, B-FGF, Tek antagonists (US2003/0162712; U.S. Pat. No. 6,413,932), anti-TWEAK agents (e.g., specifically binding antibodies or antigen binding regions, or soluble TWEAK receptor antagonists; see U.S. Pat. No. 6,727,225), ADAM distintegrin domain to antagonize the binding of integrin to its ligands (US 2002/0042368), specifically binding anti-eph receptor or anti-ephrin antibodies or antigen binding regions (U.S. Pat. Nos. 5,981,245; 5,728,813; 5,969,110; 6,596,852; 6,232,447; 6,057,124 and patent family members thereof), and anti-PDGF-BB antagonists (e.g., specifically binding antibodies or antigen binding regions) as well as antibodies or antigen binding regions specifically binding to PDGF-BB ligands, and PDGFR kinase inhibitory agents (e.g., antibodies or antigen binding regions that specifically bind thereto). Additional anti-angiogenic agents include: SD-7784 (Pfizer, USA); cilengitide (Merck KGaA, Germany, EPO 0770622); pegaptanib octasodium, (Gilead Sciences, USA); Alphastatin, (BioActa, UK); M-PGA, (Celgene, USA, U.S. Pat. No. 5,712,291); ilomastat, (Arriva, USA, U.S. Pat. No. 5,892,112); emaxanib, (Pfizer, USA, U.S. Pat. No. 5,792,783); vatalanib, (Novartis, Switzerland); 2-methoxyestradiol (EntreMed, USA); TLC ELL-12 (Elan, Ireland); anecortave acetate (Alcon, USA); alpha-D148 Mab (Amgen, USA); CEP-7055 (Cephalon, USA); anti-Vn Mab (Crucell, Netherlands), DACantiangiogenic (ConjuChem, Canada); Angiocidin (InKine Pharmaceutical, USA); KM-2550 (Kyowa Hakko, Japan); SU-0879 (Pfizer, USA); CGP-79787 (Novartis, Switzerland, EP 0970070); ARGENT technology (Ariad, USA); YIGSR-Stealth (Johnson & Johnson, USA); fibrinogen-E fragment (BioActa, UK); angiogenic inhibitor (Trigen, UK); TBC-1635 (Encysive Pharmaceuticals, USA); SC-236 (Pfizer, USA); ABT-567 (Abbott, USA); Metastatin (EntreMed, USA); maspin (Sosei, Japan); 2-methoxyestradiol (Oncology Sciences Corporation, USA); ER-68203-00 (IV AX, USA); BeneFin (Lane Labs, USA); Tz-93 (Tsumura, Japan); TAN-1120 (Takeda, Japan); FR-111142 (Fujisawa, Japan, JP 02233610); platelet factor 4 (RepliGen, USA, EP 407122); vascular endothelial growth factor antagonist (Borean, Denmark); bevacizumab (pINN) (Genentech, USA); angiogenic inhibitors (SUGEN, USA); XL 784 (Exelixis, USA); XL 647 (Exelixis, USA); MAb, alpha5beta3 integrin, second generation (Applied Molecular Evolution, USA and MedImmune, USA); enzastaurin hydrochloride (Lilly, USA); CEP 7055 (Cephalon, USA and Sanofi-Synthelabo, France); BC 1 (Genoa Institute of Cancer Research, Italy); rBPI 21 and BPI-derived antiangiogenic (XOMA, USA); PI 88 (Progen, Australia); cilengitide (Merck KGaA, German; Munich Technical University, Germany, Scripps Clinic and Research Foundation, USA); AVE 8062 (Ajinomoto, Japan); AS 1404 (Cancer Research Laboratory, New Zealand); SG 292, (Telios, USA); Endostatin (Boston Childrens Hospital, USA); ATN 161 (Attenuon, USA); 2-methoxyestradiol (Boston Childrens Hospital, USA); ZD 6474, (AstraZeneca, UK); ZD 6126, (Angiogene Pharmaceuticals, UK); PPI 2458, (Praecis, USA); AZD 9935, (AstraZeneca, UK); AZD 2171, (AstraZeneca, UK); vatalanib (pINN), (Novartis, Switzerland and Schering AG, Germany); tissue factor pathway inhibitors, (EntreMed, USA); pegaptanib (Pinn), (Gilead Sciences, USA); xanthorrhizol, (Yonsei University, South Korea); vaccine, gene-based, VEGF-2, (Scripps Clinic and Research Foundation, USA); SPV5.2, (Supratek, Canada); SDX 103, (University of California at San Diego, USA); PX 478, (ProIX, USA); METASTATIN, (EntreMed, USA); troponin I, (Harvard University, USA); SU 6668, (SUGEN, USA); OXI 4503, (OXiGENE, USA); o-guanidines, (Dimensional Pharmaceuticals, USA); motuporamine C, (British Columbia University, Canada); CDP 791, (Celltech Group, UK); atiprimod (pINN), (GlaxoSmithKline, UK); E 7820, (Eisai, Japan); CYC 381, (Harvard University, USA); AE 941, (Aeterna, Canada); vaccine, angiogenic, (EntreMed, USA); urokinase plasminogen activator inhibitor, (Dendreon, USA); oglufanide (pINN), (Melmotte, USA); HIF-lalfa inhibitors, (Xenova, UK); CEP 5214, (Cephalon, USA); BAY RES 2622, (Bayer, Germany); Angiocidin, (InKine, USA); A6, (Angstrom, USA); KR 31372, (Korea Research Institute of Chemical Technology, South Korea); GW 2286, (GlaxoSmithKline, UK); EHT 0101, (ExonHit, France); CP 868596, (Pfizer, USA); CP 564959, (OSI, USA); CP 547632, (Pfizer, USA); 786034, (GlaxoSmithKline, UK); KRN 633, (Kirin Brewery, Japan); drug delivery system, intraocular, 2-methoxyestradiol; anginex (Maastricht University, Netherlands, and Minnesota University, USA); ABT 510 (Abbott, USA); AAL 993 (Novartis, Switzerland); VEGI (ProteomTech, USA); tumor necrosis factor-alpha inhibitors; SU 11248 (Pfizer, USA and SUGEN USA); ABT 518, (Abbott, USA); YH16 (Yantai Rongchang, China); S-3APG (Boston Childrens Hospital, USA and EntreMed, USA); MAb, KDR (ImClone Systems, USA); MAb, alpha5 beta (Protein Design, USA); KDR kinase inhibitor (Celltech Group, UK, and Johnson & Johnson, USA); GFB 116 (South Florida University, USA and Yale University, USA); CS 706 (Sankyo, Japan); combretastatin A4 prodrug (Arizona State University, USA); chondroitinase AC (IBEX, Canada); BAY RES 2690 (Bayer, Germany); AGM 1470 (Harvard University, USA, Takeda, Japan, and TAP, USA); AG 13925 (Agouron, USA); Tetrathiomolybdate (University of Michigan, USA); GCS 100 (Wayne State University, USA) CV 247 (Ivy Medical, UK); CKD 732 (Chong Kun Dang, South Korea); irsogladine, (Nippon Shinyaku, Japan); RG 13577 (Aventis, France); WX 360 (Wilex, Germany); squalamine, (Genaera, USA); RPI 4610 (Sirna, USA); heparanase inhibitors (InSight, Israel); KL 3106 (Kolon, South Korea); Honokiol (Emory University, USA); ZK CDK (Schering AG, Germany); ZK Angio (Schering AG, Germany); ZK 229561 (Novartis, Switzerland, and Schering AG, Germany); XMP 300 (XOMA, USA); VGA 1102 (Taisho, Japan); VE-cadherin-2 antagonists(ImClone Systems, USA); Vasostatin (National Institutes of Health, USA); Flk-1 (ImClone Systems, USA); TZ 93 (Tsumura, Japan); TumStatin (Beth Israel Hospital, USA); truncated soluble FLT 1 (vascular endothelial growth factor receptor 1) (Merck & Co, USA); Tie-2 ligands (Regeneron, USA); and thrombospondin 1 inhibitor (Allegheny Health, Education and Research Foundation, USA).

Further examples of therapeutic agents that may be used in combination with compounds of the disclosure include agents (e.g., antibodies, antigen binding regions, or soluble receptors) that specifically bind and inhibit the activity of growth factors, such as antagonists of hepatocyte growth factor (HGF, also known as Scatter Factor), and antibodies or antigen binding regions that specifically bind its receptor, c-Met.

Another example of a therapeutic agent that may be used in combination with compounds of the disclosure is an autophagy inhibitor. Autophagy inhibitors include, but are not limited to chloroquine, 3-methyladenine, hydroxychloroquine (Plaquenil™), bafilomycin A1, 5-amino-4-imidazole carboxamide riboside (AICAR), okadaic acid, autophagy-suppressive algal toxins which inhibit protein phosphatases of type 2A or type 1, analogues of cAMP, and drugs which elevate cAMP levels such as adenosine, LY204002, N₆-mercaptopurine riboside, and vinblastine. In addition, antisense or siRNA that inhibits expression of proteins including but not limited to ATG5 (which are implicated in autophagy), may also be used. In some embodiments, the one or more additional therapies include an autophagy inhibitor.

Another example of a therapeutic agent that may be used in combination with compounds of the disclosure is an anti-neoplastic agent. In some embodiments, the one or more additional therapies include an anti-neoplastic agent. Non-limiting examples of anti-neoplastic agents include acemannan, aclarubicin, aldesleukin, alemtuzumab, alitretinoin, altretamine, amifostine, aminolevulinic acid, amrubicin, amsacrine, anagrelide, anastrozole, ancer, ancestim, arglabin, arsenic trioxide, BAM-002 (Novelos), bexarotene, bicalutamide, broxuridine, capecitabine, celmoleukin, cetrorelix, cladribine, clotrimazole, cytarabine ocfosfate, DA 3030 (Dong-A), daclizumab, denileukin diftitox, deslorelin, dexrazoxane, dilazep, docetaxel, docosanol, doxercalciferol, doxifluridine, doxorubicin, bromocriptine, carmustine, cytarabine, fluorouracil, HIT diclofenac, interferon alfa, daunorubicin, doxorubicin, tretinoin, edelfosine, edrecolomab, eflornithine, emitefur, epirubicin, epoetin beta, etoposide phosphate, exemestane, exisulind, fadrozole, filgrastim, finasteride, fludarabine phosphate, formestane, fotemustine, gallium nitrate, gemcitabine, gemtuzumab zogamicin, gimeracil/oteracil/tegafur combination, glycopine, goserelin, heptaplatin, human chorionic gonadotropin, human fetal alpha fetoprotein, ibandronic acid, idarubicin, (imiquimod, interferon alfa, interferon alfa, natural, interferon alfa-2, interferon alfa-2a, interferon alfa-2b, interferon alfa-NI, interferon alfa-n3, interferon alfacon-1, interferon alpha, natural, interferon beta, interferon beta-la, interferon beta-lb, interferon gamma, natural interferon gamma-la, interferon gamma-Ib, interleukin-1 beta, iobenguane, irinotecan, irsogladine, Ianreotide, LC 9018 (Yakult), leflunomide, lenograstim, lentinan sulfate, letrozole, leukocyte alpha interferon, leuprorelin, levamisole+fluorouracil, liarozole, lobaplatin, lonidamine, lovastatin, masoprocol, melarsoprol, metoclopramide, mifepristone, miltefosine, mirimostim, mismatched double stranded RNA, mitoguazone, mitolactol, mitoxantrone, molgramostim, nafarelin, naloxone+pentazocine, nartograstim, nedaplatin, nilutamide, noscapine, novel erythropoiesis stimulating protein, NSC 631570 octreotide, oprelvekin, osaterone, oxaliplatin, paclitaxel, pamidronic acid, pegaspargase, peginterferon alfa-2b, pentosan polysulfate sodium, pentostatin, picibanil, pirarubicin, rabbit antithymocyte polyclonal antibody, polyethylene glycol interferon alfa-2a, porfimer sodium, raloxifene, raltitrexed, rasburiembodiment, rhenium Re 186 etidronate, RII retinamide, rituximab, romurtide, samarium (153 Sm) lexidronam, sargramostim, sizofiran, sobuzoxane, sonermin, strontium-89 chloride, suramin, tasonermin, tazarotene, tegafur, temoporfin, temozolomide, teniposide, tetrachlorodecaoxide, thalidomide, thymalfasin, thyrotropin alfa, topotecan, toremifene, tositumomab-iodine 131, trastuzumab, treosulfan, tretinoin, trilostane, trimetrexate, triptorelin, tumor necrosis factor alpha, natural, ubenimex, bladder cancer vaccine, Maruyama vaccine, melanoma lysate vaccine, valrubicin, verteporfin, vinorelbine, virulizin, zinostatin stimalamer, or zoledronic acid; abarelix; AE 941 (Aeterna), ambamustine, antisense oligonucleotide, bcl-2 (Genta), APC 8015 (Dendreon), decitabine, dexaminoglutethimide, diaziquone, EL 532 (Elan), EM 800 (Endorecherche), eniluracil, etanidazole, fenretinide, filgrastim SD01 (Amgen), fulvestrant, galocitabine, gastrin 17 immunogen, HLA-B7 gene therapy (Vical), granulocyte macrophage colony stimulating factor, histamine dihydrochloride, ibritumomab tiuxetan, ilomastat, IM 862 (Cytran), interleukin-2, iproxifene, LDI 200 (Milkhaus), leridistim, lintuzumab, CA 125 MAb (Biomira), cancer MAb (Japan Pharmaceutical Development), HER-2 and Fc MAb (Medarex), idiotypic 105AD7 MAb (CRC Technology), idiotypic CEA MAb (Trilex), LYM-1-iodine 131 MAb (Techni clone), polymorphic epithelial mucin-yttrium 90 MAb (Antisoma), marimastat, menogaril, mitumomab, motexafin gadolinium, MX 6 (Galderma), nelarabine, nolatrexed, P 30 protein, pegvisomant, pemetrexed, porfiromycin, prinomastat, RL 0903 (Shire), rubitecan, satraplatin, sodium phenylacetate, sparfosic acid, SRL 172 (SR Pharma), SU 5416 (SUGEN), TA 077 (Tanabe), tetrathiomolybdate, thaliblastine, thrombopoietin, tin ethyl etiopurpurin, tirapazamine, cancer vaccine (Biomira), melanoma vaccine (New York University), melanoma vaccine (Sloan Kettering Institute), melanoma oncolysate vaccine (New York Medical College), viral melanoma cell lysates vaccine (Royal Newcastle Hospital), or valspodar.

Additional examples of therapeutic agents that may be used in combination with compounds of the disclosure include ipilimumab (Yervoy®); tremelimumab; galiximab; nivolumab, also known as BMS-936558 (Opdivo®); pembrolizumab (Keytruda®); avelumab (Bavencio®); AMP224; BMS-936559; MPDL3280A, also known as RG7446; MEDI-570; AMG557; MGA271; IMP321; BMS-663513; PF-05082566; CDX-1127; anti-OX40 (Providence Health Services); huMAbOX40L; atacicept; CP-870893; lucatumumab; dacetuzumab; muromonab-CD3; ipilumumab; MEDI4736 (Imfinzi®); MSB0010718C; AMP 224; adalimumab (Humira®); ado-trastuzumab emtansine (Kadcyla®); aflibercept (Eylea®); alemtuzumab (Campath®); basiliximab (Simulect®); belimumab (Benlysta®); basiliximab (Simulect®); belimumab (Benlysta®); brentuximab vedotin (Adcetris®); canakinumab (Ilaris®); certolizumab pegol (Cimzia®); daclizumab (Zenapax®); daratumumab (Darzalex®); denosumab (Prolia®); eculizumab (Soliris®); efalizumab (Raptiva®); gemtuzumab ozogamicin (Mylotarg®); golimumab (Simponi®); ibritumomab tiuxetan (Zevalin®); infliximab (Remicade®); motavizumab (Numax®); natalizumab (Tysabri®); obinutuzumab (Gazyva®); ofatumumab (Arzerra®); omalizumab (Xolair®); palivizumab (Synagis®); pertuzumab (Perjeta®); pertuzumab (Perjeta®); ranibizumab (Lucentis®); raxibacumab (Abthrax®); tocilizumab (Actemra®); tositumomab; tositumomab-i-131; tositumomab and tositumomab-i-131 (Bexxar®); ustekinumab (Stelara®); AMG 102; AMG 386; AMG 479; AMG 655; AMG 706; AMG 745; and AMG 951.

The compounds described herein can be used in combination with the agents disclosed herein or other suitable agents, depending on the condition being treated. Hence, in some embodiments the one or more compounds of the disclosure will be co-administered with other therapies as described herein.

When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described herein can be formulated together in the same dosage form and administered simultaneously. Alternatively, a compound of the disclosure and any of the therapies described herein can be simultaneously administered, wherein both the agents are present in separate formulations. In another alternative, a compound of the present disclosure can be administered and followed by any of the therapies described herein, or vice versa. In some embodiments of the separate administration protocol, a compound of the disclosure and any of the therapies described herein are administered a few minutes apart, or a few hours apart, or a few days apart.

In some embodiments of any of the methods described herein, the first therapy (e.g., a compound of the disclosure) and one or more additional therapies are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours, up to 24 hours, or up to 1-7, 1-14, 1-21 or 1-30 days before or after the one or more additional therapies.

The disclosure also features kits including (a) a pharmaceutical composition including an agent (e.g., a compound of the disclosure) described herein, and (b) a package insert with instructions to perform any of the methods described herein. In some embodiments, the kit includes (a) a pharmaceutical composition including an agent (e.g., a compound of the disclosure) described herein, (b) one or more additional therapies (e.g., non-drug treatment or therapeutic agent), and (c) a package insert with instructions to perform any of the methods described herein.

As one aspect of the present disclosure contemplates the treatment of the disease or symptoms associated therewith with a combination of pharmaceutically active compounds that may be administered separately, the disclosure further relates to combining separate pharmaceutical compositions in kit form.

The kit may comprise two separate pharmaceutical compositions: a compound of the present disclosure, and one or more additional therapies. The kit may comprise a container for containing the separate compositions such as a divided bottle or a divided foil packet. Additional examples of containers include syringes, boxes, and bags. In some embodiments, the kit may comprise directions for the use of the separate components. The kit form is particularly advantageous when the separate components are preferably administered in different dosage forms (e.g., oral and parenteral), are administered at different dosage intervals, or when titration of the individual components of the combination is desired by the prescribing health care professional.

### EMBODIMENTS

Embodiment 1: A method of treating cancer in a subject in need thereof, the method comprising administering to the subject a therapeutically effective amount of a RAS(ON) GTP hydrolysis-promoting compound, wherein the cancer does not comprise a RAS mutation at position 61.

Embodiment 2: A method of treating cancer in a subject in need thereof, the method comprising administering to the subject a therapeutically effective amount of a RAS(ON) GTP hydrolysis-promoting compound and a RAS(OFF) inhibitor, wherein the cancer does not comprise a RAS mutation at position 61.

Embodiment 3: A method of treating cancer in a subject in need thereof, the method comprising administering to the subject a therapeutically effective amount of a RAS(ON) GTP hydrolysis-promoting compound and a RTK inhibitor, wherein the cancer does not comprise a RAS mutation at position 61.

Embodiment 4: A method of treating cancer in a subject in need thereof, the method comprising administering to the subject a therapeutically effective amount of a RAS(ON) GTP hydrolysis-promoting compound and a SHP inhibitor (e.g., a SHP2 inhibitor), wherein the cancer does not comprise a RAS mutation at position 61.

Embodiment 5: A method of treating cancer in a subject in need thereof, the method comprising administering to the subject a therapeutically effective amount of a RAS(ON) GTP hydrolysis-promoting compound and a SOS1 inhibitor, wherein the cancer does not comprise a RAS mutation at position 61.

Embodiment 6: The method of any one of Embodiments 1 to 5, wherein the cancer comprises a RAS mutation.

Embodiment 7: The method of Embodiment 6, wherein the RAS mutation is a RAS amplification or the RAS mutation is at position 12 or 13.

Embodiment 8: The method of any one of Embodiments 1 to 7, wherein the cancer is pancreatic cancer, colorectal cancer, non-small cell lung cancer, gastric cancer, esophageal cancer, ovarian cancer, or uterine cancer.

Embodiment 9: The method of any one of Embodiments 1 to 8, wherein binding of the RAS(ON) GTP hydrolysis-promoting compound to RAS(ON) alters the position of glutamine 61 of RAS(ON), relative to the position in the absence of the RAS(ON) GTP hydrolysis-promoting compound, towards the gamma phosphate of GTP bound to the RAS(ON) protein, thereby increasing the GTP hydrolysis rate relative to the hydrolysis rate of RAS(ON) in the absence of the RAS(ON) GTP hydrolysis-promoting compound.

Embodiment 10: The method of any one of Embodiments 1 to 9, wherein the RAS(ON) GTP-hydrolysis promoting compound is a compound of Table 1 or a pharmaceutically acceptable salt thereof.

Embodiment 11: The method of any one of Embodiments 2 or 6-10, wherein the RAS(OFF) inhibitor is a KRAS(OFF) inhibitor.

Embodiment 12: The method of Embodiment 11, wherein the KRAS(OFF) inhibitor is a KRAS^{G12C}(OFF) inhibitor.

Embodiment 13: The method of Embodiment 12, wherein the KRAS^{G12C}(OFF) inhibitor is selected from the group consisting of AMG510 (sotorasib), MRTX849 (adagrasib), MRTX1257, GDC-6036 (divarasib), JDQ443 (opnurasib), ERAS-3490, LY3537982 (olomorasib), BI 1823911, BPI-421286, JAB-3312, JAB-21000, JAB-21822 (glecirasib), D-1553, D3S-001, HBI-2438, HS-10370, MK-1084, YL-15293, BBO—8520, FMC-376, GEC255, and GFH925 (1B1351).

Embodiment 14: The method of Embodiment 11, wherein the KRAS(OFF) inhibitor is a KRAS^{G12D}(OFF) inhibitor.

Embodiment 15: The method of Embodiment 14, wherein the KRAS^{G12D}(OFF) inhibitor is selected from the group consisting of MRTX1133, MRTX282, JAB-22000, ERAS-4, ERAS-5024, HRS-4642, BI-2852, ASP3082, TH-Z827, TH-7835, QTX-3046, GFH375 (VS-7375), INCB161734, and KD-8.

Embodiment 16: The method of Embodiment 11, wherein the KRAS(OFF) inhibitor is a KRAS^{G12V}(OFF) inhibitor.

Embodiment 17: The method of Embodiment 16, wherein the KRAS^{G12V}(OFF) inhibitor is JAB-23000.

Embodiment 18: The method of Embodiment 11, wherein the KRAS(OFF) inhibitor is a pan-RAS(OFF) inhibitor.

Embodiment 19: The method of Embodiment 18, wherein the pan-RAS(OFF) inhibitor is JAB-23400, JAB-23425, BI-2493, BI-2865, QTX-3034, QTX3544, ZG2001, BBO-a, BBO-B, or pan KRas-IN-1.

Embodiment 20: The method of any one of Embodiments 4 or 6-10, wherein the SHP2 inhibitor is selected from SHP099, TNO155, RMC-4550, RMC-4630, JAB-3068, JAB-3312, RLY-1971, ERAS-601, SH3809, PF-07284892, BBP-398, and any combination thereof.

Embodiment 21: The method of any one of Embodiment 5 to 10, wherein the SOS1 inhibitor is selected from RMC-5845, RMC-4948, RMC-0331, BI-1701963, BI-3406, SDR5, MRTX0902, BAY-293, and any combination thereof.

Embodiment 22: The method of any one of Embodiments 1-21, wherein the RAS(ON) GTP hydrolysis-promoting compound and the RAS(OFF) inhibitor, SHP2 inhibitor, RTK inhibitor or SOS1 inhibitor are administered on the same day.

Embodiment 23: The method of any one of Embodiments 1-21, wherein the RAS(ON) GTP hydrolysis-promoting compound and the RAS(OFF) inhibitor, SHP2 inhibitor, RTK inhibitor or SOS1 inhibitor are administered concurrently or sequentially.

Embodiment 24: The method of any one of Embodiments 1-21, wherein the RAS(ON) GTP hydrolysis-promoting compound and the RAS(OFF) inhibitor, SHP2 inhibitor, RTK inhibitor or SOS1 inhibitor are administered on different days.

Embodiment 25: The method of any one of Embodiments 1 to 22, wherein the method further comprises administering an additional anticancer therapy.

Embodiment 26: The method of Embodiment 25, wherein the additional anticancer therapy is an EGFR inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, an immune checkpoint inhibitor, a CDK4/6 inhibitor, a HER2 inhibitor, an RTK inhibitor, or a combination thereof.

Embodiment 27: The method of Embodiment 26, wherein the immune checkpoint inhibitor is a PD-L1 inhibitor or a PD-1 inhibitor.

Embodiment 28: A method of treating a RAS protein-related disorder in a subject in need thereof, the method comprising administering to the subject a therapeutically effective amount of a RAS(ON) GTP hydrolysis-promoting compound.

Embodiment 29: A method of treating a RASopathy in a subject in need thereof, the method comprising administering to the subject a therapeutically effective amount of a RAS(ON) GTP hydrolysis-promoting compound.

Embodiment 30: The method of embodiment 29, wherein the RASopathy is cardiofaciocutaneous syndrome, Costello syndrome, Legius syndrome, neurofibromatosis type 1, Noonan syndrome, or capillary malformation-arteriovenous malformation syndrome.

Embodiment 31: The method of any one of embodiments 28 to 30, wherein the method further comprises administering an additional RASopathy therapy.

Embodiment 32: The method of embodiment 31, wherein the additional RASopathy therapy is an EGFR inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, a CDK4/6 inhibitor, a HER2 inhibitor, an RTK inhibitor, or a combination thereof.

Embodiment 33: A method of inhibiting RAS activity in a cell, the method comprising contacting the cell with an effective amount of a RAS(ON) GTP hydrolysis-promoting compound.

Embodiment 34: A method of increasing sensitivity of a cell to a RAS(OFF) inhibitor, the method comprising contacting the cell with an effective amount of a RAS(ON) GTP hydrolysis-promoting compound, wherein the RAS(ON) GTP hydrolysis-promoting compound synergistically increases the sensitivity of the cell to the RAS(OFF) inhibitor.

Embodiment 35: A method of increasing the cross-linking rate of a KRAS^{G12C}(OFF) inhibitor to the cysteine residue at position 12 of KRAS^{G12C} in a cell comprising contacting the cell with an effective amount of a RAS(ON) GTP hydrolysis-promoting compound.

Embodiment 36: A pharmaceutical composition, comprising a therapeutically effective amount of a RAS(ON) GTP hydrolysis-promoting compound and a RAS(OFF) inhibitor.

Embodiment 37: A kit comprising, a) a RAS(ON) GTP hydrolysis-promoting compound and b) a RAS(OFF) inhibitor.

Embodiment 38: A kit comprising, a) a RAS(ON) GTP hydrolysis-promoting compound and b) a RTK inhibitor.

Embodiment 39: A kit comprising, a) a RAS(ON) GTP hydrolysis-promoting compound and b) a SHP2 inhibitor.

Embodiment 40: A kit comprising, a) a RAS(ON) GTP hydrolysis-promoting compound and b) a SOS1 inhibitor.

Embodiment 41: The kit of any one of Embodiments 37 to 40, further comprising an insert with instructions for administration of the pharmaceutical composition(s).

### Other Embodiments

While the disclosure has been described in connection with specific embodiments thereof, it will be understood that it is capable of further modifications and this application is intended to cover any variations, uses, or adaptations of the disclosure following, in general, the principles of the disclosure and including such departures from the disclosure that come within known or customary practice within the art to which the disclosure pertains and may be applied to the essential features hereinbefore set forth, and follows in the scope of the claims. Other embodiments are within the claims.

### EXAMPLES

The disclosure is further illustrated by the following examples and synthesis examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

### Example 1. Characterization of RAS(ON) GTP Hydrolysis-Promoting Compounds

The rate of RAS GTP hydrolysis was assayed in the presence of various KRAS mutations. To assess GTP hydrolysis activity, recombinant KRAS proteins (residue 1-169 of KRAS4B) were expressed in E. coli and purified using a TEV protease-cleavable His6-tag and Ni²⁺ affinity chromatography. The His6 tag was removed by treatment with TEV protease, and the KRAS protein was isolated by passing through a second Ni²⁺ column followed by size exclusion chromatography. The purified KRAS proteins were loaded with GTP by incubation for 2 hours on ice with 2 mM GTP and 10 mM Ethylenediaminetetraacetic Acid, followed by addition of 10 mM MgCl₂ and incubation on ice for 1 additional hour. The excess GTP was removed by overnight dialysis against buffer (12.5 mM HEPES, 75 mM NaCl, pH 7.5) at 4° C. The GTP-loaded KRAS protein were flash frozen in liquid nitrogen, then stored at −80° C. until use.

GTP-loaded KRAS proteins (1 μM) were combined with 25 μM of recombinant human cyclophilin A and 10 μM of compound in reaction buffer (12.5 mM HEPES, 75 mM NaCl, 1 mM MgCl₂, 1 mM DTT, 1% DMSO, pH 7.5) pre-warmed to 37° C. At fixed time points, aliquots were removed and quenched by heating to 80° C. to denature the proteins, then centrifuged to pellet the protein precipitate. The supernatant was assessed for GTP levels using Promega GTPase-Glo™ according to manufacturer instructions. The levels of GTP as a function of incubation time were fit to a single phase exponential decay to determine the rate constant for hydrolysis activity.

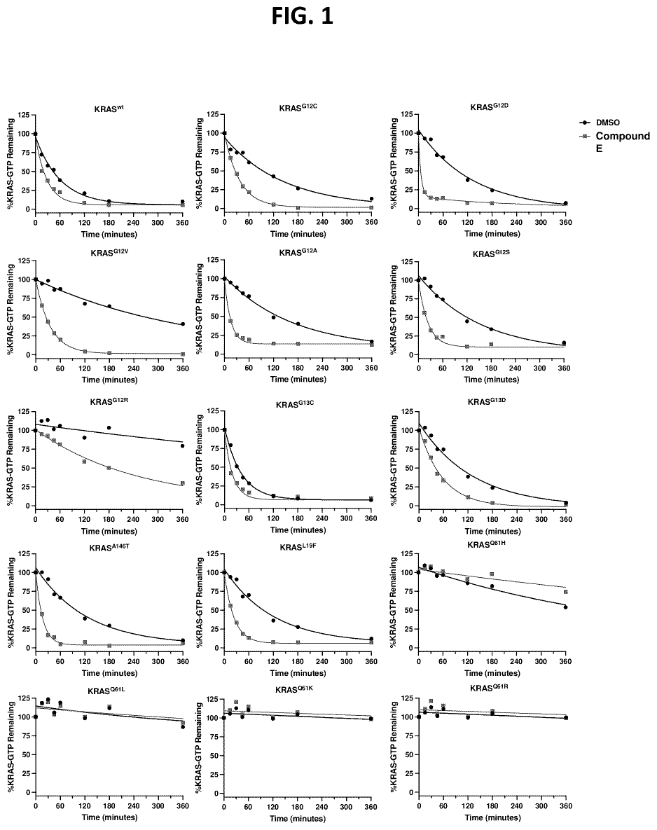

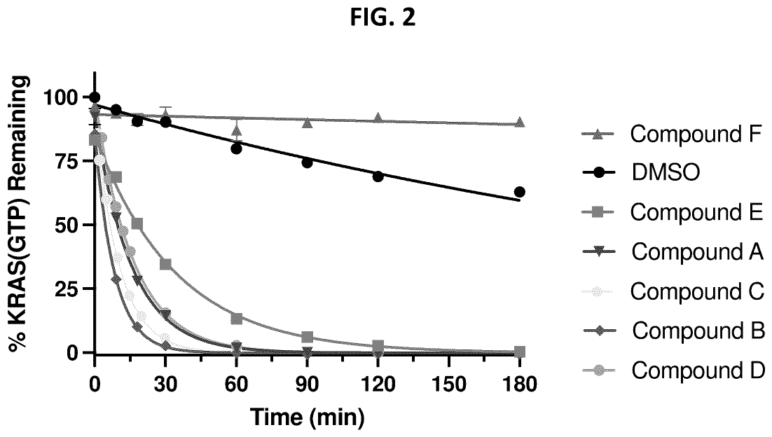

All RAS mutants show increased hydrolysis in the presence of a RAS(ON) GTP-hydrolysis promoting compound (Compound E), except those with mutations of the Q61 residue required for catalytical hydrolysis activity (FIG. 1). Next, RAS GTP hydrolysis activation was characterized using varying compounds. Compound A, Compound B and Compound C each represent a strong hydrolyzer, Compound D is a moderate hydrolyzer. Compound F represents the class that does not activate GTP hydrolysis by RAS, while the others show varying degrees of RAS GTP hydrolysis activation (FIG. 2).

The structure of Compound F is as follows:

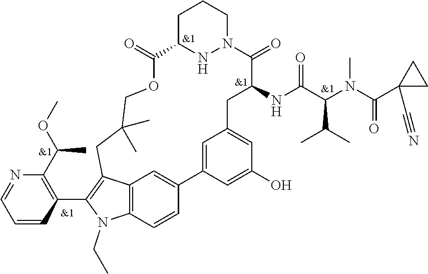

### Example 2. Synergy with the Combination of a RAS(ON) GTP Hydrolysis-Promoting Compound and a RAS Nucleotide Exchange Inhibitor

Cell lines were seeded in complete growth media (RPMI-1640 or DMEM with 10% Fetal Bovine Serum and 1% Penicillin/Streptomycin) and allowed to adhere overnight in a 37° C. and 5% CO2 humidified incubator. For phospho-(Thr202/Tyr204; Thr185/Tyr187)-ERK1/2 experiments, the following day compounds were added at the indicated concentrations and incubated for 4 hours. The cells were lysed and the amount of phospho-(Thr202/Tyr204; Thr185/Tyr187)-ERK1/2 relative to total ERK1/2 levels were assessed by Meso Scale Diagnostics Kit K15107D according to manufacturer instructions. For cell viability measurements, the following day compounds were added at the indicate concentrations and incubated for a further 5 days. The number of viable cells in each well were assessed with Promega CellTiter-Glo® reagent according to manufacturer instructions. The cell viability was normalized to DMSO controls. The levels of phospho-(Thr202/Tyr204; Thr185/Tyr187)-ERK1/2 or cell viability as a function of compound concentrations were plotted using GraphPad Prism. The EC50 values from a 4-parameter sigmoidal concentration-response value were used to calculate the fold-increase in potency as the ratio of the EC50 for the RAS(ON) compound in the absence of RMC-4550 to the EC50 for the RAS(ON) compound in the presence of RMC-4550.

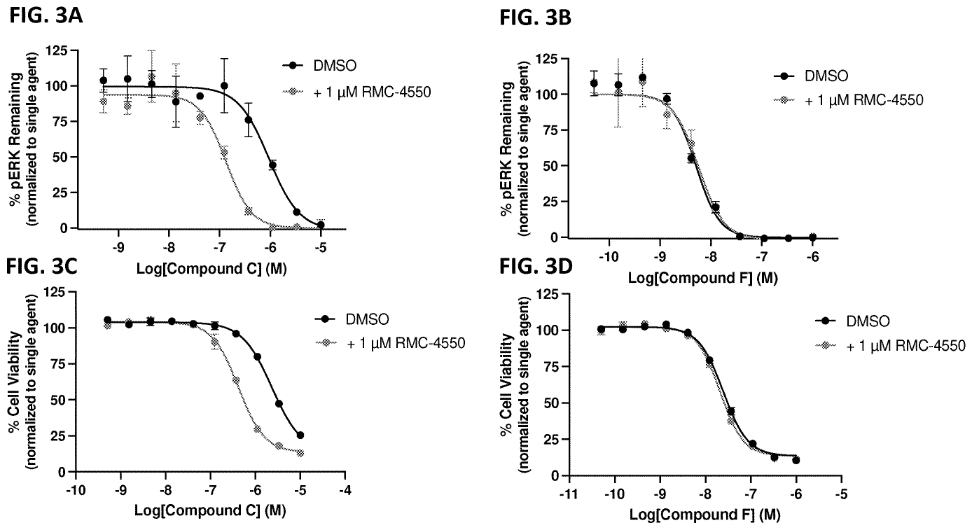

The KRASG12D-mutant cell line AsPC-1 was grown in RPMI-1640 supplemented with 10% fetal bovine serum and 1% penicillin/streptomycin. 20,000 cells were seeded in 0.10 mL in the wells of a tissue culture-treated 96-well plate. After overnight incubation, compounds were added at the indicated concentration and incubated for a further 4 hours. The amount of phospho-(Thr202/Tyr204; Thr185/Tyr187)-ERK1/2 relative to total ERK1/2 levels were assessed by Meso Scale Diagnostics Kit K15107D according to manufacturer instructions. The relative phospo-ERK levels was plotted as a function of the concentration of the strong hydrolyzer compound C (FIG. 3A) or the non-hydrolyzer compound F (FIG. 3B) in combination with DMSO or 1 μM RMC-4550. The KRASG12D-mutant cell line AsPC-1 was grown in RPMI-1640 supplemented with 10% fetal bovine serum and 1% penicillin/streptomycin. 4,000 cells were seeded in 0.10 mL in the wells of a tissue culture-treated 96-well plate. Cell viability was assessed with Promega CellTiter Glo reagent, and the relative cell viability was plotted as a function of the concentration of the strong hydrolyzer compound C (FIG. 3C) or the non-hydrolyzer compound F (FIG. 3D) in combination with DMSO or 1 μM RMC-4550.

### Example 3. Synergy with the Combination of a RAS(ON) GTP Hydrolysis-Promoting Compound and a RAS(OFF) Inhibitor

### HSA Synergy Model:

Cell lines were seeded in complete growth media (RPMI-1640 or DMEM with 10% Fetal Bovine Serum and 1% Penicillin/Streptomycin) and allowed to adhere overnight in a 37° C. and 5% CO₂ humidified incubator. The following day compounds were added at the indicate concentrations and incubated for a further 5 days. The number of viable cells in each well were assessed with Promega CellTiter-Glo® reagent according to manufacturer instructions. The cell viability was normalized to DMSO controls. The cell viability as a function of compound concentrations were plotted using GraphPad Prism analyzed using the Combenefit software package (Di Veroli G Y, Fornari C, Wang D, Mollard S, Bramhall J L, Richards F M, Jodrell D I. Combenefit: an interactive platform for the analysis and visualization of drug combinations. Bioinformatics. 2016 Sep. 15; 32(18):2866-8. doi: 10.1093/bioinformatics/btw230) to assess the synergy according to the highest single agent (HSA) synergy model (Borisy A A, Elliott P J, Hurst N W, Lee M S, Lehar J, Price E R, Serbedzija G, Zimmermann G R, Foley M A, Stockwell B R, Keith C T. Systematic discovery of multicomponent therapeutics. Proc Natl Acad Sci USA. 2003 Jun. 24; 100(13):7977-82. doi: 10.1073/pnas.1337088100)).

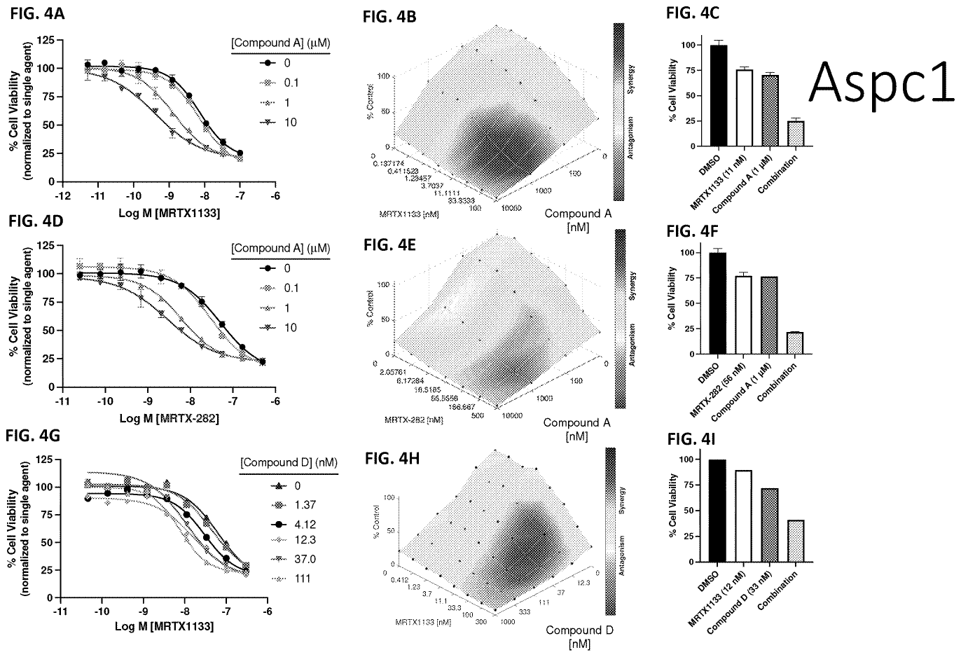

The KRASG12D-mutant cell line AsPC-1 was grown in RPMI-1640 supplemented with 10% fetal bovine serum and 1% penicillin/streptomycin. 2500 cells were seeded in 0.15 mL in the wells of a tissue culture-treated 96-well plate. After overnight incubation, compounds were added at the indicated concentration and incubated for a further 120 hours. Cell viability was assessed with Promega CellTiter Glo reagent, and the relative cell viability was plotted as a function of the concentration of (FIG. 4A and FIG. 4G) MRTX1133 or (FIG. 4D) MRTX-282 in combination with the indicated concentration to Compound A (FIG. 4A and FIG. 4D) or Compound D (FIG. 4G), both strong hydrolyzers. The synergy between (FIG. 4B and FIG. 4H) MRTX1133 or (FIG. 4E) MRTX-282 and Compound A (FIGS. 4B and 4E) or Compound D (FIG. 4H) was assessed by the HAS synergy model. Representative points of synergistic drug combination between (FIG. 4C and FIG. 4I) MRTX1133 or (FIG. 4F) MRTX-282 and Compound A (FIG. 4C and FIG. 4F) or Compound D (FIG. 4I) are highlighted.

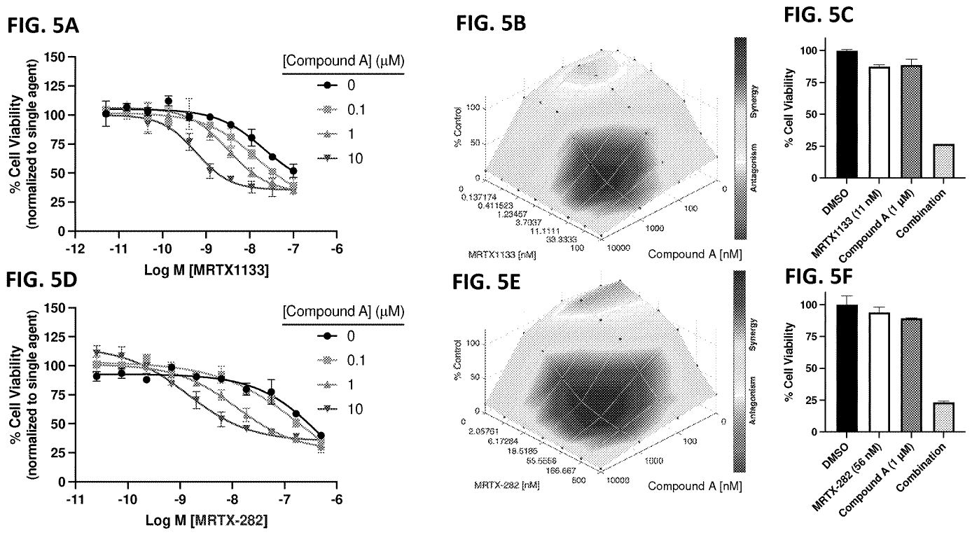

The KRASG12D-mutant cell line HPAC was grown in RPMI-16 40 supplemented with 10% fetal bovine serum and 1% penicillin/streptomycin. 2500 cells were seeded in 0.15 mL in the wells of a tissue culture-treated 96-well plate. After overnight incubation, compounds were added at the indicated concentration and incubated for a further 120 hours. Cell viability was assessed with Promega CellTiter Glo reagent, and the relative cell viability was plotted as a function of the concentration of (FIG. 5A) MRTX1133 or (FIG. 5D) MRTX-282 in combination with the indicated concentration to Compound A. The synergy between (FIG. 5B) MRTX1133 or (FIG. 5E) MRTX-282 and Compound A was assessed by the HAS synergy model. Representative points of synergistic drug combination between (FIG. 5C) MRTX1133 or (FIG. 5F) MRTX-282 and Compound A are highlighted.

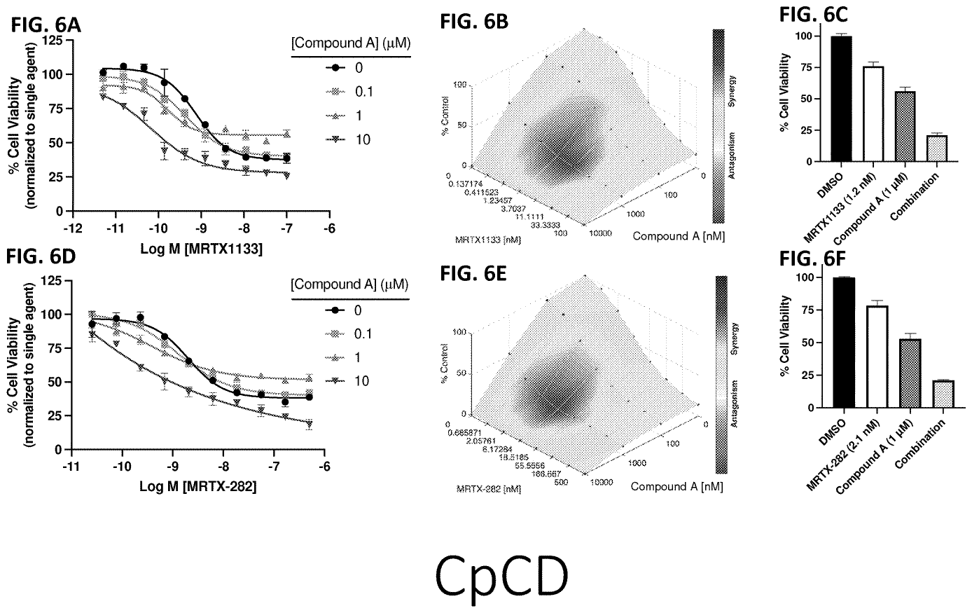

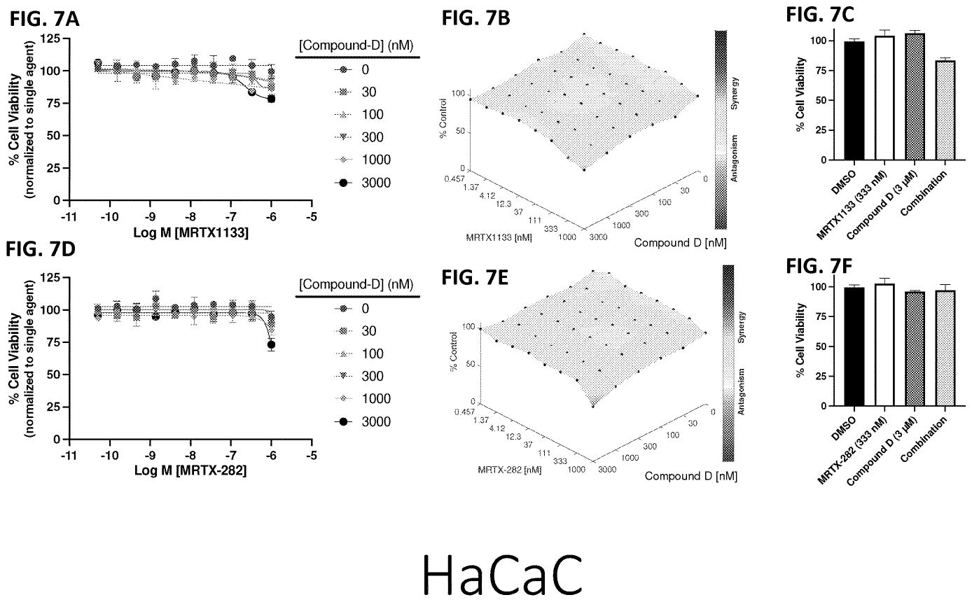

The KRASG12D-mutant cell line Gp2D was grown in RPMI-16 40 supplemented with 10% fetal bovine serum and 1% penicillin/streptomycin. 2500 cells were seeded in 0.15 mL in the wells of a tissue culture-treated 96-well plate. After overnight incubation, compounds were added at the indicated concentration and incubated for a further 120 hours. Cell viability was assessed with Promega CellTiter Glo reagent, and the relative cell viability was plotted as a function of the concentration of (FIG. 6A) MRTX1133 or (FIG. 6D) MRTX-282 in combination with the indicated concentration to Compound A. The synergy between (FIG. 6B) MRTX1133 or (FIG. 6E) MRTX-282 and Compound A was assessed by the HAS synergy model. Representative points of synergistic drug combination between (FIG. 6C) MRTX1133 or (FIG. 6F) MRTX-282 and Compound A are highlighted. As a control, the same experiments were performed in HaCat cells (RAS^{W}) using Compound D, MRTX1133, and MRTX-282. As expected, no synergy was observed (FIG. 7A-F). In the HaCaT cell line, a model of normal human skin keratinocytes, there is no increase in anti-proliferative effect when a RAS(ON) hydrolysis-promoting compound is combined with a RAS(OFF) inhibitor. Conversely, synergistic anti-proliferative effects of the combination of a RAS(ON) hydrolysis-promoting compound and a RAS(OFF) inhibitor are observed in cancer cell lines, as described above. The increased anti-proliferative effect of the combination therapy in cancer-derived cells, and not analogous normal cells, suggests an increased therapeutic effect without commitment toxicity on normal tissues.

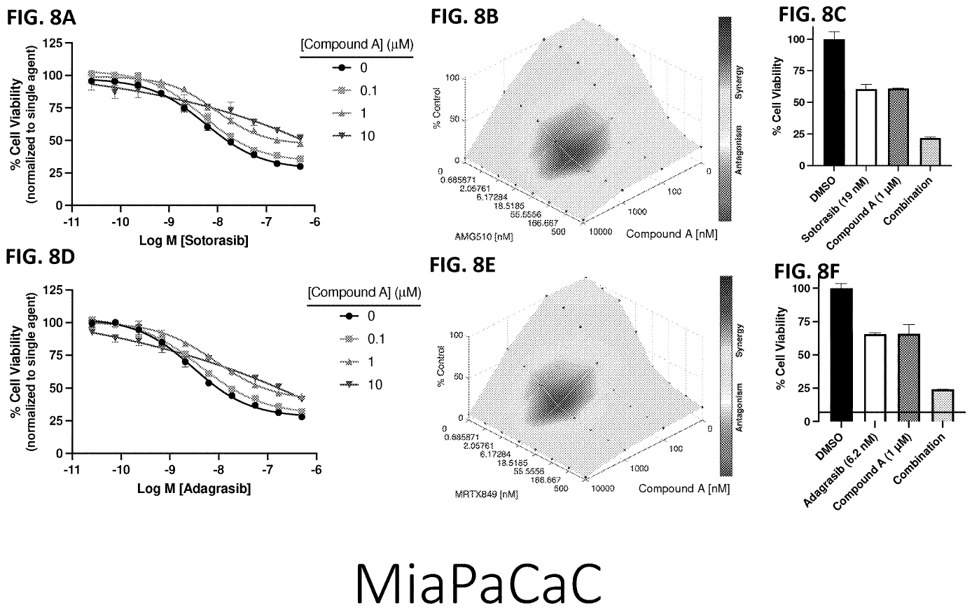

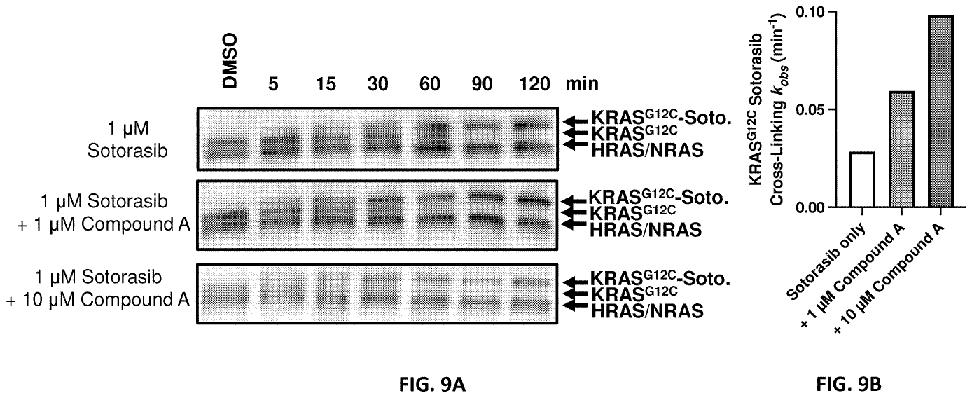

The KRASG12C-mutant cell line MiaPaCa2 was grown in DMEM supplemented with 10% fetal bovine serum and 1% penicillin/streptomycin. 2500 cells were seeded in 0.15 mL in the wells of a tissue culture-treated 96-well plate. After overnight incubation, compounds were added at the indicated concentration and incubated for a further 120 hours. Cell viability was assessed with Promega CellTiter Glo reagent, and the relative cell viability was plotted as a function of the concentration of (FIG. 8A) AMG510 or (FIG. 8D) MRTX849 in combination with the indicated concentration to Compound A. The synergy between (FIG. 8B) AMG510 or (FIG. 8E) MRTX849 and Compound A was assessed by the HAS synergy model. Representative points of synergistic drug combination between (FIG. 8C) AMG510 or (FIG. 8F) MRTX849 and Compound A are highlighted. In addition, the cross-linking efficiency was examined for 1 μM sotorasib alone, in combination with 1 μM Compound A or in combination with 10 μM Compound A (FIG. 9A-B). As can be seen, the cross-linking efficiency of sotorasib increases in a concentration dependent manner in the presence of Compound A. The KRAS^{G12C} inhibitor sotorasib covalently targets the GDP-bound OFF-state, resulting in a protein-drug adduct that can be observed by reduced mobility by SDS denaturing polyacrylamide gel electrophoresis. When MiaPaca-2 cells were treated with sotorasib, the amount of covalently-modified KRAS^{G12C} (KRAS^{G}12C-Soto.) increased with increasing incubation time. When the same cells were treated with sotorasib in combination with 1 or 10 μM of Compound A, the amount of KRAS=-Soto. is increased at each time point. The density of the KRAS^{G12}C and KRAS^{G12C}-Soto. gel bands can be quantified, plotted as a function of time, and fit to a single-phase exponential model to get the rate constant for KRAS^{G12C} target engagement. The combination of sotorasib and compound A results in more rapid covalent engagement of KRAS^{G12C} as compared with sotorasib alone.

A cellular assay was used to measure complexes between KRAS proteins and the RAS-binding domain (RBD) of the RAF1 kinase by nanoluciferase bioluminescence energy transfer (nanoBRET™) U2OS cells were seeded at confluence in the 3 mL of DMEM supplemented with 10% fetal bovine serum and 1% penicillin/streptomycin (approximately 1 million cells per well). The following day, each well was transfected with 50 ng of a plasmid encoding N-terminal nanoluciferase-tagged KRAS4B and 2950 ng of a plasmid encoding a C-terminal HaloTag® RAF1 RBD (residues 51-149) using FuGENE HD reagent according to manufacturer instructions. The following day, each transfected cell pool was transferred to 36 wells of a white 96-well plate in media consistiting of OptiMem phenol-red free supplemented with 4% fetal bovine serum, 1% penicillin/streptomycin, and 100 nM of HaloTag® NanoBRET™ 618 Ligand. The following day, Vivazine™ nanoluciferase substrate and compounds were added at the indicated concentration. After 4 hours of incubation, the emitted light at 460 and 618 nm was measured using a PerkinElmer Envision plate reader. The luminescence intensity at 618 nm relative to 460 nm is proportional to the number of complexes between the KRAS protein and the RBD of RAF1, a surrogate measure of the levels of active KRAS.

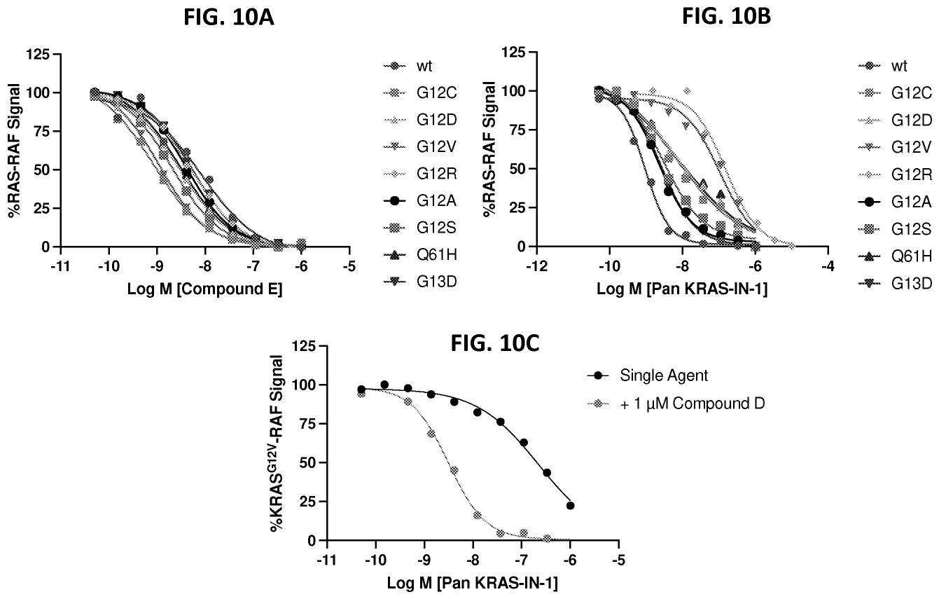

Compound E exhibited roughly similar potency for inhibition of RAS-RAF complexes across multiple KRAS variants, with modestly lower potency vs KRAS^{WT} (FIG. 10A). In contrast, an exemplar pan-KRAS(OFF) inhibitor, pan KRAS-IN-1 (CAS No.: 2791263-84-6):

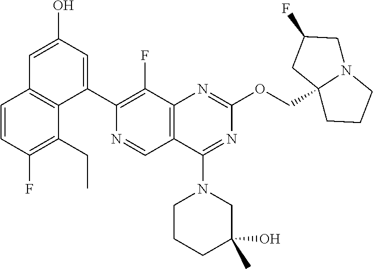 [SMILES: CCc1c(F)ccc2cc(O)cc(-c3ncc4c(N5CCC[C@@](C)(O)C5)nc(OC[C@@]56CCCN5C[C@H](F)C6)nc4c3F)c12]

exhibited greatest potency vs RAS^{WT} (FIG. 10B). The potency for the various KRAS mutants roughly correlated with the anticipated rate of hydrolysis and was lowest at KRAS^{G12V} and KRAS^{G12R}. Coincubation with a RVMD super hydrolyser increased the potency of the pan-KRAS(OFF) inhibitor for inhibiting KRAS^{G12V} (FIG. 10C).

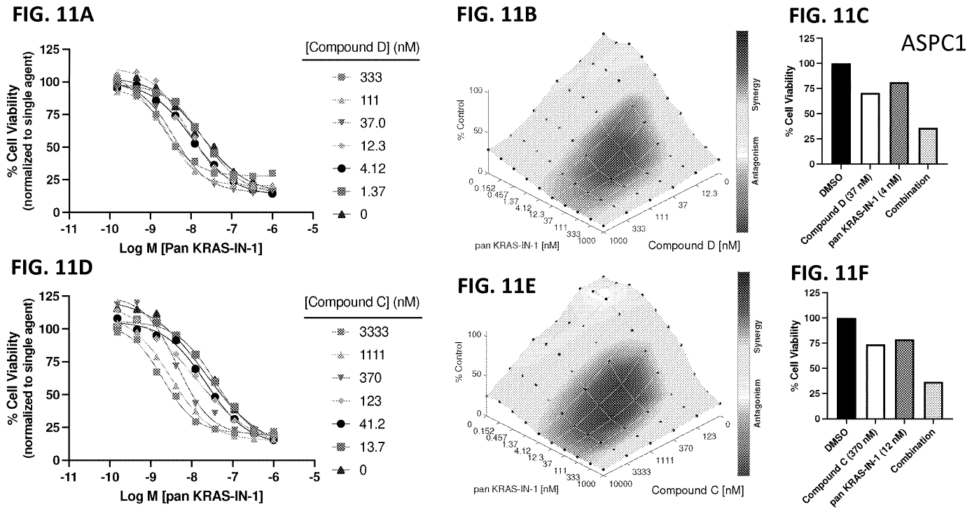

The KRASG12D-mutant cell line AsPC-1 was grown in RPMI-1640 supplemented with 10% fetal bovine serum and 1% penicillin/streptomycin. 2000 cells were seeded in 0.15 mL in the wells of a tissue culture-treated 96-well plate. After overnight incubation, compounds were added at the indicated concentration and incubated for a further 120 hours. Cell viability was assessed with Promega CellTiter Glo reagent, and the relative cell viability was plotted as a function of the concentration of pan KRAS-IN-1 in combination with the indicated concentration to Compound D (FIG. 11A) or Compound C (FIG. 11D), both strong hydrolyzers. The synergy between pan KRAS-IN-1 and Compound D (FIG. 11B) or Compound C (FIG. 11E) was assessed by the HAS synergy model. Representative points of synergistic drug combination between pan KRAS-IN-1 and Compound D (FIG. 11C) or Compound C (FIG. 11F).

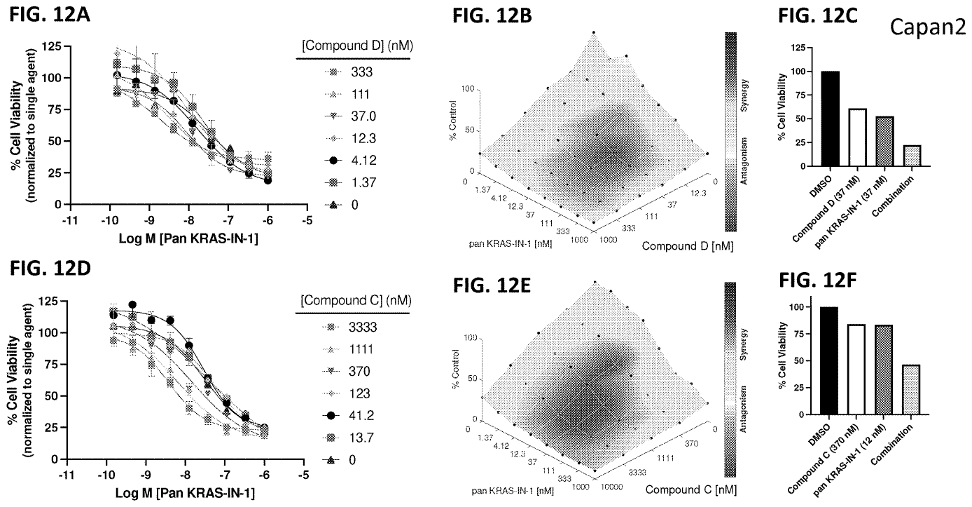

The KRASG12V-mutant cell line Capan-2 was grown in RPMI-1640 supplemented with 10% fetal bovine serum and 1% penicillin/streptomycin. 2000 cells were seeded in 0.15 mL in the wells of a tissue culture-treated 96-well plate. After overnight incubation, compounds were added at the indicated concentration and incubated for a further 120 hours. Cell viability was assessed with Promega CellTiter Glo reagent, and the relative cell viability was plotted as a function of the concentration of pan KRAS-IN-1 in combination with the indicated concentration to Compound D (FIG. 12A) or Compound C (FIG. 12D), both strong hydrolyzers. The synergy between pan KRAS-IN-1 and Compound D (FIG. 12B) or Compound C (FIG. 12E) was assessed by the HAS synergy model. Representative points of synergistic drug combination between pan KRAS-IN-1 and Compound D (FIG. 12C) or Compound C (FIG. 12F).

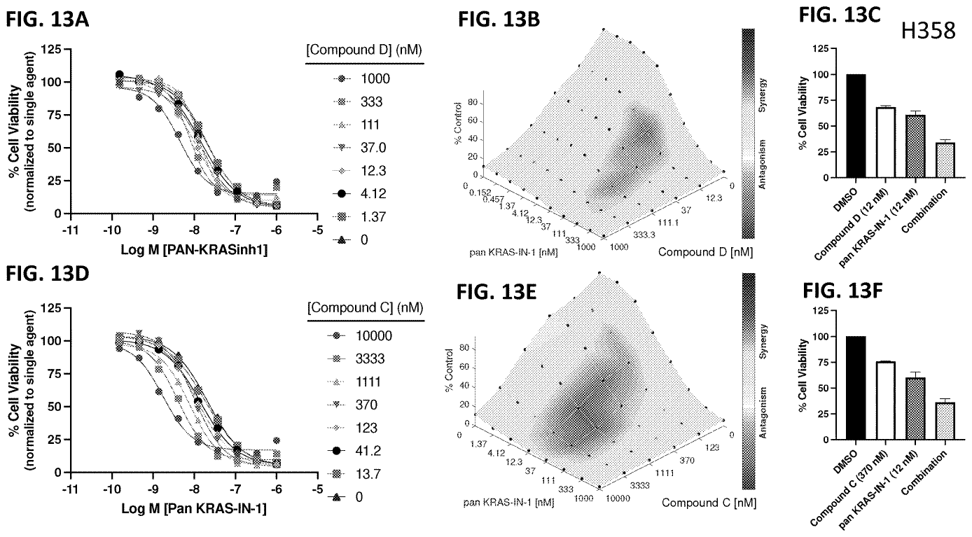

The KRASG12C-mutant cell line H358 was grown in RPMI-1640 supplemented with 10% fetal bovine serum and 1% penicillin/streptomycin. 2000 cells were seeded in 0.15 mL in the wells of a tissue culture-treated 96-well plate. After overnight incubation, compounds were added at the indicated concentration and incubated for a further 120 hours. Cell viability was assessed with Promega CellTiter Glo reagent, and the relative cell viability was plotted as a function of the concentration of pan KRAS-IN-1 in combination with the indicated concentration to Compound D (FIG. 13A) or Compound C (FIG. 13D), both strong hydrolyzers. The synergy between pan KRAS-IN-1 and Compound D (FIG. 13B) or Compound C (FIG. 13E) was assessed by the HAS synergy model. Representative points of synergistic drug combination between pan KRAS-IN-1 and Compound D (FIG. 13C) or Compound C (FIG. 13F).

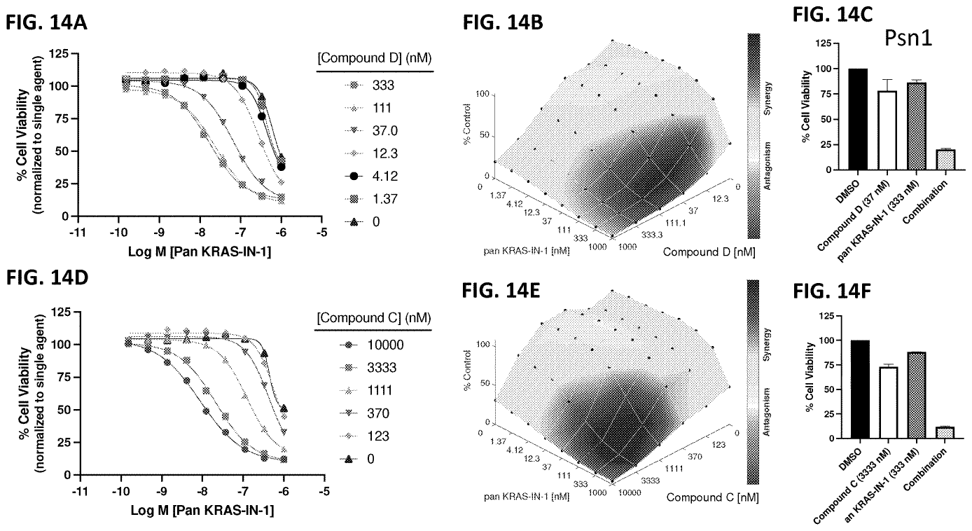

The KRASG12R-mutant cell line PSN1 was grown in RPMI-1640 supplemented with 10% fetal bovine serum and 1% penicillin/streptomycin. 2000 cells were seeded in 0.15 mL in the wells of a tissue culture-treated 96-well plate. After overnight incubation, compounds were added at the indicated concentration and incubated for a further 120 hours. Cell viability was assessed with Promega CellTiter Glo reagent, and the relative cell viability was plotted as a function of the concentration of pan KRAS-IN-1 in combination with the indicated concentration to Compound D (FIG. 14A) or Compound C (FIG. 14D), both strong hydrolyzers. The synergy between pan KRAS-IN-1 and Compound D (FIG. 14B) or Compound C (FIG. 14E) was assessed by the HAS synergy model. Representative points of synergistic drug combination between pan KRAS-IN-1 and Compound D (FIG. 14C) or Compound C (FIG. 14F).

1. A method of treating cancer in a subject in need thereof, the method comprising administering to the subject a therapeutically effective amount of a RAS(ON) GTP hydrolysis-promoting compound and a RAS(OFF) inhibitor, wherein the cancer does not comprise a RAS mutation at position 61.

2-5. (canceled)

6. The method of claim 1, wherein the method further comprises administering a RAS(OFF) inhibitor.

7. (canceled)

8. The method of claim 1, wherein the cancer comprises a RAS mutation.

9. The method of claim 8, wherein the RAS mutation is at position 12 or 13.

10. The method of claim 1, wherein the cancer is pancreatic cancer, colorectal cancer, non-small cell lung cancer, gastric cancer, esophageal cancer, ovarian cancer, or uterine cancer.

11. The method of claim 1, wherein the method further comprises administering an additional anticancer therapy.

12. The method of claim 11, wherein the additional anticancer therapy is an EGFR inhibitor, a SHP2 inhibitor, a SOS1 inhibitor, a Raf inhibitor, a MEK inhibitor, an ERK inhibitor, a PI3K inhibitor, a PTEN inhibitor, an AKT inhibitor, an mTORC1 inhibitor, a BRAF inhibitor, an immune checkpoint inhibitor, a CDK4/6 inhibitor, a HER2 inhibitor, an RTK inhibitor, or a combination thereof.

13-14. (canceled)

15. A method of increasing sensitivity of a cell to a RAS(OFF) inhibitor, the method comprising contacting the cell with an effective amount of a RAS(ON) GTP hydrolysis-promoting compound, wherein the RAS(ON) GTP hydrolysis-promoting compound synergistically increases the sensitivity of the cell to the RAS(OFF) inhibitor.

16. The method of claim 15, wherein the RAS(OFF) inhibitor is a KRAS(OFF) inhibitor.

17. The method of claim 16, wherein the KRAS(OFF) inhibitor is a KRAS^{G12C}(OFF) inhibitor.

18. The method of claim 16, wherein the KRAS(OFF) inhibitor is a KRAS^{G12D}(OFF) inhibitor.

19. The method of claim 16, wherein the KRAS(OFF) inhibitor is a KRAS^{G12V}(OFF) inhibitor.

20. The method of claim 16, wherein the KRAS(OFF) inhibitor is a pan-RAS(OFF) inhibitor.

21-23. (canceled)

24. The method of claim 1, wherein the RAS(ON) GTP-hydrolysis promoting compound is a compound of Table 1 or a pharmaceutically acceptable salt thereof.

25-26. (canceled)

27. The method of claim 1, wherein the RAS(ON) GTP-hydrolysis promoting compound is a compound having the structure of Formula Ic, Id or Ie: 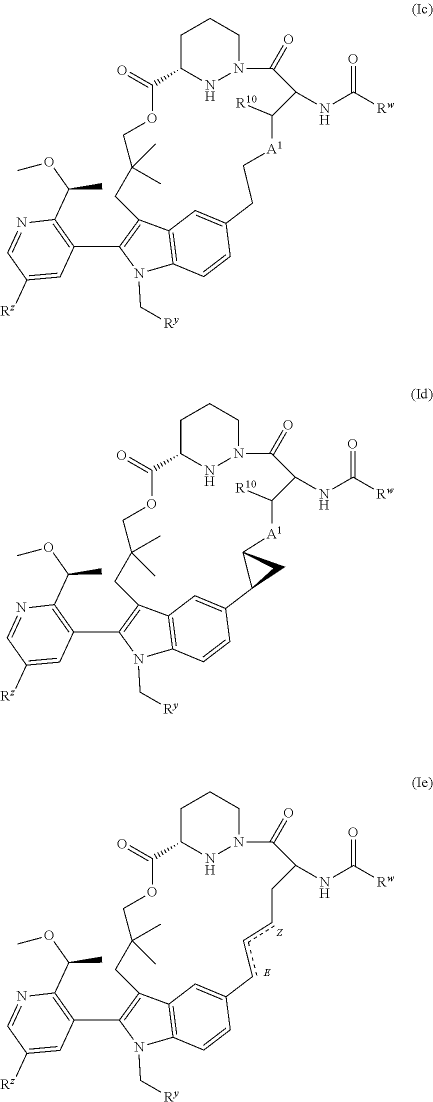
or a pharmaceutically acceptable salt thereof, wherein:
in Formula Ic:
R^{w} is methylcyclopropyl;
R^{y} is CH₃, CH₂F, CHF, or CHF₃;
R^{z} is hydrogen or N-methylpiperazinyl;
R¹⁰ is H; and
A¹ is —CH₂—, —O—, or —NCH₃;
in Formula Id:
R^{w} is methylcyclopropyl or dimethylcyclopropyl;
R^{y} is CH₃;
R^{z} is hydrogen or N-methylpiperazinyl;
R¹⁰ is H; and
A¹ is —CH₂, —O— or —NCH₃; or
in Formula Ie:
either the E or Z double bond is present;
R^{w} is methylcyclopropyl or dimethylcyclopropyl;
R^{y} is CH₃;
R^{z} is hydrogen or N-methylpiperazinyl;
R¹⁰ is H; and
A¹ is —CH₂—.

28. A pharmaceutical composition comprising (i) a means for promoting RAS(ON) GTP hydrolysis, and (ii) a pharmaceutically acceptable excipient.

29. A pharmaceutical composition comprising (i) a means for altering the position of glutamine 61 of RAS(ON) towards the gamma phosphate of GTP bound to the RAS(ON) protein, thereby increasing the GTP hydrolysis rate relative to the hydrolysis rate of RAS(ON) in the absence of said means, and (ii) a pharmaceutically acceptable excipient.
